(19) United States
(12) Patent Application Publication
(10) Pub. No.: US 2023/0124464 A1
(43) Pub. Date: Apr. 20, 2023

(54) CHIMERIC RECEPTORS TO FLT3 AND METHODS OF USE THEREOF

(71) Applicants: Amgen, Inc., Thousand Oaks, CA (US); Kite Pharma, Inc., Santa Monica, CA (US)

(72) Inventors: Alice BAKKER, Cupertino, CA (US); Lawren WU, Foster City, CA (US); Tara ARVEDSON, Moss Beach, CA (US); Jed J. WILTZIUS, Santa Monica, CA (US); Ruben Alvarez RODRIGUEZ, Los Angeles, CA (US)

(21) Appl. No.: 17/751,586

(22) Filed: May 23, 2022

Related U.S. Application Data
(62) Division of application No. 16/090,562, filed on Oct. 1, 2018, now abandoned.
(60) Provisional application No. 62/317,219, filed on Apr. 1, 2016.

(51) Int. Cl. A61K 35/17; C07K 16/28; C12N 9/12; A61P 37/06; A61P 35/02; C07K 14/725; C07K 14/705; C12N 15/62; C12N 15/85

(52) U.S. Cl. CPC A61K 35/17; C07K 16/2863; C12Y 207/10001; C12N 9/12; A61P 37/06; A61P 35/02; C07K 14/7051; C07K 14/70521; C12N 15/62; C12N 15/85; C07K 2317/622; C07K 2319/33; C07K 2319/03; A61K 2039/505

(57) ABSTRACT

Antigen binding molecules, chimeric receptors, and engineered immune cells to FLT3 are disclosed in accordance with the invention. The invention further relates to vectors, compositions, and methods of treatment and/or detection using the FLT3 antigen binding molecules and engineered immune cells.

### BACKGROUND OF THE INVENTION

Acute Myeloid Leukemia (AML) is a heterogenous hematological malignancy that is the most common type of acute leukemia diagnosed in adults. AML accounts for roughly a third of all leukemias with an estimated 14,500 new cases reported in 2013 in the United States alone and poor overall survival rates. There has been little improvement in the standard of care for AML patients over the past thirty years. However, recent advances in molecular and cell biology have revolutionized our understanding of human hematopoiesis, both in normal and diseased states.

Several key players involved in disease pathogenesis have been identified and can be interrogated as actionable targets. One such activating “driver” gene that is most commonly mutated in approximately 30% of AML is FLT3.

Fms-like tyrosine kinase 3 (FLT3) also known as fetal liver kinase 2 (FLK-2), human stem cell kinase 1 (SCK-1) or Cluster of Differentiation antigen (CD135) is a hematopoietic receptor tyrosine kinase that was cloned by two independent groups in the 1990s. The FLT3 gene, located on chromosome 13q12 in humans encodes a Class III receptor tyrosine kinase protein that shares homology with other Class III family members including stem cell factor receptor (c-KIT), macrophage colony-stimulating factor receptor (FMS) and platelet-derived growth factor receptor (PDGFR).

Upon binding with the FLT3 ligand, FLT3 receptor undergoes homodimerization thereby enabling autophosphorylation of specific tyrosine residues in the juxtamembrane domain and downstream activation via PI3K/Akt, MAPK and STATS pathways. FLT3 thus plays a crucial role in controlling proliferation, survival and differentiation of normal hematopoietic cells.

Human FLT3 is expressed in CD34+CD38− hematopoietic stem cells (HSC) as well as in a subset of dendritic precursor cells. FLT3 expression can also be detected in multipotent progenitor cells like the CD34⁺CD38⁺CD45RA⁻CD123^{low} Common Myeloid Progenitor (CMP), CD34⁺CD38⁺CD45RA⁺CD123^{low} Granulocyte Monocyte Progenitors (GMP), and CD34^{|}CD38^{|}CD10^{|}CD19⁻ Common Lymphoid Progenitor cells (CLP). Interestingly, FLT3 expression is almost absent in the CD34⁺CD38⁻CD45RA⁻CD123⁻ Megakaryocyte Erythrocyte Progenitor cells (MEP). FLT3 expression is thus confined mainly to the early myeloid and lymphoid progenitor cells with some expression in the more mature monocytic lineage cells. This limited expression pattern of FLT3 is in striking contrast to that of FLT3 ligand, which is expressed in most hematopoietic tissues and the prostate, kidney, lung, colon and heart. These varied expression patterns such that FLT3 expression is the rate limiting step in determining tissue specificity of FLT3 signaling pathways.

The most common FLT3 mutation in AML is the FLT3 internal tandem duplication (FLT3-ITD) that is found in 20 to 38% of patients with cytogenetically normal AML. FLT3-ITDs are formed when a portion of the juxtamembrane domain coding sequence gets duplicated and inserted in a head to tall orientation. FLT3 mutations have not been identified in patients with chronic lymphoid leukemia (CLL), non—Hodgkin's lymphoma and multiple myeloma suggesting strong disease specificity for AML. Mutant FLT3 activation is generally observed across all FAB subtypes, however, it is significantly increased in AML patients with FAB M5 (monocytic leukemia), while FAB subtypes M2 and M6 (granulocytic or erythroid leukemia) are significantly less frequently associated with FLT3 activation, in line with normal expression patterns of FLT3. A small percentage of AML patients (5-7%) present with single amino acid mutations in the FLT3 tyrosine kinase domain (FLT3 TKD) , most commonly at D835 or in some cases at T842 or I836 while even fewer patients (˜1%) harbor mutations in the FLT3 juxtamembrane domain involving residues 579, 590, 591 and 594. Patients with FLT3-ITD mutant AML have an aggressive form of disease characterized by early relapse and poor survival, while overall survival and event-free survival are not significantly influenced by presence of FLT3-TKD mutations. Furthermore, AML patients with FLT3-ITD mutation with concurrent TET2 or DNMT3A mutations have an unfavorable overall risk profile compared to FLT3-ITD mutant AML patients with wild-type TET2 or DNMT3A underscoring the clinical and biological heterogeneity of AML.

Both FLT3-ITD and FLT3 TKD mutations induce ligand independent activation of FLT3 leading to downstream activation of the Ras/MAPK pathway and the PI3K/Akt pathways. However, the downstream signaling pathways associated with either mutation differ primarily in the preferential activation of STATS by FLT3-ITD, thereby leading to increased proliferation potential and aberrant regulation of DNA repair pathways.

Independent of FLT3 mutation status, FLT3 phosphorylation is evident in over two-thirds of AML patients and FLT3 is expressed in >80% AML blasts and in ˜90% of all AML patients making it an attractive therapeutic target associated with disease pathogenesis in a large sample size.

Several small molecule inhibitors have emerged as attractive therapeutic options for AML patients with FLT3 mutations. The first generation of FLT3 tyrosine kinase inhibitors (TKI) was characterized by lack of selectivity, potency and unfavorable pharmacokinetic properties. Newer and more selective agents have been developed to combat this issue; however, their efficacy has been limited by emergence of secondary resistance.

Several early FLT3 TKIs included midostaurin (PKC412), lestaurtinib (CEP-701), sunitinib (SUI1248) and sorafinib (BAY 43-9006) amongst others. Response rates in Phase I and Phase II with these multikinase targeting agents in patients with relapsed or refractory AML is limited, presumably due to their inability to achieve effective FLT3 inhibition without dose limiting toxicities. Quizartinib (AC220) has been developed as a second generation FLT3 TM with high selectivity for FLT3 wild type and FLT3-ITD and has demonstrated benefit especially in the peritransplant setting in a younger cohort of patients. However, secondary mutations in FLT3 identified in relapsed patients who received quizartinib accentuate the need to develop better therapeutic strategies for AML patients, while highlighting the validity of FLT3 as a therapeutic target.

Several targeted agents have been tested in AML patients with either de novo, relapsed/refractory or secondary disease. Epigenetic silencing of tumor suppressor genes plays an important role in AML disease pathogenesis, and DNA methyltransferase (DNMT) inhibitors like azacitadine and decitabine have achieved some clinical success. Further, the recent identification of mutations that affect histone posttranslational modifications (e.g. EZH2 and ASXL1 mutations) or DNA methylation (e.g. DNMT3A, TET2, IDH1/2) in a subset of AML patients has led to development of a variety of therapeutic options including EZH2, DOT1L, IDH1/2 inhibitors along with HDAC and proteasome inhibitors. However, preclinical studies of many of these compounds in AML cells suggest that these inhibitors may be altering the phenotype and gene expression characteristic of hematopoietic differentiation rather than causing direct cytotoxicity of AML blasts. There therefore remains a strong unmet medical need to identify novel targets/modalities to combat AML and cause targeted lysis of AML blast cells. Other therapeutic candidates for AML include Aurora kinase inhibitors including AMG 900 and inhibitors to polo-like kinases that play an important role in cell cycle progression.

The standard of care for AML patients has remained chemotherapy with stem cell transplantation when feasible. However the emergence of relapsed/refractory cases in a large majority of treated patients warrants additional therapeutic modalities. The identification and description of several leukemia specific antigens along with a clearer understanding of immune mediated graft-versus-leukemia effects have paved the way to development of immunomodulatory strategies for combating hematological malignancies, reviewed in several articles.

Engineered immune cells have been shown to possess desired qualities in therapeutic treatments, particularly in oncology. Two main types of engineered immune cells are those that contain chimeric antigen receptors (termed “CARS” or “CAR-Ts”) and T-cell receptors (“TCRs”). These engineered cells are engineered to endow them with antigen specificity while retaining or enhancing their ability to recognize and kill a target cell. Chimeric antigen receptors may comprise, for example, (i) an antigen-specific component (“antigen binding molecule”), (ii) one or more costimulatory domains, and (iii) one or more activating domains. Each domain may be heterogeneous, that is, comprised of sequences derived from different protein chains. Chimeric antigen receptor-expressing immune cells (such as T cells) may be used in various therapies, including cancer therapies. It will be appreciated that costimulating polypeptides as defined herein may be used to enhance the activation of CAR-expressing cells against target antigens, and therefore increase the potency of adoptive immunotherapy.

T cells can be engineered to possess specificity to one or more desired targets. For example, T cells can be transduced with DNA or other genetic material encoding an antigen binding molecule, such as one or more single chain variable fragment (“scFv”) of an antibody, in conjunction with one or more signaling molecules, and/or one or more activating domains, such as CD3 zeta.

In addition to the CAR-T cells' ability to recognize and destroy the targeted cells, successful T cell therapy benefits from the CAR-T cells' ability to persist and maintain the ability to proliferate in response to antigen.

T cell receptors (TCRs) are molecules found on the surface of T cells that are responsible for recognizing antigen fragments as peptides bound to major histocompatibility complex (MHC) molecules. The TCR is comprised of two different protein chains—in approximately 95% of human TCRs, the TCR consists of an alpha (α) and beta (β) chain. In approximately 5% of human T cells the TCR consists of gamma and delta (γ/δ) chains. Each chain is composed of two extracellular domains: a variable (V) region and a constant (C) region, both of the immunoglobulin superfamily. As in other immunoglobulins, the variable domains of the TCR α-chain and β-chain (or gamma and delta (γ/δ) chains) each have three hypervariable or complementarity determining regions (CDRs). When the TCR engages with antigenic peptide and MHC (peptide/MHC), the T cell becomes activated, enabling it to attack and destroy the target cell.

However, current therapies have shown varying levels of effectiveness with undesired side effects. Therefore, a need exists to identify novel and improved therapies for treating FLT3 related diseases and disorders.

### SUMMARY OF THE INVENTION

The invention relates to engineered immune cells (such as CARs or TCRs), antigen binding molecules (including but not limited to, antibodies, scFvs, heavy and/or light chains, and CDRs of these antigen binding molecules) with specificity to FLT3.

The invention further relates to a novel CD28 sequence useful as costimulatory domains in these cells.

Chimeric antigen receptors of the invention typically comprise: (i) a FLT3 specific antigen binding molecule, (ii) one or more costimulatory domain, and (iii) one or more activating domain. It will be appreciated that each domain may be heterogeneous, thus comprised of sequences derived from different protein chains.

In some embodiments, the invention relates to a chimeric antigen receptor comprising an antigen binding molecule that specifically binds to FLT3, wherein the antigen binding molecule comprises at least one of: (a) a variable heavy chain CDR1 comprising an amino acid sequence differing from that of SEQ ID NO: 17 by not more than 3, 2, 1, or 0 amino acid residues; (b) a variable heavy chain CDR2 comprising an amino acid sequence differing from that of SEQ ID NO:18 or SEQ ID NO:26 by not more than 3, 2, 1, or 0 amino acid residues; (c) a variable heavy chain CDR3 comprising an amino acid sequence differing from that of SEQ ID NOs SEQ ID NO: 19 or SEQ ID NO:27 by not more than 3, 2, 1, or 0 amino acid residues; (d) a variable light chain CDR1 comprising an amino acid sequence differing from that of SEQ ID NO:22 or SEQ ID NO:30 by not more than 3, 2, 1, or 0 amino acid residues; (e) a variable light chain CDR2 comprising an amino acid sequence differing from that of SEQ ID NO:23 or 31 by not more than 3, 2, 1, or 0 amino acid residues; (f) a variable light chain CDR3 comprising an amino acid sequence differing from that of SEQ ID:24 or SEQ ID NO:32 by not more than 3, 2, 1, or 0 amino acid residues.

In other embodiments, the chimeric antigen receptor further comprises at least one costimulatory domain. In further embodiments, the chimeric antigen receptor further comprises at least one activating domain.

In certain embodiments the costimulatory domain is a signaling region of CD28, CD28T, OX-40, 4-1BB/CD137, CD2, CD7, CD27, CD30, CD40, Programmed Death-1 (PD-1), inducible T cell costimulator (ICOS), lymphocyte function-associated antigen-1 (LFA-1, CDl-la/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CDlld, ITGAE, CD103, ITGAL, CD11a, LFA-1, ITGAM, CD11b, ITGAX, CD11c, ITGBl, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

In some embodiments, the costimulatory domain is derived from 4-1BB. In other embodiments, the costimulatory domain is derived from OX40. See also Hombach et al., Oncoimmunology. 2012 Jul. 1; 1(4): 458-466. In still other embodiments, the costimulatory domain comprises ICOS as described in Guedan et al., Aug. 14, 2014; Blood: 124 (7) and Shen et al., Journal of Hematology & Oncology (2013) 6:33. In still other embodiments, the costimulatory domain comprises CD27 as described in Song et al., Oncoimmunology. 2012 Jul. 1; 1(4): 547-549.

In certain embodiments, the CD28 costimulatory domain comprises SEQ ID NO: 2, SEQ ID NO: 4, SEQ ID NO: 6, or SEQ ID NO: 8. In additional embodiments, the CD8 costimulatory domain comprises SEQ ID NO: 14. In further embodiments, the activating domain comprises CD3, CD3 zeta, or CD3 zeta having the sequence set forth in SEQ ID NO: 10.

In other embodiments, the invention relates to a chimeric antigen receptor wherein the costimulatory domain comprises SEQ ID NO: 2 and the activating domain comprises SEQ ID NO: 10.

The invention further relates to polynucleotides encoding the chimeric antigen receptors, and vectors comprising the polynucleotides. The vector can be, for example, a retroviral vector, a DNA vector, a plasmid, a RNA vector, an adenoviral vector, an adenovirus associated vector, a lentiviral vector, or any combination thereof The invention further relates to immune cells comprising the vectors. In some embodiments, the lentiviral vector is a pGAR vector.

Exemplary immune cells include, but are not limited to T cells, tumor infiltrating lymphocytes (TILs), NK cells, TCR-expressing cells, dendritic cells, or NK-T cells. The T cells can be autologous, allogeneic, or heterologous. In other embodiments, the invention relates to pharmaceutical compositions comprising the immune cells of described herein.

In certain embodiments, the invention relates to antigen binding molecules (and chimeric antigen receptors comprising these molecules) comprising at least one of:
(a) a VH region differing from the amino acid sequence of the VH region of 10E3 by no more than 10, 9, 8, 7, 6, 5, 4, 3, 2, 1, or 0 amino acid residues and a VL region differing from the amino acid sequence of the VL region of 10E3 by no more than 10, 9, 8, 7, 6, 5, 4, 3, 2, 1, or 0 amino acid residues;
(b) a VH region differing from the amino acid sequence of the VH region of 2E7 by no more than 10, 9, 8, 7, 6, 5, 4, 3, 2, 1, or 0 amino acid residues and a VL region differing from the amino acid sequence of the VL region of 2E7 by no more than 10, 9, 8, 7, 6, 5, 4, 3, 2, 1, or 0 amino acid residues;
(c) a VH region differing from the amino acid sequence of the VH region of 8B5 by no more than 10, 9, 8, 7, 6, 5, 4, 3, 2, 1, or 0 amino acid residues and a VL region differing from the amino acid sequence of the VL region of 8B5 by no more than 10, 9, 8, 7, 6, 5, 4, 3, 2, 1, or 0 amino acid residues;
(d) a VH region differing from the amino acid sequence of the VH region of 4E9 by no more than 10, 9, 8, 7, 6, 5, 4, 3, 2, 1, or 0 amino acid residues and a VL region differing from the amino acid sequence of the VL region of 4E9 by no more than 10, 9, 8, 7, 6, 5, 4, 3, 2, 1, or 0 amino acid residues; and
(e) a VH region differing from the amino acid sequence of the VH region of 11F11 by no more than 10, 9, 8, 7, 6, 5, 4, 3, 2, 1, or 0 amino acid residues and a VL region differing from the amino acid sequence of the VL region of 10E3 by no more than 10, 9, 8, 7, 6, 5, 4, 3, 2, 1, or 0 amino acid residues;
and wherein the VH and VL region or regions are linked by at least one linker.

In other embodiments, the invention relates to antigen binding molecules (and chimeric antigen receptors comprising these molecules) wherein the linker comprises at least one of the scFv G4S linker and the scFv Whitlow linker.

In other embodiments, the invention relates to vectors encoding the polypeptides of the invention and to immune cells comprising these polypeptides. Preferred immune cells include T cells, tumor infiltrating lymphocytes (TILs), NK cells, TCR-expressing cells, dendritic cells, or NK-T cells. The T cells may be autologous, allogeneic, or heterologous.

In other embodiments, the invention relates to isolated polynucleotides encoding a chimeric antigen receptor (CAR) or T cell receptor (TCR) comprising an antigen binding molecule that specifically binds to FLT3, wherein the antigen binding molecule comprises a variable heavy (VH) chain CDR3 comprising an amino acid sequence of SEQ ID NO: 19 or SEQ ID NO:27. The polynucleotides may further comprise an activating domain. In preferred embodiments, the activating domain is CD3, more preferably CD3 zeta, more preferably the amino acid sequence set forth in SEQ ID NO: 9.

In other embodiments, the invention includes a costimulatory domain, such as CD28, CD28T, OX40, 4-1BB/CD137, CD2, CD3 (alpha, beta, delta, epsilon, gamma, zeta), CD4, CDS, CD7, CD9, CD16, CD22, CD27, CD30, CD 33, CD37, CD40, CD 45, CD64, CD80, CD86, CD134, CD137, CD154, PD-1, ICOS, lymphocyte function-associated antigen-1 (LFA-1 (CD11a/CD18), CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNF, TNFr, integrin, signaling lymphocytic activation molecule, BTLA, Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CDl-ld, ITGAE, CD103, ITGAL, CDl-la, LFA-1, ITGAM, CDl-lb, ITGAX, CDl-lc, ITGBl, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, or fragments or combinations thereof. Preferred costimulatory domains are recited hereinbelow.

In further embodiments, the invention relates to isolated polynucleotides encoding a chimeric antigen receptor (CAR) or T cell receptor (TCR), wherein said CAR or TCR comprises an antigen binding molecule that specifically binds to FLT3, and wherein the antigen binding molecule comprises a variable light (VL) chain CDR3 comprising an amino acid sequence selected from SEQ ID NO:24 and SEQ ID NO:32. The polynucleotide can further comprise an activating domain. The polynucleotide can further comprise a costimulatory domain.

In other embodiments, the invention relates to isolated polynucleotides encoding a chimeric antigen receptor (CAR) or T cell receptor (TCR) comprising an antigen binding molecule that specifically binds to FLT3, wherein the antigen binding molecule heavy chain comprises CDR1 (SEQ ID NO: 17), CDR2 (SEQ ID NO: 18), and CDR3 (SEQ ID NO: 19) and the antigen binding molecule light chain comprises CDR1 (SEQ ID NO: 22), CDR2 (SEQ ID NO: 23), and CDR3 (SEQ ID NO: 24).

In other embodiments, the invention relates to isolated polynucleotides encoding a chimeric antigen receptor (CAR) or T cell receptor (TCR) comprising an antigen binding molecule that specifically binds to FLT3, wherein the antigen binding molecule heavy chain comprises CDR1 (SEQ ID NO: 17), CDR2 (SEQ ID NO: 26), and CDR3 (SEQ ID NO:27) and the antigen binding molecule light chain comprises CDR1 (SEQ ID NO: 30), CDR2 (SEQ ID NO:31), and CDR3 (SEQ ID NO:32).

The invention further relates to antigen binding molecules to FLT3 comprising at least one variable heavy chain CDR3 or variable light chain CDR3 sequence as set forth herein. The invention further relates to antigen binding molecules to FLT3 comprising at least one variable heavy chain CDR1, CDR2, and CDR3 sequences as described herein. The invention further relates to antigen binding molecules to FLT3 comprising at least one variable light chain CDR1, CDR2, and CDR3 sequences as described herein. The invention further relates to antigen binding molecules to FLT3 comprising both variable heavy chain CDR1, CDR2, CDR3, and variable light chain CDR1, CDR2, and CDR3 sequences as described herein.

Additional heavy and light chain variable domains and CDR polynuelcotide and amino acid sequences suitable for use in FLT3-binding molecules according to the present invention are found in U.S. Provisional Application Number 62/199,944, filed on Jul. 31, 2015.

The invention further relates to methods of treating a disease or disorder in a subject in need thereof comprising administering to the subject the antigen binding molecules, the CARs, TCRs, polynucleotides, vectors, cells, or compositions according to the invention. Suitable diseases for treatment include, but are not limited to, acute myeloid leukemia (AML), chronic myelogenous leukemia (CML), chronic myelomonocytic leukemia (CMML), juvenile myelomonocytic leukemia, atypical chronic myeloid leukemia, acute promyelocytic leukemia (APL), acute monoblastic leukemia, acute erythroid leukemia, acute megakaryoblastic leukemia, myelodysplastic syndrome (MDS), myeloproliferative disorder, myeloid neoplasm, myeloid sarcoma), or combinations thereof. Additional diseases include inflammatory and/or autoimmune diseases such as rheumatoid arthritis, psoriasis, allergies, asthma, Crohn's disease, IBD, IBS, fibromyalga, mastocytosis, and Celiac disease.

### BRIEF DESCRIPTION OF THE FIGURES

FIG. 1, depicts flow cytometric analysis of FLT3 cell surface expression on human cell lines.

FIG. 2, depicts CAR expression in primary human T cells electroporated with mRNA encoding for various CARs.

FIG. 3, depicts cytolytic activity of electroporated CART cells against multiple cell lines following 16 hours of coculture.

FIG. 4, comprising of FIGS. 3A, and 3B, depicts IFNγ, IL-2, and TNFα production by electroporated CAR T cells following 16 hours of coculture with the indicated target cell lines.

FIG. 5, depicts CAR expression in lentivirus transduced primary human T cells from two healthy donors.

FIG. 6, depicts the average cytolytic activity over time from two healthy donors expressing the indicated CARs cocultured with various target cell lines.

FIG. 7, comprising of FIGS. 7A, 7B and 7C, depicts IFNγ, TNFα, and IL-2 production by lentivirus transduced CAR T cells from two healthy donors following 16 hours of coculture with the indicated target cell lines.

FIG. 8, depicts proliferation of CFSE-labeled lentivirus transduced CAR T cells from two healthy donors following 5 days of coculture with CD3-CD28 beads or the indicated target cell lines.

FIG. 9, depicts CAR expression in lentivirus transduced primary human T cells used for in vivo studies.

FIG. 10, depicts bioluminescence imaging of labeled acute myeloid leukemia cells following intra-venous injection of CAR T cells in a xenogeneic model.

FIG. 11, depicts survival curves of mice injected with CAR T cells.

FIG. 12, depicts the pGAR vector map.

### DETAILED DESCRIPTION OF THE INVENTION

It will be appreciated that chimeric antigen receptors (CARs or CAR-Ts) and T cell receptors (TCRs) are genetically engineered receptors. These engineered receptors can be readily inserted into and expressed by immune cells, including T cells in accordance with techniques known in the art. With a CAR, a single receptor can be programmed to both recognize a specific antigen and, when bound to that antigen, activate the immune cell to attack and destroy the cell bearing that antigen. When these antigens exist on tumor cells, an immune cell that expresses the CAR can target and kill the tumor cell.

CARs can be engineered to bind to an antigen (such as a cell-surface antigen) by incorporating an antigen binding molecule that interacts with that targeted antigen. Preferably, the antigen binding molecule is an antibody fragment thereof, and more preferably one or more single chain antibody fragment (“scFv”). An scFv is a single chain antibody fragment having the variable regions of the heavy and light chains of an antibody linked together. See U.S. Pat. Nos. 7,741,465, and 6,319,494 as well as Eshhar et al., Cancer Immunol Immunotherapy (1997) 45: 131-136. An scFv retains the parent antibody's ability to specifically interact with target antigen. scFvs are preferred for use in chimeric antigen receptors because they can be engineered to be expressed as part of a single chain along with the other CAR components. Id. See also Krause et al., J. Exp. Med., Volume 188, No. 4, 1998 (619-626); Finney et al., Journal of Immunology, 1998, 161: 2791-2797. It will be appreciated that the antigen binding molecule is typically contained within the extracellular portion of the CAR such that it is capable of recognizing and binding to the antigen of interest. Bispecific and multispecific CARs are contemplated within the scope of the invention, with specificity to more than one target of interest.

Costimulatory Domains. Chimeric antigen receptors may incorporate costimulatory (signaling) domains to increase their potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Annu. Rev. Pharmacol. Toxicol. 56:59-83 (2016). For example, CD28 is a costimulatory protein found naturally on T-cells. The complete native amino acid sequence of CD28 is described in NCBI Reference Sequence: NP_006130.1. The complete native CD28 nucleic acid sequence is described in NCBI Reference Sequence: NM_006139.1.

Certain CD28 domains have been used in chimeric antigen receptors. In accordance with the invention, it has now been found that a novel CD28 extracellular domain, termed “CD28T”, unexpectedly provides certain benefits when utilized in a CAR construct.

The nucleotide sequence of the CD28T molecule, including the extracellular
CD28T domain, and the CD28 transmembrane and intracellular domains is set forth in SEQ
ID NO: 1:
CTTGATAATGAAAAGTCAAACGGAACAATCATTCACGTGAAGGGCAA
GCACCTCTGTCCGTCACCCTTGTTCCCTGGTCCATCCAAGCCATTCTGGGTGTTGG
TCGTAGTGGGTGGAGTCCTCGCTTGTTACTCTCTGCTCGTCACCGTGGCTTTTATA
ATCTTCTGGGTTAGATCCAAAAGAAGCCGCCTGCTCCATAGCGATTACATGAATA
TGACTCCACGCCGCCCTGGCCCCACAAGGAAACACTACCAGCCTTACGCACCAC
CTAGAGATTTCGCTGCCTATCGGAGC
The corresponding amino acid sequence is set forth in SEQ ID NO: 2:
LDNEKSNGTIIHVKGKHLCPSPLFPGPSKPFWVLVVVGGVLACYSLLVTV
AFIIFWVRSK RSRLLHSDYM NMTPRRPGPT RKHYQPYAPP RDF AAYRS
The nucleotide sequence of the extracellular portion of CD28T is set forth in SEQ
ID NO: 3:
CTTGATAATGAAAAGTCAAACGGAACAATCATTCACGTGAAGGGCAA
GCACCTCTGTCCGTCACCCTTGTTCCCTGGTCCATCCAAGCCA
The corresponding amino acid sequence of the CD28T extracellular domain is set
forth in SEQ ID NO: 4:
LDNEKSNGTI IHVKGKHLCP SPLFPGPSKP
The nucleotide sequence of the CD28 transmembrane domain is set forth in SEQ
ID NO: 5):
TTCTGGGTGTTGGTCGTAGTGGGTGGAGTCCTCGCTTGTTACTCTCTGC
TCGTCACCGTGGCTTTTATAATCTTCTGGGTT
The amino acid sequence of the CD28 transmembrane domain is set forth in
SEQ ID NO: 6:
FWVLVVVGGV LACYSLLVTV AFIIFWV
The nucleotide sequence of the CD28 intracellular signaling domain is set forth in
SEQ ID NO: 7:
AGATCCAAAAGAAGCCGCCTGCTCCATAGCGATTACATGAATATGACT
CCACGCCGCCCTGGCCCCACAAGGAAACACTACCAGCCTTACGCACCACCTAGA
GATTTCGCTGCCTATCGGAGC
The amino acid sequence of the CD28 intracellular signaling domain is set forth in
SEQ ID NO: 8:
RSKRSRLLHSDYMNMTPRRPGPTRKHYQPYAPPRDFAAYRS
Additional CD28 sequences suitable for use in the invention include the CD28
nucleotide sequence set forth in SEQ ID NO: 11:
ATTGAGGTGATGTATCCACCGCCTTACCTGGATAACGAAAAGAGTAAC
GGTACCATCATTCACGTGAAAGGTAAACACCTGTGTCCTTCTCCCCTCTTCCCCG
GGCCATCAAAGCCC
The corresponding amino acid sequence is set forth in SEQ ID NO: 12:
IEVMYPPPYLDNEKSNGTIIHVKGKHLCPSPLFPGPSKP
Other suitable extracellular or transmembrane sequences can be derived from CD8.
The nucleotide sequence of a suitable CD 8 extracellular and transmembrane domain
is set forth in SEQ ID NO: 13:
GCTGCAGCATTGAGCAACTCAATAATGTATTTTAGTCACTTTGTACCAG
TGTTCTTGCCGGCTAAGCCTACTACCACACCCGCTCCACGGCCACCTACCCCAGC
TCCTACCATCGCTTCACAGCCTCTGTCCCTGCGCCCAGAGGCTTGCCGACCGGCC
GCAGGGGGCGCTGTTCATACCAGAGGACTGGATTTCGCCTGCGATATCTATATCT
GGGCACCCCTGGCCGGAACCTGCGGCGTACTCCTGCTGTCCCTGGTCATCACGCT
CTATTGTAATCACAGGAAC
The corresponding amino acid sequence is set forth in SEQ ID NO: 14:
AAALSNSIMYFSHFVPVFLPAKPTTTPAPRPPTPAPTIASQPLSLRPEACRPA
AGGAVHTRGLDFACDIYIWAPLAGTCGVLLLSLVITLYCNHRN

Suitable costimulatory domains within the scope of the invention can be derived from, among other sources, CD28, CD28T, OX40, 4-1BB/CD137, CD2, CD3 (alpha, beta, delta, epsilon, gamma, zeta), CD4, CD5, CD7, CD9, CD16, CD22, CD27, CD30, CD 33, CD37, CD40, CD 45, CD64, CD80, CD86, CD134, CD137, CD154, PD-1, ICOS, lymphocyte function-associated antigen-1 (LFA-1 (CDlla/CD18), CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNF, TNFr, integrin, signaling lymphocytic activation molecule, BTLA, Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, TA4, CD49D, TTGA6, VLA-6, CD49f, TTGAD, CDl-ld, ITGAE, CD103, ITGAL, CDl-la, LFA-1, 1TGAM, CDl-lb, ITGAX, CDl-lc, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, or fragments or combinations thereof.

Activating Domains.

CD3 is an element of the T cell receptor on native T cells, and has been shown to
be an important intracellular activating element in CARs. In a preferred embodiment,
the CD3 is CD3 zeta, the nucleotide sequence of which is set forth in SEQ ID NO: 9:
AGGGTGAAGTTTTCCAGATCTGCAGATGCACCAGCGTATCAGCAGGGC
CAGAACCAACTGTATAACGAGCTCAACCTGGGACGCAGGGAAGAGTATGACGTT
TTGGACAAGCGCAGAGGACGGGACCCTGAGATGGGTGGCAAACCAAGACGAAA
AAACCCCCAGGAGGGTCTCTATAATGAGCTGCAGAAGGATAAGATGGCTGAAGC
CTATTCTGAAATAGGCATGAAAGGAGAGCGGAGAAGGGGAAAAGGGCACGACG
GTTTGTACCAGGGACTCAGCACTGCTACGAAGGATACTTATGACGCTCTCCACAT
GCAAGCCCTGCCACCTAGG
The corresponding amino acid of intracellular CD3 zeta is set forth in
SEQ ID NO: 10:
VKFSRSADAPAYQQGQNQLYNELNLGRREEYDVLDKRRGRDPEMGGK
PR
RKNPQEGLYNELQKDKMAEAYSEIGMKGERRRGKGHDGLYQGLSTATKDTYDALH
MQALPPR

### Domain Orientation

Structurally, it will appreciated that these domains correspond to locations relative to the immune cell. Thus, these domains can be part of the (i) “hinge” or extracellular (EC) domain (EC), (ii) the transmembrane (TM) domain, and/or (iii) the intracellular (cytoplasmic) domain (IC). The intracellular component frequently comprises in part a member of the CD3 family, preferably CD3 zeta, which is capable of activating the T cell upon binding of the antigen binding molecule to its target. In one embodiment, the hinge domain is typically comprised of at least one costimulatory domain as defined herein.

It will also be appreciated that the hinge region may also contain some or all of a member of the immunoglobulin family such as IgG1, IgG2, IgG3, IgG4, IgA, IgD, IgE, IgM, or fragment thereof.

Exemplary CAR constructs in accordance with the invention are set forth in Table 1.

TABLE 1

| Construct Name | scFv | Costimulatory Domain | Activating Domain |
|---|---|---|---|
| 24C1 CD28T | 24C1 | CD28T | CD3 zeta |
| 24C1 CD28 | 24C1 | CD28 | CD3 zeta |
| 24C1 CD8 | 24C1 | CD8 | CD3 zeta |
| 24C8 CD28T | 24C8 | CD28T | CD3 zeta |
| 24C8 CD28 | 24C8 | CD28 | CD3 zeta |
| 24C8 CD8 | 24C8 | CD8 | CD3 zeta |
| 20C5.1 CD28T | 20C5.1 | CD28T | CD3 zeta |
| 20C5.1 CD28 | 20C5.1 | CD28 | CD3 zeta |
| 20C5.1 CD8 | 20C5.1 | CD8 | CD3 zeta |
| 20C5.2 CD28T | 20C5.2 | CD28T | CD3 zeta |
| 20C5.2 CD28 | 20C5.2 | CD28 | CD3 zeta |
| 20C5.2 CD8 | 20C5.2 | CD8 | CD3 zeta |

### Domains Relative to the Cell

It will be appreciated that relative to the cell bearing the receptor, the engineered T cells of the invention comprise an antigen binding molecule (such as an scFv), an extracellular domain (which may comprise a “hinge” domain), a transmembrane domain, and an intracellular domain. The intracellular domain comprises at least in part an activating domain, preferably comprised of a CD3 family member such as CD3 zeta, CD3 epsilon, CD3 gamma, or portions thereof. It will further be appreciated that the antigen binding molecule (e.g., one or more scFvs) is engineered such that it is located in the extracellular portion of the molecule/construct, such that it is capable of recognizing and binding to its target or targets.

Extracellular Domain. The extracellular domain is beneficial for signaling and for an efficient response of lymphocytes to an antigen. Extracellular domains of particular use in this invention may be derived from (i.e., comprise) CD28, CD28T, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CDl-la/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CDlld, 1TGAE, CD103, ITGAL, CDlla, LFA-1, 1TGAM, CDllb, ITGAX, CDllc, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof. The extracellular domain may be derived either from a natural or from a synthetic source.

As described herein, extracellular domains often comprise a hinge portion. This is a portion of the extracellular domain, sometimes referred to as a “spacer” region. A variety of hinges can be employed in accordance with the invention, including costimulatory molecules as discussed above, as well as immunoglobulin (Ig) sequences or other suitable molecules to achieve the desired special distance from the target cell. In some embodiments, the entire extracellular region comprises a hinge region. In some embodiments, the hinge region comprises CD28T, or the EC domain of CD28.

Transmembrane Domain. The CAR can be designed to comprise a transmembrane domain that is fused to the extracellular domain of the CAR. It can similarly be fused to the intracellular domain of the CAR. In one embodiment, the transmembrane domain that naturally is associated with one of the domains in a CAR is used. In some instances, the transmembrane domain can be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention may be derived from (i.e. comprise) CD28, CD28T, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CDl-la/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, G1TR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CDlld, ITGAE, CD103, ITGAL, CDlla, LFA-1, ITGAM, CDllb, ITGAX, CDllc, ITGBl, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

Optionally, short linkers may form linkages between any or some of the extracellular, transmembrane, and intracellular domains of the CAR.

In one embodiment, the transmembrane domain in the CAR of the invention is a CD8 transmembrane domain. In one embodiment, the CD8 transmembrane domain comprises the transmembrane portion of the nucleic acid sequence of SEQ ID NO: 13. In another embodiment, the CD8 transmembrane domain comprises the nucleic acid sequence that encodes the transmembrane amino acid sequence contained within SEQ ID NO: 14.

In certain embodiments, the transmembrane domain in the CAR of the invention is the CD28 transmembrane domain. In one embodiment, the CD28 transmembrane domain comprises the nucleic acid sequence of SEQ ID NO: 5. In one embodiment, the CD28 transmembrane domain comprises the nucleic acid sequence that encodes the amino acid sequence of SEQ ID NO: 6. In another embodiment, the CD28 transmembrane domain comprises the amino acid sequence of SEQ ID NO: 6.

Intracellular (Cytoplasmic) Domain. The intracellular (cytoplasmic) domain of the engineered T cells of the invention can provide activation of at least one of the normal effector functions of the immune cell. Effector function of a T cell, for example, may be cytolytic activity or helper activity including the secretion of cytokines.

It will be appreciated that suitable intracellular molecules include (i.e., comprise), but are not limited to CD28, CD28T, OX-40, 4-1BB/CD137, CD2, CD7, CD27, CD30, CD40, programmed death-1 (PD-1), inducible T cell costimulator (1COS), lymphocyte function-associated antigen-1 (LFA-1, CDl-la/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CDlld, ITGAE, CD103, ITGAL, CDlla, LFA-1, ITGAM, CDllb, ITGAX, CDllc, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

In a preferred embodiment, the cytoplasmic domain of the CAR can be designed to comprise the CD3 zeta signaling domain by itself or combined with any other desired cytoplasmic domain(s) useful in the context of the CAR of the invention. For example, the cytoplasmic domain of the CAR can comprise a CD3 zeta chain portion and a costimulatory signaling region.

The cytoplasmic signaling sequences within the cytoplasmic signaling portion of the CAR of the invention may be linked to each other in a random or specified order.

In one preferred embodiment, the cytoplasmic domain is designed to comprise the signaling domain of CD3 zeta and the signaling domain of CD28. In another embodiment, the cytoplasmic domain is designed to comprise the signaling domain of CD3 zeta and the signaling domain of 4-1BB. In another embodiment, the cytoplasmic domain in the CAR of the invention is designed to comprise a portion of CD28 and CD3 zeta, wherein the cytoplasmic CD28 comprises the nucleic acid sequence set forth in SEQ ID NO: 7 and the amino acid sequence set forth in SEQ ID NO: 8. The CD3 zeta nucleic acid sequence is set forth in SEQ ID NO: 9, and the amino acid sequence is set forth in SEQ ID NO: 8.

It will be appreciated that one preferred orientation of the CARs in accordance with the invention comprises an antigen binding domain (such as scFv) in tandem with a costimulatory domain and an activating domain. The costimulatory domain can comprise one or more of an extracellular portion, a transmembrane portion, and an intracellular portion. It will be further appreciated that multiple costimulatory domains can be utilized in tandem.

In some embodiments, nucleic acids are provided comprising a promoter operably linked to a first polynucleotide encoding an antigen binding molecule, at least one costimulatory molecule, and an activating domain.

In some embodiments, the nucleic acid construct is contained within a viral vector. In some embodiments, the viral vector is selected from the group consisting of retroviral vectors, murine leukemia virus vectors, SFG vectors, adenoviral vectors, lentiviral vectors, adeno-associated virus (AAV) vectors, Herpes virus vectors, and vaccinia virus vectors. In some embodiments, the nucleic acid is contained within a plasmid.

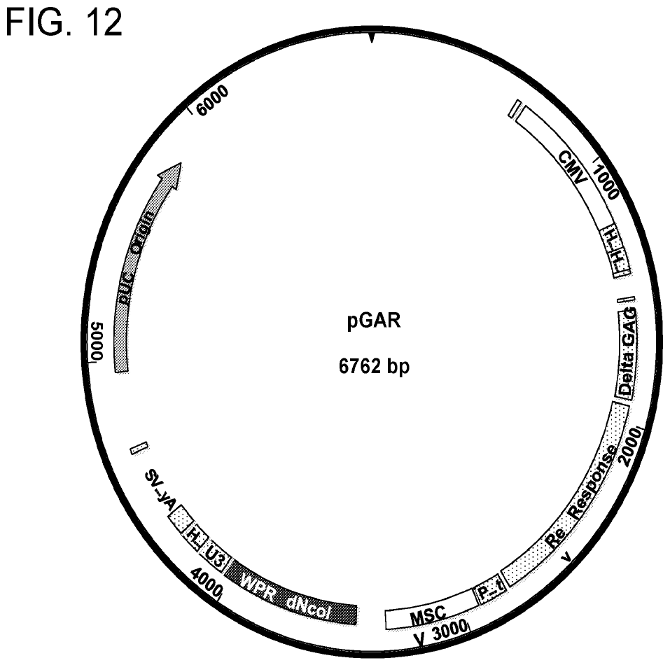

The invention further relates to isolated polynucleotides encoding the chimeric antigen receptors, and vectors comprising the polynucleotides. Any vector known in the art can be suitable for the present invention. In some embodiments, the vector is a viral vector. In some embodiments, the vector is a retroviral vector (such as pMSVG1), a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector (such as pGAR), or any combination thereof. The pGAR vector map is shown in FIG. 12. The pGAR sequence is as follows:

(SEQ ID NO: 95)
CTGACGCGCCCTGTAGCGGCGCATTAAGCGCGGCGGGTGTGGTGGTTAC
GCGCAGCGTGACCGCTACACTTGCCAGCGCCCTAGCGCCCGCTCCTTTC
GCTTTCTTCCCTTCCTTTCTCGCCACGTTCGCCGGCTTTCCCCGTCAAG
CTCTAAATCGGGGGCTCCCTTTAGGGTTCCGATTTAGTGCTTTACGGCA
CCTCGACCCCAAAAAACTTGATTAGGGTGATGGTTCACGTAGTGGGCCA
TCGCCCTGATAGACGGTTTTTCGCCCTTTGACGTTGGAGTCCACGTTCT
TTAATAGTGGACTCTTGTTCCAAACTGGAACAACACTCAACCCTATCTC
GGTCTATTCTTTTGATTTATAAGGGATTTTGCCGATTTCGGCCTATTGG
TTAAAAAATGAGCTGATTTAACAAAAATTTAACGCGAATTTTAACAAAA
TATTAACGCTTACAATTTGCCATTCGCCATTCAGGCTGCGCAACTGTTG
GGAAGGGCGATCGGTGCGGGCCTCTTCGCTATTACGCCAGCTGGCGAAA
GGGGGATGTGCTGCAAGGCGATTAAGTTGGGTAACGCCAGGGTTTTCCC
AGTCACGACGTTGTAAAACGACGGCCAGTGAATTGTAATACGACTCACT
ATAGGGCGACCCGGGGATGGCGCGCCAGTAATCAATTACGGGGTCATTA
GTTCATAGCCCATATATGGAGTTCCGCGTTACATAACTTACGGTAAATG
GCCCGCCTGGCTGACCGCCCAACGACCCCCGCCCATTGACGTCAATAAT
GACGTATGTTCCCATAGTAACGCCAATAGGGACTTTCCATTGACGTCAA
TGGGTGGAGTATTTACGGTAAACTGCCCACTTGGCAGTACATCAAGTGT
ATCATATGCCAAGTACGCCCCCTATTGACGTCAATGACGGTAAATGGCC
CGCCTGGCATTATGCCCAGTACATGACCTTATGGGACTTTCCTACTTGG
CAGTACATCTACGTATTAGTCATCGCTATTACCATGCTGATGCGGTTTT
GGCAGTACATCAATGGGCGTGGATAGCGGTTTGACTCACGGGGATTTCC
AAGTCTCCACCCCATTGACGTCAATGGGAGTTTGTTTTGGCACCAAAAT
CAACGGGACTTTCCAAAATGTCGTAACAACTCCGCCCCATTGACGCAAA
TGGGCGGTAGGCGTGTACGGTGGGAGGTCTATATAAGCAGAGCTGGTTT
AGTGAACCGGGGTCTCTCTGGTTAGACCAGATCTGAGCCTGGGAGCTCT
CTGGCTAACTAGGGAACCCACTGCTTAAGCCTCAATAAAGCTTGCCTTG
AGTGCTTCAAGTAGTGTGTGCCCGTCTGTTGTGTGACTCTGGTAACTAG
AGATCCCTCAGACCCTTTTAGTCAGTGTGGAAAATCTCTAGCAGTGGCG
CCCGAACAGGGACTTGAAAGCGAAAGGGAAACCAGAGGAGCTCTCTCGA
CGCAGGACTCGGCTTGCTGAAGCGCGCACGGCAAGAGGCGAGGGGCGGC
GACTGGTGAGTACGCCAAAAATTTTGACTAGCGGAGGCTAGAAGGAGAG
AGATGGGTGCGAGAGCGTCAGTATTAAGCGGGGGAGAATTAGATCGCGA
TGGGAAAAAATTCGGTTAAGGCCAGGGGGAAAGAAAAAATATAAATTAA
AACATATAGTATGGGCAAGCAGGGAGCTAGAACGATTCGCAGTTAATCC
TGGCCTGTTAGAAACATCAGAAGGCTGTAGACAAATACTGGGACAGCTA
CAACCATCCCTTCAGACAGGATCAGAAGAACTTAGATCATTATATAATA
CAGTAGCAACCCTCTATTGTGTGCATCAAAGGATAGAGATAAAAGACAC
CAAGGAAGCTTTAGACAAGATAGAGGAAGAGCAAAACAAAAGTAAGACC
ACCGCACAGCAAGCCGCCGCTGATCTTCAGACCTGGAGGAGGAGATATG
AGGGACAATTGGAGAAGTGAATTATATAAATATAAAGTAGTAAAAATTG
AACCATTAGGAGTAGCACCCACCAAGGCAAAGAGAAGAGTGGTGCAGAG
AGAAAAAAGAGCAGTGGGAATAGGAGCTTTGTTCCTTGGGTTCTTGGGA
GCAGCAGGAAGCACTATGGGCGCAGCGTCAATGACGCTGACGGTACAGG
CCAGACAATTATTGTCTGGTATAGTGCAGCAGCAGAACAATTTGCTGAG
GGCTATTGAGGCGCAACAGCATCTGTTGCAACTCACAGTCTGGGGCATC
AAGCAGCTCCAGGCAAGAATCCTGGCTGTGGAAAGATACCTAAAGGATC
AACAGCTCCTGGGGATTTGGGGTTGCTCTGGAAAACTCATTTGCACCAC
TGCTGTGCCTTGGAATGCTAGTTGGAGTAATAAATCTCTGGAACAGATT
TGGAATCACACGACCTGGATGGAGTGGGACAGAGAAATTAACAATTACA
CAAGCTTAATACACTCCTTAATTGAAGAATCGCAAAACCAGCAAGAAAA
GAATGAACAAGAATTATTGGAATTAGATAAATGGGCAAGTTTGTGGAAT
TGGTTTAACATAACAAATTGGCTGTGGTATATAAAATTATTCATAATGA
TAGTAGGAGGCTTGGTAGGTTTAAGAATAGTTTTTGCTGTACTTTCTAT
AGTGAATAGAGTTAGGCAGGGATATTCACCATTATCGTTTCAGACCCAC
CTCCCAACCCCGAGGGGACCCGACAGGCCCGAAGGAATAGAAGAAGAAG
GTGGAGAGAGAGACAGAGACAGATCCATTCGATTAGTGAACGGATCTCG
ACGGTATCGGTTAACTTTTAAAAGAAAAGGGGGGATTGGGGGGTACAGT
GCAGGGGAAAGAATAGTAGACATAATAGCAACAGACATACAAACTAAAG
AATTACAAAAACAAATTACAAAATTCAAAATTTTATCGCGATCGCGGAA
TGAAAGACCCCACCTGTAGGTTTGGCAAGCTAGCTTAAGTAACGCCATT
TTGCAAGGCATGGAAAATACATAACTGAGAATAGAGAAGTTCAGATCAA
GGTTAGGAACAGAGAGACAGCAGAATATGGGCCAAACAGGATATCTGTG
GTAAGCAGTTCCTGCCCCGGCTCAGGGCCAAGAACAGATGGTCCCCAGA
TGCGGTCCCGCCCTCAGCAGTTTCTAGAGAACCATCAGATGTTTCCAGG
GTGCCCCAAGGACCTGAAAATGACCCTGTGCCTTATTTGAACTAACCAA
TCAGTTCGCTTCTCGCTTCTGTTCGCGCGCTTCTGCTCCCCGAGCTCAA
TAAAAGAGCCCACAACCCCTCACTCGGCGCGCCAGTCCTTCGAAGTAGA
TCTTTGTCGATCCTACCATCCACTCGACACACCCGCCAGCGGCCGCTGC
CAAGCTTCCGAGCTCTCGAATTAATTCACGGTACCCACCATGGCCTAGG
GAGACTAGTCGAATCGATATCAACCTCTGGATTACAAAATTTGTGAAAG
ATTGACTGGTATTCTTAACTATGTTGCTCCTTTTACGCTATGTGGATAC
GCTGCTTTAATGCCTTTGTATCATGCTATTGCTTCCCGTATGGCTTTCA
TTTTCTCCTCCTTGTATAAATCCTGGTTGCTGTCTCTTTATGAGGAGTT
GTGGCCCGTTGTCAGGCAACGTGGCGTGGTGTGCACTGTGTTTGCTGAC
GCAACCCCCACTGGTTGGGGCATTGCCACCACCTGTCAGCTCCTTTCCG
GGACTTTCGCTTTCCCCCTCCCTATTGCCACGGCGGAACTCATCGCCGC
CTGCCTTGCCCGCTGCTGGACAGGGGCTCGGCTGTTGGGCACTGACAAT
TCCGTGGTGTTGTCGGGGAAGCTGACGTCCTTTTCATGGCTGCTCGCCT
GTGTTGCCACCTGGATTCTGCGCGGGACGTCCTTCTGCTACGTCCCTTC
GGCCCTCAATCCAGCGGACCTTCCTTCCCGCGGCCTGCTGCCGGCTCTG
CGGCCTCTTCCGCGTCTTCGCCTTCGCCCTCAGACGAGTCGGATCTCCC
TTTGGGCCGCCTCCCCGCCTGGTTAATTAAAGTACCTTTAAGACCAATG
ACTTACAAGGCAGCTGTAGATCTTAGCCACTTTTTAAAAGAAAAGGGGG
GACTGGAAGGGCGAATTCACTCCCAACGAAGACAAGATCTGCTTTTTGC
TTGTACTGGGTCTCTCTGGTTAGACCAGATCTGAGCCTGGGAGCTCTCT
GGCTAACTAGGGAACCCACTGCTTAAGCCTCAATAAAGCTTGCCTTGAG
TGCTTCAAGTAGTGTGTGCCCGTCTGTTGTGTGACTCTGGTAACTAGAG
ATCCCTCAGACCCTTTTAGTCAGTGTGGAAAATCTCTAGCAGGCATGCC
AGACATGATAAGATACATTGATGAGTTTGGACAAACCACAACTAGAATG
CAGTGAAAAAAATGCTTTATTTGTGAAATTTGTGATGCTATTGCTTTAT
TTGTAACCATTATAAGCTGCAATAAACAAGTTAACAACAACAATTGCAT
TCATTTTATGTTTCAGGTTCAGGGGGAGGTGTGGGAGGTTTTTTGGCGC
GCCATCGTCGAGGTTCCCTTTAGTGAGGGTTAATTGCGAGCTTGGCGTA
ATCATGGTCATAGCTGTTTCCTGTGTGAAATTGTTATCCGCTCACAATT
CCACACAACATACGAGCCGGAAGCATAAAGTGTAAAGCCTGGGGTGCCT
AATGAGTGAGCTAACTCACATTAATTGCGTTGCGCTCACTGCCCGCTTT
CCAGTCGGGAAACCTGTCGTGCCAGCTGCATTAATGAATCGGCCAACGC
GCGGGGAGAGGCGGTTTGCGTATTGGGCGCTCTTCCGCTTCCTCGCTCA
CTGACTCGCTGCGCTCGGTCGTTCGGCTGCGGCGAGCGGTATCAGCTCA
CTCAAAGGCGGTAATACGGTTATCCACAGAATCAGGGGATAACGCAGGA
AAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCA
CAAAAATCGACGCTCAAGTCAGAGGTGGCGAAACCCGACAGGACTATAA
AGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAG
CGTGGCGCTTTCTCATAGCTCACGCTGTAGGTATCTCAGTTCGGTGTAG
GTCGTTCGCTCCAAGCTGGGCTGTGTGCACGAACCCCCCGTTCAGCCCG
ACCGCTGCGCCTTATCCGGTAACTATCGTCTTGAGTCCAACCCGGTAAG
ACACGACTTATCGCCACTGGCAGCAGCCACTGGTAACAGGATTAGCAGA
GCGAGGTATGTAGGCGGTGCTACAGAGTTCTTGAAGTGGTGGCCTAACT
ACGGCTACACTAGAAGAACAGTATTTGGTATCTGCGCTCTGCTGAAGCC
AGTTACCTTCGGAAAAAGAGTTGGTAGCTCTTGATCCGGCAAACAAACC
ACCGCTGGTAGCGGTGGTTTTTTTGTTTGCAAGCAGCAGATTACGCGCA
GAAAAAAAGGATCTCAAGAAGATCCTTTGATCTTTTCTACGGGGTCTGA
CGCTCAGTGGAACGAAAACTCACGTTAAGGGATTTTGGTCATGAGATTA
TCAAAAAGGATCTTCACCTAGATCCTTTTAAATTAAAAATGAAGTTTTA
AATCAATCTAAAGTATATATGAGTAAACTTGGTCTGACAGTTACCAATG
CTTAATCAGTGAGGCACCTATCTCAGCGATCTGTCTATTTCGTTCATCC
ATAGTTGCCTGACTCCCCGTCGTGTAGATAACTACGATACGGGAGGGCT
TACCATCTGGCCCCAGTGCTGCAATGATACCGCGAGACCCACGCTCACC
GGCTCCAGATTTATCAGCAATAAACCAGCCAGCCGGAAGGGCCGAGCGC
AGAAGTGGTCCTGCAACTTTATCCGCCTCCATCCAGTCTATTAATTGTT
GCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATG
GCTTCATTCAGCTCCGGTTCCCAACGATCAAGGCGAGTTACATGATCCC
CCATGTTGTGCAAAAAAGCGGTTAGCTCCTTCGGTCCTCCGATCGTTGT
CAGAAGTAAGTTGGCCGCAGTGTTATCACTCATGGTTATGGCAGCACTG
CATAATTCTCTTACTGTCATGCCATCCGTAAGATGCTTTTCTGTGACTG
GTGAGTACTCAACCAAGTCATTCTGAGAATAGTGTATGCGGCGACCGAG
TTGCTCTTGCCCGGCGTCAATACGGGATAATACCGCGCCACATAGCAGA
ACTTTAAAAGTGCTCATCATTGGAAAACGTTCTTCGGGGCGAAAACTCT
CAAGGATCTTACCGCTGTTGAGATCCAGTTCGATGTAACCCACTCGTGC
ACCCAACTGATCTTCAGCATCTTTTACTTTCACCAGCGTTTCTGGGTGA
GCAAAAACAGGAAGGCAAAATGCCGCAAAAAAGGGAATAAGGGCGACAC
GGAAATGTTGAATACTCATACTCTTCCTTTTTCAATATTATTGAAGCAT
TTATCAGGGTTATTGTCTCATGAGCGGATACATATTTGAATGTATTTAG
AAAAATAAACAAATAGGGGTTCCGCGCACATTTCCCCGAAAAGTGCCAC

Suitable additional exemplary vectors include e.g., pBABE-puro, pBABE-neo largeTcDNA, pBABE-hygro-hTERT, pMKO.1 GFP, MSCV-IRES-GFP, pMSCV PIG (Puro IRES GFP empty plasmid), pMSCV-loxp-dsRed-loxp-eGFP-Puro-WPRE, MSCV IRES Luciferase, pMIG, MDH1 -PGK-GFP_2.0, TtRMPVIR, pMSCV-IRES-mCherry FP, pRetroX GFP T2A Cre, pRXTN, pLncEXP, and pLXIN-Luc.

In some embodiments, the engineered immune cell is a T cell, tumor infiltrating lymphocyte (TIL), NK cell, TCR-expressing cell, dendritic cell, or NK-T cell. In some embodiments, the cell is obtained or prepared from peripheral blood. In some embodiments, the cell is obtained or prepared from peripheral blood mononuclear cells (PBMCs). In some embodiments, the cell is obtained or prepared from bone marrow. In some embodiments, the cell is obtained or prepared from umbilical cord blood. In some embodiments, the cell is a human cell. In some embodiments, the cell is transfected or transduced by the nucleic acid vector using a method selected from the group consisting of electroporation, sonoporation, biolistics (e.g., Gene Gun), lipid transfection, polymer transfection, nanoparticles, or polyplexes.

In some embodiments, chimeric antigen receptors are expressed in the engineered immune cells that comprise the nucleic acids of the present application. These chimeric antigen receptors of the present application may comprise, in some embodiments, (i) an antigen binding molecule (such as an scFv), (ii) a transmembrane region, and (iii) a T cell activation molecule or region.

### Antigen Binding Molecules

Antigen binding molecules are within the scope of the invention.

An “antigen binding molecule” as used herein means any protein that binds a specified target antigen. In the instant application, the specified target antigen is the FLT3 protein or fragment thereof. Antigen binding molecules include, but are not limited to antibodies and binding parts thereof, such as immunologically functional fragments. Peptibodies (i.e., Fc fusion molecules comprising peptide binding domains) are another example of suitable antigen binding molecules.

In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments, the antigen binding molecule binds to FLT3. In further embodiments, the antigen binding molecule is an antibody of fragment thereof, including one or more of the complementarity determining regions (CDRs) thereof. In further embodiments, the antigen binding molecule is a single chain variable fragment (scFv).

The term “immunologically functional fragment” (or “fragment”) of an antigen binding molecule is a species of antigen binding molecule comprising a portion (regardless of how that portion is obtained or synthesized) of an antibody that lacks at least some of the amino acids present in a full-length chain but which is still capable of specifically binding to an antigen. Such fragments are biologically active in that they bind to the target antigen and can compete with other antigen binding molecules, including intact antibodies, for binding to a given epitope. In some embodiments, the fragments are neutralizing fragments. In some embodiments, the fragments can block or reduce the activity of FLT3. In one aspect, such a fragment will retain at least one CDR present in the full-length light or heavy chain, and in some embodiments will comprise a single heavy chain and/or light chain or portion thereof. These fragments can be produced by recombinant DNA techniques, or can be produced by enzymatic or chemical cleavage of antigen binding molecules, including intact antibodies.

Immunologically functional immunoglobulin fragments include, but are not limited to, scFv fragments, Fab fragments (Fab′, F(ab′)₂, and the like), one or more CDR, a diabody (heavy chain variable domain on the same polypeptide as a light chain variable domain, connected via a short peptide linker that is too short to permit pairing between the two domains on the same chain), domain antibodies, and single-chain antibodies. These fragments can be derived from any mammalian source, including but not limited to human, mouse, rat, camelid or rabbit. As will be appreciated by one of skill in the art, an antigen binding molecule can include non-protein components.

Variants of the antigen binding molecules are also within the scope of the invention, e.g., variable light and/or variable heavy chains that each have at least 70-80%, 80-85%, 85-90%, 90-95%, 95-97%, 97-99%, or above 99% identity to the amino acid sequences of the sequences described herein. In some instances, such molecules include at least one heavy chain and one light chain, whereas in other instances the variant forms contain two identical light chains and two identical heavy chains (or subparts thereof). A skilled artisan will be able to determine suitable variants of the antigen binding molecules as set forth herein using well-known techniques. In certain embodiments, one skilled in the art can identify suitable areas of the molecule that may be changed without destroying activity by targeting regions not believed to be important for activity.

In certain embodiments, the polypeptide structure of the antigen binding molecules is based on antibodies, including, but not limited to, monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the antigen binding molecule comprises or consists of avimers.

In some embodiments, an antigen binding molecule to FLT3 is administered alone. In other embodiments, the antigen binding molecule to FLT3 is administered as part of a CAR, TCR, or other immune cell. In such immune cells, the antigen binding molecule to FLT3 can be under the control of the same promoter region, or a separate promoter. In certain embodiments, the genes encoding protein agents and/or an antigen binding molecule to FLT3 can be in separate vectors.

The invention further provides for pharmaceutical compositions comprising an antigen binding molecule to FLT3 together with a pharmaceutically acceptable diluent, carrier, solubilizer, emulsifier, preservative and/or adjuvant. In certain embodiments, pharmaceutical compositions will include more than one different antigen binding molecule to FLT3. In certain embodiments, pharmaceutical compositions will include more than one antigen binding molecule to FLT3 wherein the antigen binding molecules to FLT3 bind more than one epitope. In some embodiments, the various antigen binding molecules will not compete with one another for binding to FLT3.

In other embodiments, the pharmaceutical composition can be selected for parenteral delivery, for inhalation, or for delivery through the digestive tract, such as orally. The preparation of such pharmaceutically acceptable compositions is within the ability of one skilled in the art. In certain embodiments, buffers are used to maintain the composition at physiological pH or at a slightly lower pH, typically within a pH range of from about 5 to about 8. In certain embodiments, when parenteral administration is contemplated, a therapeutic composition can be in the form of a pyrogen-free, parenterally acceptable aqueous solution comprising a desired antigen binding molecule to FLT3, with or without additional therapeutic agents, in a pharmaceutically acceptable vehicle. In certain embodiments, a vehicle for parenteral injection is sterile distilled water in which an antigen binding molecule to FLT3, with or without at least one additional therapeutic agent, is formulated as a sterile, isotonic solution, properly preserved. In certain embodiments, the preparation can involve the formulation of the desired molecule with polymeric compounds (such as polylactic acid or polyglycolic acid), beads or liposomes that can provide for the controlled or sustained release of the product which can then be delivered via a depot injection. In certain embodiments, implantable drug delivery devices can be used to introduce the desired molecule.

In some embodiments, the antigen binding molecule is used as a diagnostic or validation tool. The antigen binding molecule can be used to assay the amount of FLT3 present in a sample and/or subject. In some embodiments, the diagnostic antigen binding molecule is not neutralizing. In some embodiments, the antigen binding molecules disclosed herein are used or provided in an assay kit and/or method for the detection of FLT3 in mammalian tissues or cells in order to screen/diagnose for a disease or disorder associated with changes in levels of FLT3. The kit can comprise an antigen binding molecule that binds FLT3, along with means for indicating the binding of the antigen binding molecule with FLT3, if present, and optionally FLT3 protein levels.

The antigen binding molecules will be further understood in view of the definitions and descriptions below.

An “Fc” region comprises two heavy chain fragments comprising the CH1 and CH2 domains of an antibody. The two heavy chain fragments are held together by two or more disulfide bonds and by hydrophobic interactions of the CH3 domains.

A “Fab fragment” comprises one light chain and the CH1 and variable regions of one heavy chain. The heavy chain of a Fab molecule cannot form a disulfide bond with another heavy chain molecule. A “Fab′ fragment” comprises one light chain and a portion of one heavy chain that contains the VH domain and the CH1 domain and also the region between the CH1 and CH2 domains, such that an interchain disulfide bond can be formed between the two heavy chains of two Fab′ fragments to form an F(ab′)₂ molecule. An “F(ab′)₂ fragment” contains two light chains and two heavy chains containing a portion of the constant region between the CH1 and CH2 domains, such that an interchain disulfide bond is formed between the two heavy chains. An F(ab′)₂ fragment thus is composed of two Fab′ fragments that are held together by a disulfide bond between the two heavy chains.

The “Fv region” comprises the variable regions from both the heavy and light chains, but lacks the constant regions.

“Single chain variable fragment” (“scFv”, also termed “single-chain antibody”) refers to Fv molecules in which the heavy and light chain variable regions have been connected by a flexible linker to form a single polypeptide chain, which forms an antigen binding region. See PCT application WO88/01649 and U.S. Pat. Nos. 4,946,778 and 5,260,203, the disclosures of which are incorporated by reference in their entirety.

A “bivalent antigen binding molecule” comprises two antigen binding sites. In some instances, the two binding sites have the same antigen specificities. Bivalent antigen binding molecules can be bispecific. A “multispecific antigen binding molecule” is one that targets more than one antigen or epitope. A “bispecific,” “dual-specific” or “bifunctional” antigen binding molecule is a hybrid antigen binding molecule or antibody, respectively, having two different antigen binding sites. The two binding sites of a bispecific antigen binding molecule will bind to two different epitopes, which can reside on the same or different protein targets.

An antigen binding molecule is said to “specifically bind” its target antigen when the dissociation constant (K_{d}) is ˜1×10⁻⁷ M. The antigen binding molecule specifically binds antigen with “high affinity” when the K_{d} is 1-5×10⁻⁹ M, and with “very high affinity” when the K_{d} is 1-5×10⁻¹⁰ M. In one embodiment, the antigen binding molecule has a K_{d} of 10⁻⁹ M. In one embodiment, the off-rate is <1×10⁻⁵. In other embodiments, the antigen binding molecules will bind to human FLT3 with a K_{d} of between about 10⁻⁷ M and 10⁻¹³ M, and in yet another embodiment the antigen binding molecules will bind with a K_{d} 1.0-5×10⁻¹⁰.

An antigen binding molecule is said to be “selective” when it binds to one target more tightly than it binds to a second target.

The term “antibody” refers to an intact immunoglobulin of any isotype, or a fragment thereof that can compete with the intact antibody for specific binding to the target antigen, and includes, for instance, chimeric, humanized, fully human, and bispecific antibodies. An “antibody” is a species of an antigen binding molecule as defined herein. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be chimeric, that is, different portions of the antibody can be derived from two different antibodies as described further below. The antigen binding molecules, antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”) and fragments thereof, respectively.

The variable regions typically exhibit the same general structure of relatively conserved framework regions (FR) joined by the 3 hypervariable regions (i.e., “CDRs”). The CDRs from the two chains of each pair typically are aligned by the framework regions, which can enable binding to a specific epitope. From N-terminal to C-terminal, both light and heavy chain variable regions typically comprise the domains FRI, CDR1, FR2, CDR2, FR3, CDR3 and FR4. By convention, CDR regions in the heavy chain are typically referred to as HC CDR1, CDR2, and CDR3. The CDR regions in the light chain are typically referred to as LC CDR1, CDR2, and CDR3. The assignment of amino acids to each domain is typically in accordance with the definitions of Kabat (Seqs of Proteins of Immunological Interest (NIH, Bethesda, Md. (1987 and 1991)), or Chothia (J. Mol. Biol., 196:901-917 (1987); Chothia et al., Nature, 342:878-883 (1989)). Various methods of analysis can be employed to identify or approximate the CDR regions, including not only Kabat or Chothia, but also the AbM definition.

The term “light chain” includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, V_{H}, and three constant region domains, CH1, CH2, and CH3. The V_{H} domain is at the amino-terminus of the polypeptide, and the C_{H} domains are at the carboxyl-terminus, with the CH3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. The variable region of an antibody typically determines specificity of a particular antibody for its target.

Variability is not evenly distributed throughout the variable domains of antibodies; it is concentrated in sub-domains of each of the heavy and light chain variable regions. These subdomains are called “hypervariable regions” or “complementarity determining regions” (CDRs). The more conserved (i.e., non-hypervariable) portions of the variable domains are called the “framework” regions (FRM or FR) and provide a scaffold for the six CDRs in three dimensional space to form an antigen-binding surface. The variable domains of naturally occurring heavy and light chains each comprise four FRM regions (FR1, FR2, FR3, and FR4), largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding site (see Kabat et al., loc. cit.).

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDRH1, CDR-H2 and CDR-H3). CDRs contain most of the residues responsible for specific interactions of the antibody with the antigen and hence contribute to the functional activity of an antibody molecule: they are the main determinants of antigen specificity.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., loc. cit.; Chothia et al., J. MoI. Biol, 1987, 196: 901-917; and MacCallum et al., J. Mol. Biol, 1996, 262: 732). Still another standard for characterizing the antigen binding site is the AbM definition used by Oxford Molecular's AbM antibody modeling software. See, e.g., Protein Sequence and Structure Analysis of Antibody Variable Domains. In: Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the so-called Kabat system is preferred.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. Mol. Biol., 1987, 196: 901; Chothia et al., Nature, 1989, 342: 877; Martin and Thornton, J. Mol. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al., loc. cit.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988.

The CDR3 of the light chain and, particularly, the CDR3 of the heavy chain may constitute the most important determinants in antigen binding within the light and heavy chain variable regions. In some antibody constructs, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen. Hence, CDR3 is typically the greatest source of molecular diversity within the antibody-binding site. H3, for example, can be as short as two amino acid residues or greater than 26 amino acids.

The term “neutralizing” refers to an antigen binding molecule, scFv, or antibody, respectively, that binds to a ligand and prevents or reduces the biological effect of that ligand. This can be done, for example, by directly blocking a binding site on the ligand or by binding to the ligand and altering the ligand's ability to bind through indirect means (such as structural or energetic alterations in the ligand). In some embodiments, the term can also denote an antigen binding molecule that prevents the protein to which it is bound from performing a biological function.

The term “target” or “antigen” refers to a molecule or a portion of a molecule capable of being bound by an antigen binding molecule. In certain embodiments, a target can have one or more epitopes.

The term “compete” when used in the context of antigen binding molecules that compete for the same epitope means competition between antigen binding molecules as determined by an assay in which the antigen binding molecule (e.g., antibody or immunologically functional fragment thereof) being tested prevents or inhibits (e.g., reduces) specific binding of a reference antigen binding molecule to an antigen. Numerous types of competitive binding assays can be used to determine if one antigen binding molecule competes with another, for example: solid phase direct or indirect radioimmunoassay (RIA), solid phase direct or indirect enzyme immunoassay (EIA), sandwich competition assay (Stahli et al., 1983, Methods in Enzymology 9:242-253); solid phase direct biotin-avidin EIA (Kirkland et al., 1986, J. Immunol. 137:3614-3619), solid phase direct labeled assay, solid phase direct labeled sandwich assay (Harlow and Lane, 1988, Antibodies, A Laboratory Manual, Cold Spring Harbor Press); solid phase direct label RIA using 1-125 label (Morel et al., 1988, Molec. Immunol. 25:7-15); solid phase direct biotin-avidin EIA (Cheung, et al., 1990, Virology 176:546-552); and direct labeled RIA (Moldenhauer et al., 1990, Scand. J. Immunol. 32:77-82). The term “epitope” includes any determinant capable of being bound by an antigen binding molecule, such as an scFv, antibody, or immune cell of the invention. An epitope is a region of an antigen that is bound by an antigen binding molecule that targets that antigen, and when the antigen is a protein, includes specific amino acids that directly contact the antigen binding molecule.

As used herein, the terms “label” or “labeled” refers to incorporation of a detectable marker, e.g., by incorporation of a radiolabeled amino acid or attachment to a polypeptide of biotin moieties that can be detected by marked avidin (e.g., streptavidin containing a fluorescent marker or enzymatic activity that can be detected by optical or colorimetric methods). In certain embodiments, the label or marker can also be therapeutic. Various methods of labeling polypeptides and glycoproteins are known in the art and can be used.

In accordance with the invention, on-off or other types of control switch techniques may be incorporated herein. These techniques may employ the use of dimerization domains and optional activators of such domain dimerization. These techniques include, e.g., those described by Wu et al., Science 2014 350 (6258) utilizing FKBP/Rapalog dimerization systems in certain cells, the contents of which are incorporated by reference herein in their entirety. Additional dimerization technology is described in, e.g., Fegan et al. Chem. Rev. 2010, 110, 3315-3336 as well as U.S. Pat. Nos. 5,830,462; 5,834,266; 5,869,337; and 6,165,787, the contents of which are also incorporated by reference herein in their entirety. Additional dimerization pairs may include cyclosporine-A/cyclophilin, receptor, estrogen/estrogen receptor (optionally using tamoxifen), glucocorticoids/glucocorticoid receptor, tetracycline/tetracycline receptor, vitamin D/vitamin D receptor. Further examples of dimerization technology can be found in e.g., WO 2014/127261, WO 2015/090229, US 2014/0286987, US 2015/0266973, US 2016/0046700, U.S. Pat. No. 8,486,693, US 2014/0171649, and US 2012/0130076, the contents of which are further incorporated by reference herein in their entirety.

### Methods of Treatment

Using adoptive immunotherapy, native T cells can be (i) removed from a patient, (ii) genetically engineered to express a chimeric antigen receptor (CAR) that binds to at least one tumor antigen (iii) expanded ex vivo into a larger population of engineered T cells, and (iv) reintroduced into the patient. See e.g., U.S. Pat. Nos. 7,741,465, and 6,319,494, Eshhar et al. (Cancer Immunol, supra); Krause et al. (supra); Finney et al. (supra). After the engineered T cells are reintroduced into the patient, they mediate an immune response against cells expressing the tumor antigen. See e.g., Krause et al., J. Exp. Med., Volume 188, No. 4, 1998 (619-626). This immune response includes secretion of IL-2 and other cytokines by T cells, the clonal expansion of T cells recognizing the tumor antigen, and T cell-mediated specific killing of target-positive cells. See Hombach et al., Journal of Immun. 167: 6123-6131 (2001).

In some aspects, the invention therefore comprises a method for treating or preventing a condition associated with undesired and/or elevated FLT3 levels in a patient, comprising administering to a patient in need thereof an effective amount of at least one isolated antigen binding molecule, CAR, or TCR disclosed herein.

Methods are provided for treating diseases or disorders, including cancer. In some embodiments, the invention relates to creating a T cell-mediated immune response in a subject, comprising administering an effective amount of the engineered immune cells of the present application to the subject. In some embodiments, the T cell-mediated immune response is directed against a target cell or cells. In some embodiments, the engineered immune cell comprises a chimeric antigen receptor (CAR), or a T cell receptor (TCR). In some embodiments, the target cell is a tumor cell. In some aspects, the invention comprises a method for treating or preventing a malignancy, said method comprising administering to a subject in need thereof an effective amount of at least one isolated antigen binding molecule described herein. In some aspects, the invention comprises a method for treating or preventing a malignancy, said method comprising administering to a subject in need thereof an effective amount of at least one immune cell, wherein the immune cell comprises at least one chimeric antigen receptor, T cell receptor, and/or isolated antigen binding molecule as described herein.

In some aspects, the invention comprises a pharmaceutical composition comprising at least one antigen binding molecule as described herein and a pharmaceutically acceptable excipient. In some embodiments, the pharmaceutical composition further comprises an additional active agent.

The antigen binding molecules, CARs, TCRs, immune cells, and the like of the invention can be used to treat myeloid diseases including but not limited to acute myeloid leukemia (AML), chronic myelogenous leukemia (CML), chronic myelomonocytic leukemia (CMML), juvenile myelomonocytic leukemia, atypical chronic myeloid leukemia, acute promyelocytic leukemia (APL), acute monoblastic leukemia, acute erythroid leukemia, acute megakaryoblastic leukemia, myelodysplastic syndrome (MDS), myeloproliferative disorder, myeloid neoplasm, myeloid sarcoma), or combinations thereof Additional diseases include inflammatory and/or autoimmune diseases such as rheumatoid arthritis, psoriasis, allergies, asthma, Crohn's disease, IBD, IBS, fibromyalga, mastocytosis, and Celiac disease.

It will be appreciated that target doses for CAR⁺/CAR-T⁺/TCR⁺ cells can range from 1×10⁶-2×10¹⁰ cells/kg, preferably 2×10⁶ cells/kg, more preferably. It will be appreciated that doses above and below this range may be appropriate for certain subjects, and appropriate dose levels can be determined by the healthcare provider as needed. Additionally, multiple doses of cells can be provided in accordance with the invention.

Also provided are methods for reducing the size of a tumor in a subject, comprising administering to the subject an engineered cell of the present invention to the subject, wherein the cell comprises a chimeric antigen receptor, a T cell receptor, or a T cell receptor based chimeric antigen receptor comprising an antigen binding molecule binds to an antigen on the tumor. In some embodiments, the subject has a solid tumor, or a blood malignancy such as lymphoma or leukemia. In some embodiments, the engineered cell is delivered to a tumor bed. In some embodiments, the cancer is present in the bone marrow of the subject.

In some embodiments, the engineered cells are autologous T cells. In some embodiments, the engineered cells are allogeneic T cells. In some embodiments, the engineered cells are heterologous T cells. In some embodiments, the engineered cells of the present application are transfected or transduced in vivo. In other embodiments, the engineered cells are transfected or transduced ex vivo.

The methods can further comprise administering one or more chemotherapeutic agent. In certain embodiments, the chemotherapeutic agent is a lymphodepleting (preconditioning) conditioning) chemotherapeutic. Beneficial preconditioning treatment regimens, along with correlative beneficial biomarkers are described in U.S. Provisional Patent Applications 62/262,143 and 62/167,750 which are hereby incorporated by reference in their entirety herein. These describe, e.g., methods of conditioning a patient in need of a T cell therapy comprising administering to the patient specified beneficial doses of cyclophosphamide (between 200 mg/m²/day and 2000 mg/m²/day) and specified doses of fludarabine (between 20 mg/m²/day and 900 mg/m²/day). A preferred dose regimen involves treating a patient comprising administering daily to the patient about 500 mg/m²/day of cyclophosphamide and about 60 mg/m²/day of fludarabine for three days prior to administration of a therapeutically effective amount of engineered T cells to the patient.

In other embodiments, the antigen binding molecule, transduced (or otherwise engineered) cells (such as CARs or TCRs), and the chemotherapeutic agent are administered each in an amount effective to treat the disease or condition in the subject.

In certain embodiments, compositions comprising CAR-expressing immune effector cells disclosed herein may be administered in conjunction with any number of chemotherapeutic agents. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, my cophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-tri chlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hy droxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovie), and Prednisone.

In some embodiments, the chemotherapeutic agent is administered at the same time or within one week after the administration of the engineered cell or nucleic acid. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the engineered cell or nucleic acid. In other embodiments, the chemotherapeutic agent is administered at least 1 month before administering the cell or nucleic acid. In some embodiments, the methods further comprise administering two or more chemotherapeutic agents.

A variety of additional therapeutic agents may be used in conjunction with the compositions described herein. For example, potentially useful additional therapeutic agents include PD-1 inhibitors such as nivolumab (Opdivo®), pembrolizumab (Keytruda®), pembrolizumab, pidilizumab, and atezolizumab.

Additional therapeutic agents suitable for use in combination with the invention include, but are not limited to, ibrutinib (Imbruvice), ofatumumab (Arzerra®), rituximab (Rituxan®), bevacizumab (Avastin®), trastuzumab (Herceptin®), trastuzumab emtansine (KADCYLA®), imatinib (Gleevec®), cetuximab (Erbitux®), panitumumab (Vectibix®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept,adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In additional embodiments, the composition comprising CAR-containing immune can be administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

In certain embodiments, the compositions described herein are administered in conjunction with a cytokine. “Cytokine” as used herein is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1 IL-lalpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In some aspects, the invention comprises an antigen binding molecule that binds to FLT3 with a K_{d} that is smaller than 100 pM. In some embodiments, the antigen binding molecule binds with a K_{d} that is smaller than 10 pM. In other embodiments, the antigen binding molecule binds with a K_{d} that is less than 5 pM.

### Methods of Making

A variety of known techniques can be utilized in making the polynucleotides, polypeptides, vectors, antigen binding molecules, immune cells, compositions, and the like according to the invention.

Prior to the in vitro manipulation or genetic modification of the immune cells described herein, the cells may be obtained from a subject. In some embodiments, the immune cells comprise T cells. T cells can be obtained from a number of sources, including peripheral blood mononuclear cells (PBMCs), bone marrow, lymph nodes tissue, cord blood, thymus tissue, tissue from a site of infection, ascites, pleural effusion, spleen tissue, and tumors. In certain embodiments, T cells can be obtained from a unit of blood collected from the subject using any number of techniques known to the skilled person, such as FICOLL™ separation. Cells may preferably be obtained from the circulating blood of an individual by apheresis. The apheresis product typically contains lymphocytes, including T cells, monocytes, granulocytes, B cells, other nucleated white blood cells, red blood cells, and platelets. In certain embodiments, the cells collected by apheresis may be washed to remove the plasma fraction, and placed in an appropriate buffer or media for subsequent processing. The cells may be washed with PBS. As will be appreciated, a washing step may be used, such as by using a semiautomated flowthrough centrifuge—for example, the Cobe™ 2991 cell processor, the Baxter CytoMate™, or the like. After washing, the cells may be resuspended in a variety of biocompatible buffers, or other saline solution with or without buffer. In certain embodiments, the undesired components of the apheresis sample may be removed.

In certain embodiments, T cells are isolated from PBMCs by lysing the red blood cells and depleting the monocytes, for example, using centrifugation through a PERCOLL™ gradient. A specific subpopulation of T cells, such as CD28⁺, CD4⁺, CD8⁺, CD45RA⁺, and CD45RO⁺ T cells can be further isolated by positive or negative selection techniques known in the art. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. Flow cytometry and cell sorting may also be used to isolate cell populations of interest for use in the present invention.

PBMCs may be used directly for genetic modification with the immune cells (such as CARs or TCRs) using methods as described herein. In certain embodiments, after isolating the PBMCs, T lymphocytes can be further isolated and both cytotoxic and helper T lymphocytes can be sorted into naive, memory, and effector T cell subpopulations either before or after genetic modification and/or expansion.

In some embodiments, CD8⁺ cells are further sorted into naive, central memory, and effector cells by identifying cell surface antigens that are associated with each of these types of CD8⁺ cells. In some embodiments, the expression of phenotypic markers of central memory T cells include CD45RO, CD62L, CCR7, CD28, CD3, and CD127 and are negative for granzyme B. In some embodiments, central memory T cells are CD45RO⁺, CD62L⁺, CD8⁺ T cells. In some embodiments, effector T cells are negative for CD62L, CCR7, CD28, and CD127, and positive for granzyme B and perforin. In certain embodiments, CD4⁺ T cells are further sorted into subpopulations. For example, CD4⁺ T helper cells can be sorted into naive, central memory, and effector cells by identifying cell populations that have cell surface antigens.

The immune cells, such as T cells, can be genetically modified following isolation using known methods, or the immune cells can be activated and expanded (or differentiated in the case of progenitors) in vitro prior to being genetically modified. In another embodiment, the immune cells, such as T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, for example, in U.S. Pat. Nos. 6,905,874; 6,867,041; 6,797,514; and PCT WO2012/079000, the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is The Dynabeads® system, a CD3/CD28 activator/stimulator system for physiological activation of human T cells.

In other embodiments, the T cells may be activated and stimulated to proliferate with feeder cells and appropriate antibodies and cytokines using methods such as those described in U.S. Pat. Nos. 6,040,177; 5,827,642; and WO2012129514, the contents of which are hereby incorporated by reference in their entirety.

Certain methods for making the constructs and engineered immune cells of the invention are described in PCT application PCT/US15/14520, the contents of which are hereby incorporated by reference in their entirety. Additional methods of making the constructs and cells can be found in U.S. provisional patent application no. 62/244036 the contents of which are hereby incorporated by reference in their entirety.

It will be appreciated that PBMCs can further include other cytotoxic lymphocytes such as NK cells or NKT cells. An expression vector carrying the coding sequence of a chimeric receptor as disclosed herein can be introduced into a population of human donor T cells, NK cells or NKT cells. Successfully transduced T cells that carry the expression vector can be sorted using flow cytometry to isolate CD3 positive T cells and then further propagated to increase the number of these CAR expressing T cells in addition to cell activation using anti-CD3 antibodies and IL-2 or other methods known in the art as described elsewhere herein. Standard procedures are used for cryopreservation of T cells expressing the CAR for storage and/or preparation for use in a human subject. In one embodiment, the in vitro transduction, culture and/or expansion of T cells are performed in the absence of non-human animal derived products such as fetal calf serum and fetal bovine serum.

For cloning of polynucleotides, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vector provided herein. The host cells containing the vector may be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example,

Enterobactehaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art. In a further embodiment, a mixture of different expression vectors can be used in genetically modifying a donor population of immune effector cells wherein each vector encodes a different CAR as disclosed herein. The resulting transduced immune effector cells form a mixed population of engineered cells, with a proportion of the engineered cells expressing more than one different CARs.

In one embodiment, the invention provides a method of storing genetically engineered cells expressing CARs or TCRs which target a FLT3 protein. This involves cryopreserving the immune cells such that the cells remain viable upon thawing. A fraction of the immune cells expressing the CARs can be cryopreserved by methods known in the art to provide a permanent source of such cells for the future treatment of patients afflicted with a malignancy. When needed, the cryopreserved transformed immune cells can be thawed, grown and expanded for more such cells.

As used herein, “cryopreserve” refers to the preservation of cells by cooling to sub-zero temperatures, such as (typically) 77 Kelvin or −196° C. (the boiling point of liquid nitrogen). Cryoprotective agents are often used at sub-zero temperatures to prevent the cells being preserved from damage due to freezing at low temperatures or warming to room temperature. Cryopreservative agents and optimal cooling rates can protect against cell injury. Cryoprotective agents which can be used in accordance with the invention include but are not limited to: dimethyl sulfoxide (DMSO) (Lovelock & Bishop, Nature (1959); 183: 1394-1395; Ashwood-Smith, Nature (1961); 190: 1204-1205), glycerol, polyvinylpyrrolidine (Rinfret, Ann. N.Y. Acad. Sci. (1960); 85: 576), and polyethylene glycol (Sloviter & Ravdin, Nature (1962); 196: 48). The preferred cooling rate is 1°-3° C./minute.

The term, “substantially pure,” is used to indicate that a given component is present at a high level. The component is desirably the predominant component present in a composition. Preferably it is present at a level of more than 30%, of more than 50%, of more than 75%, of more than 90%, or even of more than 95%, said level being determined on a dry weight/dry weight basis with respect to the total composition under consideration. At very high levels (e.g. at levels of more than 90%, of more than 95% or of more than 99%) the component can be regarded as being in “pure form.” Biologically active substances of the present invention (including polypeptides, nucleic acid molecules, antigen binding molecules, moieties) can be provided in a form that is substantially free of one or more contaminants with which the substance might otherwise be associated. When a composition is substantially free of a given contaminant, the contaminant will be at a low level (e.g., at a level of less than 10%, less than 5%, or less than 1% on the dry weight/dry weight basis set out above).

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer's lactate can be utilized. The infusion medium can be supplemented with human serum albumin.

Desired treatment amounts of cells in the composition is generally at least 2 cells (for example, at least 1 CD8⁺ central memory T cell and at least 1 CD4⁺ helper T cell subset) or is more typically greater than 10² cells, and up to 10⁶, up to and including 10⁸ or 10⁹ cells and can be more than 10¹⁰ cells. The number of cells will depend upon the desired use for which the composition is intended, and the type of cells included therein. The density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present invention, particularly since all the infused cells will be redirected to a particular target antigen (FLT3), lower numbers of cells, in the range of 10⁶/kilogram (10⁶-10¹¹ per patient) may be administered. CAR treatments may be administered multiple times at dosages within these ranges. The cells may be autologous, allogeneic, or heterologous to the patient undergoing therapy.

The CAR expressing cell populations of the present invention may be administered either alone, or as a pharmaceutical composition in combination with diluents and/or with other components such as IL-2 or other cytokines or cell populations. Pharmaceutical compositions of the present invention may comprise a CAR or TCR expressing cell population, such as T cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

The pharmaceutical compositions (solutions, suspensions or the like), may include one or more of the following: sterile diluents such as water for injection, saline solution, preferably physiological saline, Ringer's solution, isotonic sodium chloride, fixed oils such as synthetic mono- or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

It will be appreciated that adverse events may be minimized by transducing the immune cells (containing one or more CARs or TCRs) with a suicide gene. It may also be desired to incorporate an inducible “on” or “accelerator” switch into the immune cells. Suitable techniques include use of inducible caspase-9 (U.S. Appl. 2011/0286980) or a thymidine kinase, before, after or at the same time, as the cells are transduced with the CAR construct of the present invention. Additional methods for introducing suicide genes and/or “on” switches include TALENS, zinc fingers, RNAi, siRNA, shRNA, antisense technology, and other techniques known in the art.

It will be understood that descriptions herein are exemplary and explanatory only and are not restrictive of the invention as claimed. In this application, the use of the singular includes the plural unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. As utilized in accordance with the present disclosure, the following terms, unless otherwise indicated, shall be understood to have the following meanings:

In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The term “FLT3 activity” includes any biological effect of FLT3. In certain embodiments, FLT3 activity includes the ability of FLT3 to interact or bind to a substrate or receptor.

The term “polynucleotide”, “nucleotide”, or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphoro-diselenoate, phosphoro-anilothioate, phoshoraniladate and phosphoroamidate.

The term “oligonucleotide” refers to a polynucleotide comprising 200 or fewer nucleotides. Oligonucleotides can be single stranded or double stranded, e.g., for use in the construction of a mutant gene. Oligonucleotides can be sense or antisense oligonucleotides. An oligonucleotide can include a label, including a radiolabel, a fluorescent label, a hapten or an antigenic label, for detection assays. Oligonucleotides can be used, for example, as PCR primers, cloning primers or hybridization probes.

The term “control sequence” refers to a polynucleotide sequence that can affect the expression and processing of coding sequences to which it is ligated. The nature of such control sequences can depend upon the host organism. In particular embodiments, control sequences for prokaryotes can include a promoter, a ribosomal binding site, and a transcription termination sequence. For example, control sequences for eukaryotes can include promoters comprising one or a plurality of recognition sites for transcription factors, transcription enhancer sequences, and transcription termination sequence. “Control sequences” can include leader sequences (signal peptides) and/or fusion partner sequences.

As used herein, “operably linked” means that the components to which the term is applied are in a relationship that allows them to carry out their inherent functions under suitable conditions.

The term “vector” means any molecule or entity (e.g., nucleic acid, plasmid, bacteriophage or virus) used to transfer protein coding information into a host cell. The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct can include, but is not limited to, sequences that affect or control transcription, translation, and, if introns are present, affect RNA splicing of a coding region operably linked thereto.

The term “host cell” refers to a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell, or can be maintained transiently as an episomal element without being replicated, or can replicate independently as a plasmid. A cell is considered to have been “stably transformed” when the transforming DNA is replicated with the division of the cell.

The term “transfection” refers to the uptake of foreign or exogenous DNA by a cell. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197.

The term “transduction” refers to the process whereby foreign DNA is introduced into a cell via viral vector. See Jones et al., (1998). Genetics: principles and analysis. Boston: Jones & Bartlett Publ.

The terms “polypeptide” or “protein” refer to a macromolecule having the amino acid sequence of a protein, including deletions from, additions to, and/or substitutions of one or more amino acids of the native sequence. The terms “polypeptide” and “protein” specifically encompass FLT3 antigen binding molecules, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. Useful polypeptide fragments include immunologically functional fragments of antigen binding molecules. Useful fragments include but are not limited to one or more CDR regions, variable domains of a heavy and/or light chain, a portion of other portions of an antibody chain, and the like.

The term “isolated” means (i) free of at least some other proteins with which it would normally be found, (ii) is essentially free of other proteins from the same source, e.g., from the same species, (iii) separated from at least about 50 percent of polynucleotides, lipids, carbohydrates, or other materials with which it is associated in nature, (iv) operably associated (by covalent or noncovalent interaction) with a polypeptide with which it is not associated in nature, or (v) does not occur in nature.

A “variant” of a polypeptide (e.g., an antigen binding molecule, or an antibody) comprises an amino acid sequence wherein one or more amino acid residues are inserted into, deleted from and/or substituted into the amino acid sequence relative to another polypeptide sequence. Variants include fusion proteins.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”).

To calculate percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

As used herein, the twenty conventional (e.g., naturally occurring) amino acids and their abbreviations follow conventional usage. See Immunology - A Synthesis (2nd Edition, Golub and Gren, Eds., Sinauer Assoc., Sunderland, Mass. (1991)), which is incorporated herein by reference for any purpose. Stereoisomers (e.g., D-amino acids) of the twenty conventional amino acids, unnatural amino acids such as alpha-, alpha-disubstituted amino acids, N-alkyl amino acids, lactic acid, and other unconventional amino acids can also be suitable components for polypeptides of the present invention. Examples of unconventional amino acids include: 4-hydroxyproline, .gamma.-carboxyglutamate, epsilon-N,N,N-trimethyllysine, e-N-acetyllysine, O-phosphoserine, N-acetylserine, N-formylmethionine, 3-methylhistidine, 5-hydroxylysine, .sigma.-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Conservative amino acid substitutions can encompass non-naturally occurring amino acid residues, which are typically incorporated by chemical peptide synthesis rather than by synthesis in biological systems. These include peptidomimetics and other reversed or inverted forms of amino acid moieties. Naturally occurring residues can be divided into classes based on common side chain properties:
a) hydrophobic: norleucine, Met, Ala, Val, Leu, Ile;
b) neutral hydrophilic: Cys, Ser, Thr, Asn, Gln;
c) acidic: Asp, Glu;
d) basic: His, Lys, Arg;
e) residues that influence chain orientation: Gly, Pro; and
f) aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the antigen binding molecule, the costimulatory or activating domains of the engineered T cell, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). See Kyte et al., J. Mol. Biol., 157:105-131 (1982). It is known that certain amino acids can be substituted for other amino acids having a similar hydropathic index or score and still retain a similar biological activity. It is also understood in the art that the substitution of like amino acids can be made effectively on the basis of hydrophilicity, particularly where the biologically functional protein or peptide thereby created is intended for use in immunological embodiments, as in the present case. Exemplary amino acid substitutions are set forth in Table 2.

TABLE 2

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, | Leu |
| | Norleucine | |
| Leu | Norleucine, Ile, Val, Met, Ala, | Ile |
| | Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, | Leu |
| | Tyr | |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen binding molecule can have a greater circulating half-life than an antigen binding molecule that is not chemically modified. In some embodiments, a derivative antigen binding molecule is covalently modified to include one or more water soluble polymer attachments, including, but not limited to, polyethylene glycol, polyoxyethylene glycol, or polypropylene glycol.

Peptide analogs are commonly used in the pharmaceutical industry as non-peptide drugs with properties analogous to those of the template peptide. These types of non-peptide compound are termed “peptide mimetics” or “peptidomimetics.” Fauchere, J., Adv. Drug Res., 15:29 (1986); Veber & Freidinger, TINS, p.392 (1985); and Evans et al., J. Med. Chem., 30:1229 (1987), which are incorporated herein by reference for any purpose.

The term “therapeutically effective amount” refers to the amount of a FLT3 antigen binding molecule determined to produce a therapeutic response in a mammal. Such therapeutically effective amounts are readily ascertained by one of ordinary skill in the art.

The terms “patient” and “subject” are used interchangeably and include human and non-human animal subjects as well as those with formally diagnosed disorders, those without formally recognized disorders, those receiving medical attention, those at risk of developing the disorders, etc.

The term “treat” and “treatment” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. The term “prevent” does not require the 100% elimination of the possibility of an event. Rather, it denotes that the likelihood of the occurrence of the event has been reduced in the presence of the compound or method.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

The following sequences will further exemplify the invention.

CD28T DNA Extracellular, transmembrane. intracellular
(SEQ ID NO: 1)
CTTGATAATGAAAAGTC
AAACGGAACAATCATTCACGTGAAGGGCAAGCACCTCTGTCCGTCACC
CTTGTTCCCTGGTCCATCCAAGCCATTCTGGGTGTTGGTCGTAGTGGGT
GGAGTCCTCGCTTGTTACTCTCTGCTCGTCACCGTGGCTTTTATAATCTT
CTGGGTTAGATCCAAAAGAAGCCGCCTGCTCCATAGCGATTACATGAA
TATGACTCCACGCCGCCCTGGCCCCACAAGGAAACACTACCAGCCTTA
CGCACCACCTAGAGATTTCGCTGCCTATCGGAGC
CD28T Extracellular, transmembrane, intracellular AA:
(SEQ ID NO: 2)
LDNEKSNGTIIHVKGKHLCPSPLFPGPSKPFWVLVVVGGV
LACYSLLVTVAFIIFWVRSKRSRLLHSDYMNMTPRRPGPT
RKHYQPYAPP RDFAAYRS
CD28T DNA-Extracellular
(SEQ ID NO: 3)
CTTGATAATGAAAAGTCAAACGGAACAATCATTCACGTGAAGGGCAA
GCACCTCTGTCCGTCACCCTTGTTCCCTGGTCCATCCAAGCCA
CD28T AA-Extracellular
(SEQ ID NO: 4)
LDNEKSNGTI IHVKGKHLCP SPLFPGPSKP
CD28 DNA Transmembrane Domain
(SEQ ID NO: 5)
TTCTGGGTGTTGGTCGTAGTGGGTGGAGTCCTCGCTTGTTACTCTCTGC
TCGTCACCGTGGCTTTTATAATCTTCTGGGTT
CD28 AA Transmembrane Domain:
(SEQ ID NO: 6)
FWVLVVVGGV LACYSLLVTV AFIIFWV
CD28 DNA Intracellular Domain:
(SEQ ID NO: 7)
AGATCCAAAAGAAGCCGCCTGCTCCATAGCGATTACATGAATATGACT
CCACGCCGCCCTGGCCCCACAAGGAAACACTACCAGCCTTACGCACCA
CCTAGAGATTTCGCTGCCTATCGGAGC
CD28 AA Intracellular Domain
(SEQ ID NO: 8)
RSKRSRLLHSDYMNMTPRRPGPTRKHYQPYAPPRDFAAYRS
CD3 zeta DNA
(SEQ ID NO: 9)
AGGGTGAAGTTTTCCAGATCTGCAGATGCACCAGCGTATCAGCAGGGC
CAGAACCAACTGTATAACGAGCTCAACCTGGGACGCAGGGAAGAGTA
TGACGTTTTGGACAAGCGCAGAGGACGGGACCCTGAGATGGGTGGCA
AACCAAGACGAAAAAACCCCCAGGAGGGTCTCTATAATGAGCTGCAG
AAGGATAAGATGGCTGAAGCCTATTCTGAAATAGGCATGAAAGGAGA
GCGGAGAAGGGGAAAAGGGCACGACGGTTTGTACCAGGGACTCAGCA
CTGCTACGAAGGATACTTATGACGCTCTCCACATGCAAGCCCTGCCAC
CTAGG
CD3 zeta AA
(SEQ ID NO: 10)
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTA
TKDTYDALHMQALPPR
CD28 DNA
(SEQ ID NO: 11)
ATTGAGGTGATGTATCCACCGCCTTACCTGGATAACGAAAAGAGTAAC
GGTACCATCATTCACGTGAAAGGTAAACACCTGTGTCCTTCTCCCCTCT
TCCCCGGGCCATCAAAGCCC
CD28 AA
(SEQ ID NO: 12)
IEVMYPPPYL DNEKSNGTII HVKGKHLCPS PLFPGPSKP
CD8 DNA extracellular & transmembrane domain
(SEQ ID NO: 13)
GCTGCAGCATTGAGCAACTCAATAATGTATTTTAGTCACTTTGTACCAG
TGTTCTTGCCGGCTAAGCCTACTACCACACCCGCTCCACGGCCACCTAC
CCCAGCTCCTACCATCGCTTCACAGCCTCTGTCCCTGCGCCCAGAGGCT
TGCCGACCGGCCGCAGGGGGCGCTGTTCATACCAGAGGACTGGATTTC
GCCTGCGATATCTATATCTGGGCACCCCTGGCCGGAACCTGCGGCGTA
CTCCTGCTGTCCCTGGTCATCACGCTCTATTGTAATCACAGGAAC
CD 8 AA extracellular & transmembrane Domain
(SEQ ID NO: 14)
AAALSNSIMYFSHFVPVFLPAKPTTTPAPRPPTPAPTIASQPLSLRPEACRPA
AGGAVHTRGLDFACDIYIWAPLAGTCGVLLLSLVITLYCNHRN
Clone 10E3 HC DNA
(SEQ ID NO: 15)
CAGGTCACCTTGAAGGAGTCTGGTCCTGTGCTGGTGAAACCCACAGAG
ACCCTCACGCTGACCTGCACCGTCTCTGGGTTCTCACTCATCAATGCTA
GAATGGGTGTGAGCTGGATCCGTCAGCCCCCAGGGAAGGCCCTGGAGT
GGCTTGCACACATTTTTTCGAATGCCGAAAAATCGTACAGGACATCTC
TGAAGAGCAGGCTCACCATCTCCAAGGACACCTCCAAAAGCCAGGTG
GTCCTTACCATGACCAACATGGACCCTGTGGACACAGCCACATATTAC
TGTGCACGGATACCAGGCTACGGTGGTAACGGGGACTACCACTACTAC
GGTATGGACGTCTGGGGCCAAGGGACCACGGTCACCGTCTCCTCA
Clone 10E3 HC AA-CDRs Underlined
(SEQ ID NO: 16)
QVTLKESGPVLVKPTETLTLTCTVSGFSLINARMGVSWIRQPPGKALEWL
AHIFSNAEKSYRTSLKSRLTISKDTSKSQVVLTMTNMDPVDTATYYCARIP
GYGGNGDYHYYGMDVWGQGTTVTVSS
Clone 10E3 HC AA CDR1:
(SEQ ID NO: 17)
NARMGVS
Clone 10E3 HC AA CDR2:
(SEQ ID NO: 18)
HIFSNAEKSYRTSLKS
Clone 10E3 HC AA CDR3:
(SEQ ID NO: 19)
IPGYGGNGDYHYYGMDV
Clone 10E3 LC DNA
(SEQ ID NO: 20)
GACATCCAGATGACCCAGTCTCCATCCTCCCTGTCTGCATCTCTAGGAG
ACAGAGTCACCATCACTTGCCGGGCAAGTCAGGGCATTAGAAATGATT
TAGGCTGGTATCAGCAGAAACCAGGGAAAGCCCCTAAGCGCCTGATCT
ATGCTTCATCCACTTTGCAAAGTGGGGTCCCATCAAGGTTCAGCGGCA
GTGGATCTGGGACAGAGTTCACTCTCACAATCAGCAGCCTGCAGCCTG
AAGATTTTGCAACTTATTACTGTCTACAGCATAATAATTTCCCGTGGAC
GTTCGGTCAGGGAACGAAGGTGGAAATCAAACGA
Clone 10E3 LC AA (CDRs Underlined)
(SEQ ID NO: 21)
DIQMTQSPSSLSASLGDRVTITCRASQGIRNDLGWYQQKPGKAPKRLIYAS
STLQSGVPSRFSGSGSGTEFTLTISSLQPEDFATYYCLQHNNFPWTFGQGT
KVEIKR
Clone 10E3 LC CDR1 AA:
(SEQ ID NO: 22)
RASQGIRNDLG
Clone 10E3 LC CDR2 AA:
(SEQ ID NO: 23)
ASSTLQS
Clone 10E3 LC CDR3 AA:
(SEQ ID NO: 24)
LQHNNFPWT
Clone 2E7 HC DNA
(SEQ ID NO: 25)
CAGGTCACCTTGAAGGAGTCTGGTCCTGTGCTGGTGAAACCCACAGAGACCCTCA
CGCTGACCTGCACCGTCTCTGGGTTCTCACTCAGGAATGCTAGAATGGGTGTAAG
CTGGATCCGTCAGCCTCCCGGGAAGGCCCTGGAGTGGCTTGCACACATTTTTTCG
AATGACGAAAAAACCTACAGCACATCTCTGAAGAGCAGGCTCACCATCTCCAGG
GACACCTCCAAAGGCCAGGTGGTCCTTACCATGACCAAGATGGACCCTGTGGAC
ACAGCCACATATTACTGTGCACGGATACCCTACTATGGTTCGGGGAGTCATAACT
ACGGTATGGACGTCTGGGGCCAAGGGACCACGGTCACCGTCTCCTCA
2E7 HC AA (CDRs underlined)
(SEQ ID NO: 26)
QVTLKESGPVLVKPTETLTLTCTVSGFSLRNARMGVSWIRQPPGKALEWLAHIFSND
EKTYSTSLKSRLTISRDTSKGQVVLTMTKMDPVDTATYYCARIPYYGSGSHNYGMD
VWGQGTTVTVSS
2E7 HC AA CDR1:
(SEQ ID NO: 17)
NARMGVS
2E7 HC AA CDR2:
(SEQ ID NO: 26)
HIFSNDEKTYSTSLKS
2E7 HC AA CDR3:
(SEQ ID NO 27)
IPYYGSGSHNYGMDV
2E7 LC DNA
(SEQ ID NO: 28)
GACATCCAGATGACCCAGTCTCCATCCTCCCTGTCTGCATCTGTAGGAGACAGAG
TCACCATCACTTGCCGGGCAAGTCAGGACATTAGAAATGATTTCGGCTGGTATCA
ACAGAAACCAGGGAAAGCCCCTCAGCGCCTGCTCTATGCTGCATCCACTTTGCAA
AGTGGGGTCCCATCAAGGTTCAGCGGCAGTGGATCTGGGACAGAATTCACTCTC
ACAATCAGCAGCCTGCAGCCTGAAGATTTTGCAACTTATTACTGTCTACAGTATA
ATACTTACCCGTGGACGTTCGGTCAGGGAACGAAGGTGGAAATCAAACGA
Clone 2E7 LC AA (CDRs underlined)
(SEQ ID NO: 29)
DIQMTQSPSSLSASVGDRVTITCRASQDIRNDFGWYQQKPGKAPQRLLYAASTLQSG
VPSRFSGSGSGTEFTLTISSLQPEDFATYYCLQYNTYPWTFGQGTKVEIKR
Clone 2E7 LC AA CDR1:
(SEQ ID NO: 30)
RASQDIRNDFG
Clone 2E7 LC AA CDR2:
(SEQ ID NO: 31)
AASTLQS
Clone 2E7 LHC AA CDR3:
(SEQ ID NO: 32)
LQYNTYPWT
Clone 8B5 HC DNA
(SEQ ID NO: 33)
CAGATACAACTGGTGGAGTCTGGGGGAGGCGTGGTCCAGCCTGGGAGGTCCCTG
AGACTCTCCTGTGTAGCGTCTGGATTCACCTTCAAGAACTATGGCATGCACTGGG
TCCGCCAGGCTCCAGGCAAGGGGCTGGAGTGGGTGGCAGTTATTTGGTATGATG
GAAGTAATGAATACTATGGAGACCCCGTGAAGGGCCGATTCACCATCTCCAGAG
ACAACTCCAAGAACATGTTGTATCTGCAAATGAACAGCCTGAGAGCCGATGACA
CGGCTGTGTATTACTGTGCGAGGTCGGGAATAGCAGTGGCTGGGGCCTTTGACTA
CTGGGGCCAGGGAACCCTGGTCACCGTCTCCTCA
Clone 8B5 HC AA (CDRs underlined)
(SEQ ID NO: 34)
QIQLVESGGGVVQPGRSLRLSCVASGFTFKNYGMHWVRQAPGKGLEWVAVIWYDG
SNEYYGDPVKGRFTISRDNSKNMLYLQMNSLRADDTAVYYCARSGIAVAGAFDYW
GQGTLVTVSS
Clone 8B5 HC AA CDR1:
(SEQ ID NO: 34)
NYGMH
Clone 8B5 HC AA CDR2:
(SEQ ID NO: 35)
VIWYDGSNEYYGDPVKG
Clone 8B5 HC AA CDR3:
(SEQ ID NO: 36)
SGIAVAGAFDY
Clone 8B5 LC DNA
(SEQ ID NO: 37)
GAAATTGTGTTGACGCAGTCTCCAGACACCCTGTCTTTGTCTCCAGGGGAAAAAG
CCACCCTCTCCTGCAGGGCCAGTCAGAGTGTTAGCAGCAGCTTCTTGGCCTGGTA
CCAGCAGAAACCTGGACAGGCTCCCAGTCTCCTCATCTATGTTGCATCCAGAAGG
GCCGCTGGCATCCCTGACAGGTTCAGTGGCAGTGGGTCTGGGACAGACTTCACTC
TCACCATCAGCAGACTGGAGCCTGAAGATTTTGGAATGTTTTACTGTCAACACTA
TGGTAGGACACCATTCACTTTCGGCCCTGGGACCAAAGTGGATATCAAACGA
Clone 8B5 LC AA (CDRs underlined)
(SEQ ID NO: 41)
EIVLTQSPDTLSLSPGEKATLSCRASQSVSSSFLAWYQQKPGQAPSLLIYVASRRAAGI
PDRFSGSGSGTDFTLTISRLEPEDFGMFYCQHYGRTPFTFGPGTKVDIKR
Clone 8B5 LC AA CDR1:
(SEQ ID NO: 38)
RASQSVSSSFLA
Clone 8B5 LC AA CDR2:
(SEQ ID NO: 39)
VASRRAA
Clone 8B5 LC AA CDR3:
(SEQ ID NO: 40)
QHYGRTPFT
Clone 4E9 HC DNA
(SEQ ID NO: 41)
CAGGTGCAGCTGGTGCAGTCTGGGGCTGAGGTGAAGAAGCCTGGGGCCTCAGTG
AAGGTCTCCTGCAAGGCTTCTGGATACACCTTCACCGGCTACTATATACACTGGG
TGCGACAGGCCCCTGAACAAGGGCTTGAGTGGATGGGATGGATCAACCCTAACA
GTGGTGGCACAAACTATGCACAGAAGTTTCAGGGCAGGGTCACCATGGCCAGGG
ACACGTCCATCAGCACAGTTTACATGGACCTGAGCAGGCTGAGATCTGACGACA
CGGCCGTGTATTACTGTGCGAGAATACGCGGTGGTAACTCGGTCTTTGACTACTG
GGGCCAGGGAACCCTGGTCACCGTCTCCTCA
Clone 4E9 HC AA (CDRs underlined)
(SEQ ID NO: 42)
QVQLVQSGAEVKKPGASVKVSCKASGYTFTGYYIHWVRQAPEQGLEWMGWINPNS
GGTNYAQKFQGRVTMARDTSISTVYMDLSRLRSDDTAVYYCARIRGGNSVFDYWG
QGTLVTVSS
Clone 4E9 HC AA CDR1:
(SEQ ID NO: 43)
GYYIH
Clone 4E9 HC AA CDR2:
(SEQ ID NO: 44)
WINPNSGGTNYAQKFQG
Clone 4E9 HC AA CDR3:
(SEQ ID NO: 45)
IRGGNSVFDY
Clone 4E9 LC DNA
(SEQ ID NO: 46)
GACATCGTGATGACCCAGTCTCCAGACTCCCTGGCTGTGTCTCTGGGCGAGAGGG
CCACCATCAACTGCAAGTCCACCCAGAGTATTTTATACACCTCCAACAATAAGAA
CTTCTTAGCTTGGTACCAGCAGAAACCAGGGCAGCCTCCTAAACTGCTCATTTCC
TGGGCATCTATCCGGGAATCCGGGGTCCCTGACCGATTCAGTGGCAGCGGGTCTG
GGACAGATTTCGCTCTCACCATCAGCAGCCTGCAGGCTGAAGATGTGGCAGTTTA
TTACTGTCAACAATATTTTAGTACTATGTTCAGTTTTGGCCAGGGGACCAAGCTG
GAGATCAAACGA
Clone 4E9 LC AA (CDRs underlined)
(SEQ ID NO: 47)
DIVMTQSPDSLAVSLGERATINCKSTQSILYTSNNKNFLAWYQQKPGQPPKLLISWAS
IRESGVPDRFSGSGSGTDFALTISSLQAEDVAVYYCQQYFSTMFSFGQGTKLEIKR
Clone 4E9 LC AA CDR1:
(SEQ ID NO: 48)
KSTQSILYTSNNKNFLA
Clone 4E9 LC AA CDR2:
(SEQ ID NO: 49)
WASIRES
Clone 4E9 LC AA CDR3:
(SEQ ID NO: 50)
QQYFSTMFS
Clone 11F11 HC DNA
(SEQ ID NO: 51)
CAGGTGCAGCTGCAGGAGTCGGGCCCAGGACTGGTGAAGCCTTCACAGACCCTG
TCCCTCACCTGCACTGTCTCTGGTGGCTCCATCAGTAGTGGTGCATACTACTGGA
CTTGGATCCGCCAGCACCCAGGGAAGGGCCTGGAGTGGATTGGGTACATCCATT
ACAGTGGGAGCACCTACTCCAACCCGTCCCTCAAGAGTCGAATTACCATATCGTT
AGACACGTCTAAGAACCAGTTCTCCCTGAAGCTGAACTCTGTGACTGCCGCGGAC
ACGGCCGTGTATTACTGTGCGAGACAAGAGGACTACGGTGGTTTGTTTGACTACT
GGGGCCAGGGAACCCTGGTCACCGTTTCCTCA
Clone 11F11 HC AA (CDRs underlined)
(SEQ ID NO: 52)
QVQLQESGPGLVKPSQTLSLTCTVSGGSISSGAYYWTWIRQHPGKGLEWIGYIHYSG
STYSNPSLKSRITISLDTSKNQFSLKLNSVTAADTAVYYCARQEDYGGLFDYWGQGT
LVTVSS
Clone 11F11 HC AA CDR1:
(SEQ ID NO: 53)
SGAYYWT
Clone 11F1 HC AA CDR2:
(SEQ ID NO: 54)
YIHYSGSTYSNPSLKS
Clone 11F1 HC AA CDR3:
(SEQ ID NO: 55)
QEDYGGLFDY
Clone 11F11 LC DNA
(SEQ ID NO: 56)
GAAATAGTGATGACGCAGTCTCCAGCCACCCTGTCTGTGTCTCCAGGGGAAAGA
ATCACCCTCTCCTGCAGGGCCAGTCAGAGTGTTACCACCGACTTAGCCTGGTACC
AGCAGATGCCTGGACAGGCTCCCCGGCTCCTCATCTATGATGCTTCCACCAGGGC
CACTGGTTTCCCAGCCAGATTCAGTGGCAGTGGGTCTGGGACAGACTTCACGCTC
ACCATCAGCAGCCTGCAGGCTGAAGATTTTGCAGTTTATTACTGTCAACATTATA
AAACCTGGCCTCTCACTTTCGGCGGAGGGACTAAGGTGGAGATCAAACGA
Clone 11F11 LC AA (CDRs underlined)
(SEQ ID NO: 57)
EIVMTQSPATLSVSPGERITLSCRASQSVTTDLAWYQQMPGQAPRLLIYDASTRATGF
PARFSGSGSGTDFTLTISSLQAEDFAVYYCQHYKTWPLTFGGGTKVEIKR
Clone 11F11 LC AA CDR1:
(SEQ ID NO: 58)
RASQSVTTDLA
Clone 11F1 LC AA CDR2:
(SEQ ID NO: 59)
DASTRAT
Clone 11F1 LC AACDR3:
(SEQ ID NO: 60)
QHYKTWPLT
Construct 10E3 CD28 DNA (signal sequence in bold)
(SEQ ID NO: 61)
ATGGCACTCCCCGTAACTGCTCTGCTGCTGCCGTTGGCATTGCTCCTGCACG
CCGCACGCCCGCAGGTGACCCTCAAAGAGTCTGGACCCGTGCTCGTAAAACCTA
CGGAGACCCTGACACTCACCTGCACAGTCTCCGGCTTCAGCCTCATCAATGCCAG
GATGGGAGTTTCCTGGATCAGGCAACCGCCCGGAAAGGCCCTGGAATGGCTCGC
ACATATTTTCAGTAACGCTGAAAAAAGCTATCGGACTTCTCTGAAAAGTCGGCTC
ACGATTAGTAAGGACACATCCAAGAGCCAAGTGGTGCTTACGATGACTAACATG
GACCCTGTGGATACTGCAACCTATTACTGTGCTCGAATCCCTGGTTATGGCGGAA
ATGGGGACTACCACTACTACGGTATGGATGTCTGGGGCCAAGGGACCACGGTTA
CTGTTTCAAGCGGAGGGGGAGGGAGTGGGGGTGGCGGATCTGGCGGAGGAGGC
AGCGATATCCAGATGACGCAGTCCCCTAGTTCACTTTCCGCATCCCTGGGGGATC
GGGTTACCATTACATGCCGCGCGTCACAGGGTATCCGGAATGATCTGGGATGGTA
CCAGCAGAAGCCGGGAAAGGCTCCTAAGCGCCTCATCTACGCCAGCTCCACCCT
GCAGAGTGGAGTGCCCTCCCGGTTTTCAGGCAGTGGCTCCGGTACGGAGTTTACT
CTTACAATTAGCAGCCTGCAGCCAGAAGATTTTGCAACTTACTACTGTTTGCAGC
ATAATAATTTCCCCTGGACCTTTGGTCAGGGCACCAAGGTGGAGATCAAAAGAG
CAGCCGCCATCGAAGTAATGTATCCCCCCCCGTACCTTGACAATGAGAAGTCAA
ATGGAACCATTATCCATGTTAAGGGCAAACACCTCTGCCCTTCTCCACTGTTCCCT
GGCCCTAGTAAGCCGTTTTGGGTGCTGGTGGTAGTCGGTGGGGTGCTGGCTTGTT
ACTCTCTTCTCGTGACCGTCGCCTTTATAATCTTTTGGGTCAGATCCAAAAGAAGC
CGCCTGCTCCATAGCGATTACATGAATATGACTCCACGCCGCCCTGGCCCCACAA
GGAAACACTACCAGCCTTACGCACCACCTAGAGATTTCGCTGCCTATCGGAGCCG
AGTGAAATTTTCTAGATCAGCTGATGCTCCCGCCTATCAGCAGGGACAGAATCAA
CTTTACAATGAGCTGAACCTGGGTCGCAGAGAAGAGTACGACGTTTTGGACAAA
CGCCGGGGCCGAGATCCTGAGATGGGGGGGAAGCCGAGAAGGAAGAATCCTCA
AGAAGGCCTGTACAACGAGCTTCAAAAAGACAAAATGGCTGAGGCGTACTCTGA
GATCGGCATGAAGGGCGAGCGGAGACGAGGCAAGGGTCACGATGGCTTGTATCA
GGGCCTGAGTACAGCCACAAAGGACACCTATGACGCCCTCCACATGCAGGCACT
GCCCCCACGCTAG
Construct 10E3 CD28 AA (signal sequence in bold; CDRs underlined)
(SEQ ID NO: 62)
MALPVTALLLPLALLLHAARPQVTLKESGPVLVKPTETLTLTCTVSGFSLINARMG
VSWIRQPPGKALEWLAHIFSNAEKSYRTSLKSRLTISKDTSKSQVVLTMTNMDPVDT
ATYYCARIPGYGGNGDYHYYGMDVWGQGTTVTVSSGGGGSGGGGSGGGGSDIQM
TQSPSSLSASLGDRVTITCRASQGIRNDLGWYQQKPGKAPKRLIYASSTLQSGVPSRF
SGSGSGTEFTLTISSLQPEDFATYYCLQHNNFPWTFGQGTKVEIKRAAAIEVMYPPPY
LDNEKSNGTIIHVKGKHLCPSPLFPGPSKPFWVLVVVGGVLACYSLLVTVAFIIFWVR
SKRSRLLHSDYMNMTPRRPGPTRKHYQPYAPPRDFAAYRSRVKFSRSADAPAYQQG
QNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
Construct 10E3 CD28T DNA (signal sequence in bold)
(SEQ ID NO: 63)
ATGGCACTCCCCGTAACTGCTCTGCTGCTGCCGTTGGCATTGCTCCTGCACG
CCGCACGCCCGCAAGTTACTTTGAAGGAGTCTGGACCTGTACTGGTGAAGCCAA
CCGAGACACTGACACTCACGTGTACAGTGAGTGGTTTTTCCTTGATCAACGCAAG
GATGGGCGTCAGCTGGATCAGGCAACCCCCTGGCAAGGCTCTGGAATGGCTCGC
TCACATATTCAGCAATGCCGAAAAAAGCTACCGGACAAGCCTGAAATCCCGCCT
GACTATTTCCAAGGACACTTCTAAGTCTCAGGTGGTGCTGACCATGACCAACATG
GACCCGGTGGACACCGCCACCTATTACTGCGCAAGAATCCCTGGGTATGGTGGG
AATGGTGACTACCATTATTATGGGATGGATGTGTGGGGGCAAGGCACAACCGTA
ACGGTCTCAAGCGGTGGGGGAGGCTCAGGGGGCGGAGGCTCCGGAGGTGGCGG
CTCCGACATTCAGATGACCCAAAGCCCGTCCAGCCTGTCCGCCAGCCTGGGAGAT
AGAGTGACAATCACGTGTAGAGCTTCCCAAGGGATAAGAAATGATCTCGGGTGG
TATCAGCAGAAGCCCGGCAAAGCCCCCAAAAGGCTTATATATGCTAGTAGTACA
CTGCAGTCTGGAGTTCCTTCCCGATTTTCAGGTAGCGGCTCCGGTACAGAGTTCA
CCCTCACGATAAGCTCACTCCAGCCTGAGGATTTCGCAACGTACTACTGCCTCCA
GCACAACAATTTTCCCTGGACTTTCGGCCAGGGCACCAAGGTGGAGATCAAGAG
GGCCGCTGCCCTTGATAATGAAAAGTCAAACGGAACAATCATTCACGTGAAGGG
CAAGCACCTCTGTCCGTCACCCTTGTTCCCTGGTCCATCCAAGCCATTCTGGGTGT
TGGTCGTAGTGGGTGGAGTCCTCGCTTGTTACTCTCTGCTCGTCACCGTGGCTTTT
ATAATCTTCTGGGTTAGATCCAAAAGAAGCCGCCTGCTCCATAGCGATTACATGA
ATATGACTCCACGCCGCCCTGGCCCCACAAGGAAACACTACCAGCCTTACGCAC
CACCTAGAGATTTCGCTGCCTATCGGAGCCGAGTGAAATTTTCTAGATCAGCTGA
TGCTCCCGCCTATCAGCAGGGACAGAATCAACTTTACAATGAGCTGAACCTGGGT
CGCAGAGAAGAGTACGACGTTTTGGACAAACGCCGGGGCCGAGATCCTGAGATG
GGGGGGAAGCCGAGAAGGAAGAATCCTCAAGAAGGCCTGTACAACGAGCTTCA
AAAAGACAAAATGGCTGAGGCGTACTCTGAGATCGGCATGAAGGGCGAGCGGA
GACGAGGCAAGGGTCACGATGGCTTGTATCAGGGCCTGAGTACAGCCACAAAGG
ACACCTATGACGCCCTCCACATGCAGGCACTGCCCCCACGCTAG
Construct 10E3 CD28T AA (signal sequence in bold; CDRs underlined)
(SEQ ID NO: 64)
MALPVTALLLPLALLLHAARPQVTLKESGPVLVKPTETLTLTCTVSGFSLINARMG
VSWIRQPPGKALEWLAHIFSNAEKSYRTSLKSRLTISKDTSKSQVVLTMTNMDPVDT
ATYYCARIPGYGGNGDYHYYGMDVWGQGTTVTVSSGGGGSGGGGSGGGGSDIQM
TQSPSSLSASLGDRVTITCRASQGIRNDLGWYQQKPGKAPKRLIYASSTLQSGVPSRF
SGSGSGTEFTLTISSLQPEDFATYYCLQHNNFPWTFGQGTKVEIKRAAALDNEKSNGT
IIHVKGKHLCPSPLFPGPSKPFWVLVVVGGVLACYSLLVTVAFIIFWVRSKRSRLLHS
DYMNMTPRRPGPTRKHYQPYAPPRDFAAYRSRVKFSRSADAPAYQQGQNQLYNEL
NLGRREEYDVLDKRRGRDPEMGGKPRRKNPQEGLYNELQKDKMAEAYSEIGMKGE
RRRGKGHDGLYQGLSTATKDTYDALHMQALPPR
Construct 10E3 CD8 DNA (signal sequence in bold)
(SEQ ID NO: 65)
ATGGCACTCCCCGTAACTGCTCTGCTGCTGCCGTTGGCATTGCTCCTGCACG
CCGCACGCCCGCAGGTGACACTCAAGGAATCAGGGCCCGTACTGGTGAAACCT
ACTGAGACCCTGACACTGACTTGCACCGTGTCTGGGTTCTCTCTGATTAACGCTC
GAATGGGTGTGAGTTGGATACGCCAGCCTCCAGGGAAGGCTCTGGAGTGGTTGG
CCCACATTTTCTCCAACGCCGAGAAGAGCTACAGGACTAGTCTGAAGTCCAGACT
TACCATTTCCAAAGACACAAGTAAATCACAGGTGGTGCTGACAATGACAAACAT
GGACCCGGTTGATACTGCTACCTATTATTGTGCCCGCATTCCCGGCTACGGCGGC
AATGGCGACTATCACTATTATGGTATGGATGTCTGGGGGCAGGGGACCACTGTTA
CCGTGTCCAGCGGGGGTGGTGGCAGCGGAGGTGGAGGGAGCGGTGGTGGGGGG
AGTGATATTCAGATGACCCAGAGCCCTAGCTCTCTTTCCGCTTCTCTGGGCGATA
GAGTCACCATCACCTGCCGGGCCTCTCAAGGCATCCGGAACGATCTTGGATGGTA
TCAGCAGAAGCCCGGCAAGGCACCAAAAAGGCTGATCTACGCATCAAGCACCCT
GCAATCTGGGGTGCCGTCCCGGTTTTCTGGTTCTGGTAGTGGGACCGAGTTTACT
CTGACTATTTCTTCCCTGCAGCCTGAGGACTTTGCTACGTACTATTGTCTGCAGCA
TAACAACTTCCCCTGGACGTTCGGGCAGGGTACGAAAGTGGAAATTAAGCGCGC
CGCCGCCCTGTCCAACTCCATTATGTATTTCTCTCATTTTGTCCCAGTGTTCCTGC
CCGCTAAACCCACAACTACTCCGGCGCCCCGACCGCCAACTCCCGCACCTACCAT
CGCAAGCCAGCCATTGAGCCTCCGACCTGAGGCATGTAGACCAGCAGCCGGCGG
TGCCGTGCACACAAGGGGACTGGATTTCGCCTGCGACATATATATTTGGGCCCCT
CTGGCTGGAACCTGTGGGGTTCTGCTGCTCTCTCTCGTTATTACACTGTATTGCAA
TCATCGCAATAGATCCAAAAGAAGCCGCCTGCTCCATAGCGATTACATGAATATG
ACTCCACGCCGCCCTGGCCCCACAAGGAAACACTACCAGCCTTACGCACCACCT
AGAGATTTCGCTGCCTATCGGAGCCGAGTGAAATTTTCTAGATCAGCTGATGCTC
CCGCCTATCAGCAGGGACAGAATCAACTTTACAATGAGCTGAACCTGGGTCGCA
GAGAAGAGTACGACGTTTTGGACAAACGCCGGGGCCGAGATCCTGAGATGGGGG
GGAAGCCGAGAAGGAAGAATCCTCAAGAAGGCCTGTACAACGAGCTTCAAAAA
GACAAAATGGCTGAGGCGTACTCTGAGATCGGCATGAAGGGCGAGCGGAGACG
AGGCAAGGGTCACGATGGCTTGTATCAGGGCCTGAGTACAGCCACAAAGGACAC
CTATGACGCCCTCCACATGCAGGCACTGCCCCCACGCTAG
Construct 10E3 CD8 AA (signal sequence in bold; CDRs underlined)
(SEQ ID NO: 66)
MALPVTALLLPLALLLHAARPQVTLKESGPVLVKPTETLTLTCTVSGFSLINARMG
VSWIRQPPGKALEWLAHIFSNAEKSYRTSLKSRLTISKDTSKSQVVLTMTNMDPVDT
ATYYCARIPGYGGNGDYHYYGMDVWGQGTTVTVSSGGGGSGGGGSGGGGSDIQM
TQSPSSLSASLGDRVTITCRASQGIRNDLGWYQQKPGKAPKRLIYASSTLQSGVPSRF
SGSGSGTEFTLTISSLQPEDFATYYCLQHNNFPWTFGQGTKVEIKRAAALSNSIMYFS
HFVPVFLPAKPTTTPAPRPPTPAPTIASQPLSLRPEACRPAAGGAVHTRGLDFACDIYI
WAPLAGTCGVLLLSLVITLYCNHRNRSKRSRLLHSDYMNMTPRRPGPTRKHYQPYA
PPRDFAAYRSRVKFSRSADAPAYQQGQNQLYNELNLGRREEYDVLDKRRGRDPEM
GGKPRRKNPQEGLYNELQKDKMAEAYSEIGMKGERRRGKGHDGLYQGLSTATKDT
YDALHMQALPPR
Construct 8B5 CD28 DNA (signal sequence in bold)
(SEQ ID NO: 67)
ATGGCACTCCCCGTAACTGCTCTGCTGCTGCCGTTGGCATTGCTCCTGCACG
CCGCACGCCCGCAGATCCAGTTGGTGGAATCAGGGGGCGGTGTGGTGCAGCCG
GGTAGGAGCCTGAGACTGTCATGCGTGGCGTCTGGCTTCACATTCAAGAACTACG
GCATGCACTGGGTGCGACAGGCCCCCGGAAAGGGTTTGGAGTGGGTCGCCGTGA
TCTGGTACGACGGATCTAATGAGTATTACGGAGATCCTGTGAAGGGAAGGTTCA
CCATCTCCCGCGACAATAGCAAAAATATGCTCTACCTGCAAATGAACTCACTCAG
GGCGGATGATACGGCGGTCTACTATTGCGCTCGCTCAGGGATTGCTGTGGCCGGC
GCATTCGATTACTGGGGACAGGGTACCCTGGTGACAGTATCAAGCGGAGGCGGC
GGCTCTGGCGGCGGCGGATCTGGCGGGGGGGGAAGTGAGATTGTGTTGACACAG
TCTCCCGATACCCTGTCACTGTCACCCGGCGAGAAGGCAACGCTGAGTTGCAGA
GCAAGCCAGTCAGTCTCCTCTTCTTTTCTGGCCTGGTATCAGCAAAAACCAGGTC
AGGCACCATCTCTCCTGATTTACGTTGCCAGCAGACGGGCGGCTGGCATTCCCGA
CAGGTTCTCTGGAAGCGGATCTGGGACCGATTTTACCCTGACAATTAGCCGCTTG
GAGCCCGAAGACTTTGGTATGTTTTACTGCCAGCACTACGGAAGGACACCTTTCA
CATTTGGCCCGGGCACGAAAGTCGATATAAAACGCGCAGCCGCCATTGAAGTAA
TGTACCCACCACCTTATTTGGACAATGAAAAGTCCAATGGTACCATTATTCACGT
CAAGGGAAAGCATCTCTGTCCAAGCCCTCTGTTCCCCGGCCCCTCCAAACCATTC
TGGGTGCTGGTGGTCGTCGGCGGAGTTCTGGCCTGCTATTCTCTGCTCGTGACTGT
TGCATTCATCATTTTCTGGGTGAGATCCAAAAGAAGCCGCCTGCTCCATAGCGAT
TACATGAATATGACTCCACGCCGCCCTGGCCCCACAAGGAAACACTACCAGCCTT
ACGCACCACCTAGAGATTTCGCTGCCTATCGGAGCCGAGTGAAATTTTCTAGATC
AGCTGATGCTCCCGCCTATCAGCAGGGACAGAATCAACTTTACAATGAGCTGAA
CCTGGGTCGCAGAGAAGAGTACGACGTTTTGGACAAACGCCGGGGCCGAGATCC
TGAGATGGGGGGGAAGCCGAGAAGGAAGAATCCTCAAGAAGGCCTGTACAACG
AGCTTCAAAAAGACAAAATGGCTGAGGCGTACTCTGAGATCGGCATGAAGGGCG
AGCGGAGACGAGGCAAGGGTCACGATGGCTTGTATCAGGGCCTGAGTACAGCCA
CAAAGGACACCTATGACGCCCTCCACATGCAGGCACTGCCCCCACGCTAG
Construct 8B5 CD28 AA (signal sequence in bold)
(SEQ ID NO: 68)
MALPVTALLLPLALLLHAARPQIQLVESGGGVVQPGRSLRLSCVASGFTFKNYGM
HWVRQAPGKGLEWVAVIWYDGSNEYYGDPVKGRFTISRDNSKNMLYLQMNSLRA
DDTAVYYCARSGIAVAGAFDYWGQGTLVTVSSGGGGSGGGGSGGGGSEIVLTQSPD
TLSLSPGEKATLSCRASQSVSSSFLAWYQQKPGQAPSLLIYVASRRAAGIPDRFSGSG
SGTDFTLTISRLEPEDFGMFYCQHYGRTPFTFGPGTKVDIKRAAAIEVMYPPPYLDNE
KSNGTIIHVKGKHLCPSPLFPGPSKPFWVLVVVGGVLACYSLLVTVAFIIFWVRSKRS
RLLHSDYMNMTPRRPGPTRKHYQPYAPPRDFAAYRSRVKFSRSADAPAYQQGQNQ
LYNELNLGRREEYDVLDKRRGRDPEMGGKPRRKNPQEGLYNELQKDKMAEAYSEI
GMKGERRRGKGHDGLYQGLSTATKDTYDALHMQALPPR
Construct 8B5 CD28T DNA (signal sequence in bold)
(SEQ ID NO: 69)
ATGGCACTCCCCGTAACTGCTCTGCTGCTGCCGTTGGCATTGCTCCTGCACG
CCGCACGCCCGCAGATTCAGCTCGTGGAGTCAGGTGGTGGCGTGGTTCAGCCCG
GACGGTCCCTGCGACTCTCTTGTGTGGCAAGCGGATTTACCTTTAAGAACTATGG
CATGCACTGGGTGAGGCAGGCCCCTGGAAAAGGACTGGAGTGGGTTGCTGTGAT
CTGGTACGACGGGTCCAACGAATATTATGGCGATCCTGTGAAGGGACGGTTTAC
AATCTCACGCGATAACTCAAAGAACATGCTGTACCTGCAAATGAACTCTCTGCGC
GCTGATGACACTGCCGTGTATTATTGCGCTCGGAGTGGTATCGCCGTCGCAGGAG
CATTTGATTATTGGGGGCAAGGGACCCTCGTGACAGTGAGTTCCGGAGGGGGAG
GTTCTGGTGGAGGCGGCTCTGGTGGGGGAGGCAGCGAGATCGTTCTGACCCAGT
CTCCTGACACACTGTCACTGTCCCCTGGTGAAAAGGCCACACTGTCTTGTAGAGC
GTCCCAGAGCGTTTCCAGTTCCTTCCTTGCATGGTATCAACAAAAACCCGGGCAG
GCTCCAAGCTTGCTGATCTACGTGGCCAGCCGCCGGGCCGCAGGCATCCCTGATA
GGTTTAGCGGTTCTGGGAGCGGGACGGACTTCACCTTGACAATATCACGGCTGGA
ACCCGAAGACTTCGGAATGTTTTATTGCCAGCACTACGGAAGAACTCCATTCACC
TTTGGCCCGGGAACGAAGGTAGACATCAAGAGAGCAGCAGCCCTCGACAACGAG
AAATCCAATGGAACCATTATCCATGTGAAGGGGAAACATCTCTGCCCTTCACCAT
TGTTCCCTGGACCCAGCAAGCCTTTTTGGGTTCTGGTCGTGGTGGGGGGCGTCCT
GGCTTGTTACTCCCTCCTCGTTACAGTCGCCTTCATAATCTTTTGGGTTAGATCCA
AAAGAAGCCGCCTGCTCCATAGCGATTACATGAATATGACTCCACGCCGCCCTG
GCCCCACAAGGAAACACTACCAGCCTTACGCACCACCTAGAGATTTCGCTGCCTA
TCGGAGCCGAGTGAAATTTTCTAGATCAGCTGATGCTCCCGCCTATCAGCAGGGA
CAGAATCAACTTTACAATGAGCTGAACCTGGGTCGCAGAGAAGAGTACGACGTT
TTGGACAAACGCCGGGGCCGAGATCCTGAGATGGGGGGGAAGCCGAGAAGGAA
GAATCCTCAAGAAGGCCTGTACAACGAGCTTCAAAAAGACAAAATGGCTGAGGC
GTACTCTGAGATCGGCATGAAGGGCGAGCGGAGACGAGGCAAGGGTCACGATG
GCTTGTATCAGGGCCTGAGTACAGCCACAAAGGACACCTATGACGCCCTCCACA
TGCAGGCACTGCCCCCACGCTAG
Construct 8B5 CD28T AA (signal sequence in bold)
(SEQ ID NO: 70)
MALPVTALLLPLALLLHAARPQIQLVESGGGVVQPGRSLRLSCVASGFTFKNYGM
HWVRQAPGKGLEWVAVIWYDGSNEYYGDPVKGRFTISRDNSKNMLYLQMNSLRA
DDTAVYYCARSGIAVAGAFDYWGQGTLVTVSSGGGGSGGGGSGGGGSEIVLTQSPD
TLSLSPGEKATLSCRASQSVSSSFLAWYQQKPGQAPSLLIYVASRRAAGIPDRFSGSG
SGTDFTLTISRLEPEDFGMFYCQHYGRTPFTFGPGTKVDIKRAAALDNEKSNGTIIHV
KGKHLCPSPLFPGPSKPFWVLVVVGGVLACYSLLVTVAFIIFWVRSKRSRLLHSDYM
NMTPRRPGPTRKHYQPYAPPRDFAAYRSRVKFSRSADAPAYQQGQNQLYNELNLGR
REEYDVLDKRRGRDPEMGGKPRRKNPQEGLYNELQKDKMAEAYSEIGMKGERRRG
KGHDGLYQGLSTATKDTYDALHMQALPPR
Construct 8B5 CD8 DNA (signal sequence in bold)
(SEQ ID NO: 71)
ATGGCACTCCCCGTAACTGCTCTGCTGCTGCCGTTGGCATTGCTCCTGCACG
CCGCACGCCCGCAGATACAGCTTGTCGAATCCGGTGGCGGGGTGGTGCAGCCTG
GACGCAGCCTGCGGCTTTCTTGCGTGGCCAGCGGATTTACCTTCAAGAACTACGG
GATGCATTGGGTCCGCCAGGCACCCGGCAAAGGCCTTGAGTGGGTTGCAGTGAT
CTGGTACGACGGCAGTAACGAGTATTATGGCGACCCCGTGAAGGGAAGGTTTAC
TATTTCAAGAGATAATAGTAAGAACATGTTGTATCTGCAAATGAACAGTCTGAGA
GCGGACGACACTGCCGTGTACTACTGTGCTCGCTCCGGCATCGCTGTGGCAGGGG
CCTTTGACTACTGGGGTCAGGGGACGCTGGTCACGGTTAGTTCCGGGGGCGGTGG
TTCCGGAGGAGGCGGTTCCGGCGGCGGCGGATCAGAAATCGTTCTTACTCAGAG
TCCCGATACGCTGTCCTTGTCTCCGGGAGAAAAAGCCACACTGAGCTGCCGAGCC
TCACAGTCAGTAAGTTCTTCATTCCTCGCCTGGTACCAGCAAAAACCGGGGCAGG
CCCCTTCCCTGCTTATCTACGTGGCCTCTAGGAGAGCCGCCGGTATTCCTGACCG
GTTCAGCGGAAGTGGTTCCGGGACTGATTTTACGCTCACGATCTCCCGATTGGAG
CCCGAGGATTTCGGGATGTTCTACTGTCAGCATTATGGAAGAACGCCCTTTACCT
TCGGTCCGGGAACTAAGGTTGATATTAAGCGGGCTGCTGCCCTTAGCAACTCCAT
CATGTATTTTTCTCACTTCGTGCCAGTATTCCTGCCAGCCAAACCGACCACAACC
CCAGCACCTAGACCTCCTACTCCCGCTCCCACCATAGCTTCACAGCCGCTGAGTT
TGAGGCCAGAGGCCTGTCGGCCTGCTGCAGGCGGAGCAGTTCACACCAGGGGAC
TTGACTTTGCATGTGACATCTATATTTGGGCTCCACTGGCGGGAACCTGCGGGGT
GCTCCTTTTGTCACTCGTTATCACACTGTATTGCAATCATAGGAATAGATCCAAA
AGAAGCCGCCTGCTCCATAGCGATTACATGAATATGACTCCACGCCGCCCTGGCC
CCACAAGGAAACACTACCAGCCTTACGCACCACCTAGAGATTTCGCTGCCTATCG
GAGCCGAGTGAAATTTTCTAGATCAGCTGATGCTCCCGCCTATCAGCAGGGACAG
AATCAACTTTACAATGAGCTGAACCTGGGTCGCAGAGAAGAGTACGACGTTTTG
GACAAACGCCGGGGCCGAGATCCTGAGATGGGGGGGAAGCCGAGAAGGAAGAA
TCCTCAAGAAGGCCTGTACAACGAGCTTCAAAAAGACAAAATGGCTGAGGCGTA
CTCTGAGATCGGCATGAAGGGCGAGCGGAGACGAGGCAAGGGTCACGATGGCTT
GTATCAGGGCCTGAGTACAGCCACAAAGGACACCTATGACGCCCTCCACATGCA
GGCACTGCCCCCACGCTAG
Construct 8B5 CD8 AA (signal sequence in bold)
(SEQ ID NO: 72)
MALPVTALLLPLALLLHAARPQIQLVESGGGVVQPGRSLRLSCVASGFTFKNYGM
HWVRQAPGKGLEWVAVIWYDGSNEYYGDPVKGRFTISRDNSKNMLYLQMNSLRA
DDTAVYYCARSGIAVAGAFDYWGQGTLVTVSSGGGGSGGGGSGGGGSEIVLTQSPD
TLSLSPGEKATLSCRASQSVSSSFLAWYQQKPGQAPSLLIYVASRRAAGIPDRFSGSG
SGTDFTLTISRLEPEDFGMFYCQHYGRTPFTFGPGTKVDIKRAAALSNSIMYFSHFVP
VFLPAKPTTTPAPRPPTPAPTIASQPLSLRPEACRPAAGGAVHTRGLDFACDIYIWAPL
AGTCGVLLLSLVITLYCNHRNRSKRSRLLHSDYMNMTPRRPGPTRKHYQPYAPPRDF
AAYRSRVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDTYDALH
MQALPPR
Construct 4E9 CD28 DNA (signal sequence in bold)
(SEQ ID NO: 73)
ATGGCACTCCCCGTAACTGCTCTGCTGCTGCCGTTGGCATTGCTCCTGCACG
CCGCACGCCCGCAGGTGCAGCTGGTGCAGAGTGGGGCAGAAGTAAAGAAGCCT
GGTGCCTCTGTCAAAGTTAGTTGCAAAGCATCTGGGTATACTTTCACCGGTTACT
ATATCCATTGGGTTCGGCAGGCCCCGGAGCAGGGACTGGAGTGGATGGGCTGGA
TCAACCCAAATTCAGGCGGCACTAACTATGCTCAAAAGTTCCAGGGCAGGGTCA
CAATGGCCCGGGATACTTCAATTAGCACCGTCTATATGGATCTTAGTCGGCTGCG
CAGTGACGATACCGCTGTCTACTATTGCGCAAGGATCAGGGGCGGCAATTCTGTT
TTTGACTATTGGGGCCAGGGAACACTGGTGACCGTCTCCTCTGGTGGAGGCGGTA
GTGGTGGAGGCGGGTCCGGAGGAGGGGGCTCCGATATAGTGATGACTCAAAGTC
CCGATAGCTTGGCAGTATCTCTTGGGGAACGCGCCACTATTAACTGTAAATCCAC
CCAGTCCATTCTCTATACCTCTAACAACAAGAATTTCCTCGCGTGGTATCAGCAA
AAACCCGGGCAGCCACCTAAACTGCTTATATCCTGGGCCAGCATCAGGGAGTCC
GGCGTCCCTGATCGGTTCAGCGGTAGTGGCAGCGGGACAGACTTCGCTCTGACCA
TCAGTAGCCTCCAGGCTGAAGATGTCGCAGTGTATTATTGCCAGCAGTACTTCAG
CACGATGTTTAGCTTCGGGCAGGGAACCAAGCTGGAAATAAAGAGAGCTGCAGC
AATCGAGGTGATGTACCCACCTCCATATCTGGACAATGAAAAGTCCAATGGCACT
ATCATACACGTGAAGGGCAAACACCTGTGTCCATCTCCACTTTTCCCGGGCCCGT
CTAAACCTTTCTGGGTGCTGGTGGTGGTGGGCGGAGTTCTGGCCTGTTATTCACT
GCTGGTCACCGTGGCTTTCATCATTTTTTGGGTAAGATCCAAAAGAAGCCGCCTG
CTCCATAGCGATTACATGAATATGACTCCACGCCGCCCTGGCCCCACAAGGAAA
CACTACCAGCCTTACGCACCACCTAGAGATTTCGCTGCCTATCGGAGCCGAGTGA
AATTTTCTAGATCAGCTGATGCTCCCGCCTATCAGCAGGGACAGAATCAACTTTA
CAATGAGCTGAACCTGGGTCGCAGAGAAGAGTACGACGTTTTGGACAAACGCCG
GGGCCGAGATCCTGAGATGGGGGGGAAGCCGAGAAGGAAGAATCCTCAAGAAG
GCCTGTACAACGAGCTTCAAAAAGACAAAATGGCTGAGGCGTACTCTGAGATCG
GCATGAAGGGCGAGCGGAGACGAGGCAAGGGTCACGATGGCTTGTATCAGGGCC
TGAGTACAGCCACAAAGGACACCTATGACGCCCTCCACATGCAGGCACTGCCCC
CACGCTAG
Construct 4E9 CD28 AA (signal sequence in bold)
(SEQ ID NO: 74)
MALPVTALLLPLALLLHAARPQVQLVQSGAEVKKPGASVKVSCKASGYTFTGYYI
HWVRQAPEQGLEWMGWINPNSGGTNYAQKFQGRVTMARDTSISTVYMDLSRLRSD
DTAVYYCARIRGGNSVFDYWGQGTLVTVSSGGGGSGGGGSGGGGSDIVMTQSPDSL
AVSLGERATINCKSTQSILYTSNNKNFLAWYQQKPGQPPKLLISWASIRESGVPDRFS
GSGSGTDFALTISSLQAEDVAVYYCQQYFSTMFSFGQGTKLEIKRAAAIEVMYPPPYL
DNEKSNGTIIHVKGKHLCPSPLFPGPSKPFWVLVVVGGVLACYSLLVTVAFIIFWVRS
KRSRLLHSDYMNMTPRRPGPTRKHYQPYAPPRDFAAYRSRVKFSRSADAPAYQQGQ
NQLYNELNLGRREEYDVLDKRRGRDPEMGGKPRRKNPQEGLYNELQKDKMAEAYS
EIGMKGERRRGKGHDGLYQGLSTATKDTYDALHMQALPPR
Construct 4E9 CD28T DNA (signal sequence in bold)
(SEQ ID NO: 75)
ATGGCACTCCCCGTAACTGCTCTGCTGCTGCCGTTGGCATTGCTCCTGCACG
CCGCACGCCCGCAGGTACAGCTGGTGCAGAGCGGGGCCGAGGTCAAAAAGCCC
GGGGCTTCAGTTAAGGTTAGCTGCAAGGCTTCCGGCTACACCTTTACCGGTTACT
ATATTCACTGGGTTAGACAGGCACCTGAGCAAGGACTGGAGTGGATGGGGTGGA
TTAACCCCAATAGCGGTGGGACCAACTACGCCCAGAAGTTTCAAGGCCGAGTGA
CAATGGCACGAGACACCTCCATTTCCACTGTGTACATGGACTTGAGCCGCCTCAG
GTCAGACGACACCGCAGTGTACTACTGTGCGCGAATCCGCGGCGGAAACAGCGT
GTTTGACTACTGGGGTCAGGGCACGTTGGTGACCGTGTCTTCCGGAGGGGGGGG
ATCTGGTGGCGGGGGCTCCGGCGGAGGCGGTAGTGATATTGTGATGACTCAGTC
ACCGGACAGTCTTGCTGTTTCACTTGGTGAGAGGGCCACCATAAATTGTAAAAGC
ACCCAGAGCATTCTCTACACATCTAACAACAAAAATTTCCTGGCCTGGTACCAGC
AGAAGCCCGGACAGCCACCCAAATTGCTGATTAGCTGGGCCAGCATTCGAGAAT
CTGGGGTTCCGGACCGCTTTTCCGGGTCTGGCTCTGGGACCGACTTCGCTTTGAC
CATAAGCTCTCTTCAGGCCGAAGACGTCGCAGTATACTATTGTCAACAGTATTTT
TCTACCATGTTTTCCTTCGGCCAGGGAACTAAGTTGGAGATCAAGAGAGCAGCTG
CATTGGATAATGAGAAGTCCAATGGCACTATTATCCACGTGAAAGGTAAACACC
TGTGTCCCTCACCCCTGTTTCCAGGACCTAGTAAACCATTCTGGGTCTTGGTTGTA
GTCGGGGGCGTTTTGGCATGTTATTCCCTTCTTGTGACAGTCGCCTTTATCATTTT
CTGGGTGAGATCCAAAAGAAGCCGCCTGCTCCATAGCGATTACATGAATATGAC
TCCACGCCGCCCTGGCCCCACAAGGAAACACTACCAGCCTTACGCACCACCTAG
AGATTTCGCTGCCTATCGGAGCCGAGTGAAATTTTCTAGATCAGCTGATGCTCCC
GCCTATCAGCAGGGACAGAATCAACTTTACAATGAGCTGAACCTGGGTCGCAGA
GAAGAGTACGACGTTTTGGACAAACGCCGGGGCCGAGATCCTGAGATGGGGGGG
AAGCCGAGAAGGAAGAATCCTCAAGAAGGCCTGTACAACGAGCTTCAAAAAGA
CAAAATGGCTGAGGCGTACTCTGAGATCGGCATGAAGGGCGAGCGGAGACGAG
GCAAGGGTCACGATGGCTTGTATCAGGGCCTGAGTACAGCCACAAAGGACACCT
ATGACGCCCTCCACATGCAGGCACTGCCCCCACGCTAG
Construct 4E9 CD28T AA (signal sequence in bold)
(SEQ ID NO: 76)
MALPVTALLLPLALLLHAARPQVQLVQSGAEVKKPGASVKVSCKASGYTFTGYYI
HWVRQAPEQGLEWMGWINPNSGGTNYAQKFQGRVTMARDTSISTVYMDLSRLRSD
DTAVYYCARIRGGNSVFDYWGQGTLVTVSSGGGGSGGGGSGGGGSDIVMTQSPDSL
AVSLGERATINCKSTQSILYTSNNKNFLAWYQQKPGQPPKLLISWASIRESGVPDRFS
GSGSGTDFALTISSLQAEDVAVYYCQQYFSTMFSFGQGTKLEIKRAAALDNEKSNGT
IIHVKGKHLCPSPLFPGPSKPFWVLVVVGGVLACYSLLVTVAFIIFWVRSKRSRLLHS
DYMNMTPRRPGPTRKHYQPYAPPRDFAAYRSRVKFSRSADAPAYQQGQNQLYNEL
NLGRREEYDVLDKRRGRDPEMGGKPRRKNPQEGLYNELQKDKMAEAYSETGMKGE
RRRGKGHDGLYQGLSTATKDTYDALHMQALPPR
Construct 4E9 CD8 DNA (signal sequence in bold)
(SEQ ID NO: 77)
ATGGCACTCCCCGTAACTGCTCTGCTGCTGCCGTTGGCATTGCTCCTGCACG
CCGCACGCCCGCAAGTTCAGCTTGTGCAGAGCGGAGCTGAGGTGAAAAAACCA
GGCGCCTCCGTTAAGGTGTCTTGCAAAGCCAGCGGATACACATTTACCGGGTACT
ATATTCACTGGGTGAGGCAGGCCCCTGAACAGGGCCTTGAATGGATGGGGTGGA
TCAATCCAAATTCCGGGGGAACCAATTATGCTCAGAAATTTCAGGGCAGAGTGA
CAATGGCCAGGGACACCTCAATCAGCACAGTCTACATGGACCTGAGCCGCCTGA
GGTCTGATGACACAGCCGTCTACTACTGTGCCCGGATCAGAGGGGGAAACAGTG
TCTTCGACTATTGGGGGCAGGGAACCCTGGTGACTGTCTCCTCCGGGGGAGGGG
GTAGCGGGGGAGGCGGCAGCGGCGGGGGTGGTTCTGACATTGTTATGACCCAAT
CCCCAGACTCTCTGGCCGTGAGCCTGGGTGAGAGAGCCACCATCAATTGCAAGT
CCACCCAGAGCATACTCTATACGTCAAACAATAAGAATTTCCTGGCGTGGTATCA
GCAAAAGCCGGGTCAACCACCCAAGTTGTTGATTAGCTGGGCATCAATTCGAGA
ATCTGGCGTCCCTGATAGGTTTAGCGGGAGCGGTAGTGGAACCGACTTTGCGCTG
ACCATTTCATCCCTTCAGGCAGAGGACGTGGCTGTGTATTACTGTCAACAGTACT
TCAGCACGATGTTTTCTTTCGGCCAGGGGACGAAGCTGGAGATAAAGCGGGCCG
CAGCACTCAGCAACAGCATCATGTACTTTTCTCATTTCGTCCCAGTTTTTCTCCCC
GCCAAACCCACCACTACCCCTGCTCCTAGGCCTCCCACTCCCGCACCCACCATTG
CTTCCCAACCTCTGTCATTGAGGCCCGAAGCCTGCAGACCTGCCGCAGGAGGGG
CTGTGCACACCCGCGGTCTGGATTTTGCTTGTGATATCTACATTTGGGCCCCTTTG
GCCGGAACCTGCGGAGTGTTGTTGCTGAGCCTTGTTATCACGTTGTACTGTAATC
ACAGAAACAGATCCAAAAGAAGCCGCCTGCTCCATAGCGATTACATGAATATGA
CTCCACGCCGCCCTGGCCCCACAAGGAAACACTACCAGCCTTACGCACCACCTA
GAGATTTCGCTGCCTATCGGAGCCGAGTGAAATTTTCTAGATCAGCTGATGCTCC
CGCCTATCAGCAGGGACAGAATCAACTTTACAATGAGCTGAACCTGGGTCGCAG
AGAAGAGTACGACGTTTTGGACAAACGCCGGGGCCGAGATCCTGAGATGGGGGG
GAAGCCGAGAAGGAAGAATCCTCAAGAAGGCCTGTACAACGAGCTTCAAAAAG
ACAAAATGGCTGAGGCGTACTCTGAGATCGGCATGAAGGGCGAGCGGAGACGA
GGCAAGGGTCACGATGGCTTGTATCAGGGCCTGAGTACAGCCACAAAGGACACC
TATGACGCCCTCCACATGCAGGCACTGCCCCCACGCTAG
Construct 4E9 CD8 AA (signal sequence in bold)
(SEQ ID NO: 78)
MALPVTALLLPLALLLHAARPQVQLVQSGAEVKKPGASVKVSCKASGYTFTGYYI
HWVRQAPEQGLEWMGWINPNSGGTNYAQKFQGRVTMARDTSISTVYMDLSRLRSD
DTAVYYCARIRGGNSVFDYWGQGTLVTVSSGGGGSGGGGSGGGGSDIVMTQSPDSL
AVSLGERATINCKSTQSILYTSNNKNFLAWYQQKPGQPPKLLISWASIRESGVPDRFS
GSGSGTDFALTISSLQAEDVAVYYCQQYFSTMFSFGQGTKLEIKRAAALSNSIMYFSH
FVPVFLPAKPTTTPAPRPPTPAPTIASQPLSLRPEACRPAAGGAVHTRGLDFACDIYIW
APLAGTCGVLLLSLVITLYCNHRNRSKRSRLLHSDYMNMTPRRPGPTRKHYQPYAPP
RDFAAYRSRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTATKDTYD
ALHMQALPPR
Construct 11F11 CD28 DNA (signal sequence in bold)
(SEQ ID NO: 79)
ATGGCACTCCCCGTAACTGCTCTGCTGCTGCCGTTGGCATTGCTCCTGCACG
CCGCACGCCCGCAGGTGCAGCTCCAAGAGTCAGGACCAGGACTTGTCAAACCA
AGCCAGACCCTCAGCCTTACCTGCACCGTCAGCGGGGGCTCCATCAGCTCTGGGG
CTTACTACTGGACATGGATACGACAGCATCCCGGTAAAGGTCTGGAGTGGATCG
GGTACATACACTATAGTGGTTCCACATATTCTAATCCATCTCTTAAGAGTCGAAT
TACAATTTCACTCGATACTTCAAAGAATCAGTTCAGCTTGAAACTGAACTCCGTG
ACCGCGGCTGACACCGCCGTGTACTACTGTGCACGCCAAGAGGATTATGGCGGA
CTGTTCGATTATTGGGGGCAGGGAACTCTCGTGACAGTGAGCTCCGGCGGGGGC
GGCAGCGGTGGGGGTGGAAGTGGTGGAGGGGGCAGCGAGATCGTGATGACCCA
GAGTCCTGCCACACTGTCAGTGAGTCCTGGGGAGCGAATCACACTTTCCTGTCGA
GCGTCTCAGTCCGTGACCACGGACCTGGCGTGGTACCAGCAGATGCCAGGCCAG
GCGCCAAGACTCCTGATCTACGACGCTTCTACCCGCGCTACTGGTTTCCCCGCCA
GATTCTCCGGAAGCGGGTCCGGGACGGATTTTACACTTACCATCTCTTCATTGCA
GGCTGAGGATTTTGCCGTGTACTACTGTCAGCATTACAAAACCTGGCCCCTCACT
TTCGGGGGCGGAACAAAAGTGGAAATTAAACGGGCAGCAGCTATTGAGGTGATG
TACCCACCCCCCTACCTGGACAACGAGAAATCCAATGGCACCATCATCCACGTTA
AGGGTAAGCACTTGTGTCCCTCACCACTCTTCCCTGGGCCTAGCAAGCCATTCTG
GGTCCTGGTGGTCGTGGGAGGCGTGCTGGCCTGCTATTCCCTCCTGGTTACCGTT
GCCTTTATCATATTTTGGGTCAGATCCAAAAGAAGCCGCCTGCTCCATAGCGATT
ACATGAATATGACTCCACGCCGCCCTGGCCCCACAAGGAAACACTACCAGCCTT
ACGCACCACCTAGAGATTTCGCTGCCTATCGGAGCCGAGTGAAATTTTCTAGATC
AGCTGATGCTCCCGCCTATCAGCAGGGACAGAATCAACTTTACAATGAGCTGAA
CCTGGGTCGCAGAGAAGAGTACGACGTTTTGGACAAACGCCGGGGCCGAGATCC
TGAGATGGGGGGGAAGCCGAGAAGGAAGAATCCTCAAGAAGGCCTGTACAACG
AGCTTCAAAAAGACAAAATGGCTGAGGCGTACTCTGAGATCGGCATGAAGGGCG
AGCGGAGACGAGGCAAGGGTCACGATGGCTTGTATCAGGGCCTGAGTACAGCCA
CAAAGGACACCTATGACGCCCTCCACATGCAGGCACTGCCCCCACGCTAG
Construct 11F11 CD28 AA (signal sequence in bold)
(SEQ ID NO: 80)
MALPVTALLLPLALLLHAARPQVQLQESGPGLVKPSQTLSLTCTVSGGSISSGAYY
WTWIRQHPGKGLEWIGYIHYSGSTYSNPSLKSRITISLDTSKNQFSLKLNSVTAADTA
VYYCARQEDYGGLFDYWGQGTLVTVSSGGGGSGGGGSGGGGSEIVMTQSPATLSV
SPGERITLSCRASQSVTTDLAWYQQMPGQAPRLLIYDASTRATGFPARFSGSGSGTDF
TLTISSLQAEDFAVYYCQHYKTWPLTFGGGTKVEIKRAAAIEVMYPPPYLDNEKSNG
TIIHVKGKHLCPSPLFPGPSKPFWVLVVVGGVLACYSLLVTVAFIIFWVRSKRSRLLH
SDYMNMTPRRPGPTRKHYQPYAPPRDFAAYRSRVKFSRSADAPAYQQGQNQLYNE
LNLGRREEYDVLDKRRGRDPEMGGKPRRKNPQEGLYNELQKDKMAEAYSEIGMKG
ERRRGKGHDGLYQGLSTATKDTYDALHMQALPPR
Construct 11F11 CD28T DNA (signal sequence in bold)
(SEQ ID NO: 81)
ATGGCACTCCCCGTAACTGCTCTGCTGCTGCCGTTGGCATTGCTCCTGCACG
CCGCACGCCCGCAGGTGCAGTTGCAGGAGAGCGGGCCAGGCCTGGTGAAGCCC
AGCCAAACACTGAGCCTCACCTGTACTGTGTCCGGTGGTAGCATTTCCAGCGGGG
CGTATTATTGGACATGGATACGCCAACACCCTGGAAAAGGGTTGGAGTGGATTG
GATACATCCATTATTCTGGGTCCACCTATAGTAACCCTTCTCTCAAGTCTCGCATT
ACTATTAGTTTGGATACCTCTAAGAATCAGTTTAGTCTGAAGCTGAACAGTGTAA
CCGCCGCCGACACCGCGGTCTACTACTGTGCTAGGCAGGAGGATTACGGGGGAC
TGTTCGATTACTGGGGCCAGGGGACATTGGTCACCGTTTCAAGCGGGGGCGGCG
GATCTGGCGGAGGGGGATCTGGAGGCGGAGGCTCTGAGATCGTAATGACTCAGA
GCCCAGCCACCCTGTCCGTCTCTCCCGGCGAACGCATCACTCTGAGCTGTAGGGC
ATCACAGTCTGTTACCACAGATCTGGCTTGGTATCAACAAATGCCTGGGCAGGCC
CCGCGACTGTTGATTTATGACGCCTCTACGCGGGCCACAGGATTTCCTGCCCGGT
TCTCCGGGTCTGGTTCTGGCACCGATTTTACCTTGACAATCAGTAGCTTGCAGGC
AGAAGATTTCGCTGTGTATTACTGCCAACATTATAAGACATGGCCTTTGACATTC
GGCGGGGGAACCAAAGTGGAGATCAAACGCGCCGCAGCCCTGGACAATGAGAA
GTCTAATGGGACCATCATTCACGTCAAAGGGAAACACCTGTGCCCCTCTCCTCTG
TTCCCAGGCCCTTCTAAGCCCTTCTGGGTTCTCGTGGTGGTGGGCGGTGTCCTGGC
CTGCTATTCCCTTCTTGTGACAGTGGCCTTTATCATTTTTTGGGTGAGATCCAAAA
GAAGCCGCCTGCTCCATAGCGATTACATGAATATGACTCCACGCCGCCCTGGCCC
CACAAGGAAACACTACCAGCCTTACGCACCACCTAGAGATTTCGCTGCCTATCGG
AGCCGAGTGAAATTTTCTAGATCAGCTGATGCTCCCGCCTATCAGCAGGGACAGA
ATCAACTTTACAATGAGCTGAACCTGGGTCGCAGAGAAGAGTACGACGTTTTGG
ACAAACGCCGGGGCCGAGATCCTGAGATGGGGGGGAAGCCGAGAAGGAAGAAT
CCTCAAGAAGGCCTGTACAACGAGCTTCAAAAAGACAAAATGGCTGAGGCGTAC
TCTGAGATCGGCATGAAGGGCGAGCGGAGACGAGGCAAGGGTCACGATGGCTTG
TATCAGGGCCTGAGTACAGCCACAAAGGACACCTATGACGCCCTCCACATGCAG
GCACTGCCCCCACGCTAG
Construct 11F11 CD28T AA (signal sequence in bold)
(SEQ ID NO: 82)
MALPVTALLLPLALLLHAARPQVQLQESGPGLVKPSQTLSLTCTVSGGSISSGAYY
WTWIRQHPGKGLEWIGYIHYSGSTYSNPSLKSRITISLDTSKNQFSLKLNSVTAADTA
VYYCARQEDYGGLFDYWGQGTLVTVSSGGGGSGGGGSGGGGSEIVMTQSPATLSV
SPGER1TLSCRASQSVTTDLAWYQQMPGQAPRLL1YDASTRATGFPARFSGSGSGTDF
TLTISSLQAEDFAVYYCQHYKTWPLTFGGGTKVEIKRAAALDNEKSNGTIIHVKGKH
LCPSPLFPGPSKPFWVLVVVGGVLACYSLLVTVAFIIFWVRSKRSRLLHSDYMNMTP
RRPGPTRKHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERRRGKGH
DGLYQGLSTATKDTYDALHMQALPPR
Construct 11F11 CD8 DNA (signal sequence in bold)
(SEQ ID NO: 83)
ATGGCACTCCCCGTAACTGCTCTGCTGCTGCCGTTGGCATTGCTCCTGCACG
CCGCACGCCCGCAGGTACAGTTGCAGGAAAGCGGCCCCGGCCTTGTAAAACCA
AGCCAGACTCTCAGTTTGACTTGCACCGTCTCAGGAGGAAGCATTTCCAGTGGGG
CTTATTATTGGACTTGGATTCGGCAGCATCCTGGGAAAGGGTTGGAATGGATCGG
TTATATTCATTATAGCGGTAGCACCTATTCCAATCCGTCTTTGAAAAGCAGAATC
ACTATTTCACTCGACACCTCTAAGAACCAGTTCAGTCTCAAACTGAACTCCGTGA
CAGCGGCCGACACAGCTGTGTACTACTGTGCACGGCAAGAAGATTATGGGGGGC
TGTTCGATTATTGGGGCCAAGGCACACTGGTGACAGTATCAAGCGGTGGAGGAG
GCTCCGGGGGCGGAGGAAGTGGAGGCGGGGGGAGCGAAATTGTGATGACCCAG
TCTCCAGCCACGCTGTCAGTGTCTCCGGGAGAACGCATAACCCTCTCCTGCCGGG
CCAGTCAGTCCGTCACGACCGATTTGGCTTGGTATCAACAGATGCCTGGGCAGGC
CCCCCGCTTGCTGATCTATGACGCCTCCACCAGAGCAACTGGTTTCCCCGCCCGG
TTCAGCGGATCTGGAAGCGGTACAGATTTTACACTTACCATCTCATCATTGCAAG
CTGAGGATTTTGCCGTGTACTACTGCCAGCACTACAAGACCTGGCCTTTGACGTT
CGGCGGCGGAACAAAAGTGGAGATTAAAAGAGCCGCTGCCCTCAGTAACTCAAT
CATGTACTTTAGTCACTTTGTGCCTGTGTTTCTGCCAGCAAAGCCAACAACCACA
CCAGCACCCCGCCCTCCAACGCCTGCCCCAACCATCGCCTCCCAGCCTCTGAGCT
TGAGGCCTGAGGCTTGTCGCCCAGCTGCTGGAGGTGCTGTGCATACACGAGGACT
GGATTTCGCCTGCGATATCTATATCTGGGCACCACTTGCCGGTACTTGTGGTGTGT
TGCTGCTCTCACTGGTCATCACGCTGTACTGTAACCATAGGAATAGATCCAAAAG
AAGCCGCCTGCTCCATAGCGATTACATGAATATGACTCCACGCCGCCCTGGCCCC
ACAAGGAAACACTACCAGCCTTACGCACCACCTAGAGATTTCGCTGCCTATCGG
AGCCGAGTGAAATTTTCTAGATCAGCTGATGCTCCCGCCTATCAGCAGGGACAGA
ATCAACTTTACAATGAGCTGAACCTGGGTCGCAGAGAAGAGTACGACGTTTTGG
ACAAACGCCGGGGCCGAGATCCTGAGATGGGGGGGAAGCCGAGAAGGAAGAAT
CCTCAAGAAGGCCTGTACAACGAGCTTCAAAAAGACAAAATGGCTGAGGCGTAC
TCTGAGATCGGCATGAAGGGCGAGCGGAGACGAGGCAAGGGTCACGATGGCTTG
TATCAGGGCCTGAGTACAGCCACAAAGGACACCTATGACGCCCTCCACATGCAG
GCACTGCCCCCACGCTAG
Construct 11F11 CD8 AA (signal sequence in bold)
(SEQ ID NO: 84)
MALPVTALLLPLALLLHAARPQVQLQESGPGLVKPSQTLSLTCTVSGGSISSGAYY
WTWIRQHPGKGLEWIGYIHYSGSTYSNPSLKSRITISLDTSKNQFSLKLNSVTAADTA
VYYCARQEDYGGLFDYWGQGTLVTVSSGGGGSGGGGSGGGGSEIVMTQSPATLSV
SPGERITLSCRASQSVTTDLAWYQQMPGQAPRLLIYDASTRATGFPARFSGSGSGTDF
TLTISSLQAEDFAVYYCQHYKTWPLTFGGGTKVEIKRAAALSNSIMYFSHFVPVFLPA
KPTTTPAPRPPTPAPTIASQPLSLRPEACRPAAGGAVHTRGLDFACDIYIWAPLAGTC
GVLLLSLVITLYCNHRNRSKRSRLLHSDYMNMTPRRPGPTRKHYQPYAPPRDFAAY
RSRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQ
ALPPR
Human FLT3 NM_004119 AA
(SEQ ID NO: 85)
MPALARDGGQLPLLVVFSAMIFGTITNQDLPVIKCVLINHKNNDSSVGKSS
SYPMVSESPEDLGCALRPQSSGTVYEAAAVEVDVSASITLQVLVDAPGNISCLWVFK
HSSLNCQPHFDLQNRGVVSMVILKMTETQAGEYLLFIQSEATNYTILFTVSIRNTLLY
TLRRPYFRKMENQDALVCISESVPEPIVEWVLCDSQGESCKEESPAVVKKEEKVLHE
LFGTDIRCCARNELGRECTRLFTIDLNQTPQTTLPQLFLKVGEPLWIRCKAVHVNHGF
GLTWELENKALEEGNYFEMSTYSTNRTMIRILFAFVSSVARNDTGYYTCSSSKHPSQ
SALVTIVEKGFINATNSSEDYEIDQYEEFCFSVRFKAYPQIRCTWTFSRKSFPCEQKGL
DNGYSISKFCNHKHQPGEYIFHAENDDAQFTKMFTLNIRRKPQVLAEASASQASCFS
DGYPLPSWTWKKCSDKSPNCTEEITEGVWNRKANRKVFGQWVSSSTLNMSEAIKGF
LVKCCAYNSLGTSCETILLNSPGPFPFIQDNISFYATIGVCLLFIVVLTLLICHKYKKQF
RYESQLQMVQVTGSSDNEYFYVDFREYEYDLKWEFPRENLEFGKVLGSGAFGKVM
NATAYGISKTGVSIQVAVKMLKEKADSSEREALMSELKMMTQLGSHENIVNLLGAC
TLSGPIYLIFEYCCYGDLLNYLRSKREKFHRTWTEIFKEHNFSFYPTFQSHPNSSMPGS
REVQIHPDSDQISGLHGNSFHSEDEIEYENQKRLEEEEDLNVLTFEDLLCFAYQVAKG
MEFLEFKSCVHRDLAARNVLVTHGKVVKICDFGLARDIMSDSNYVVRGNARLPVK
WMAPESLFEGIYTIKSDVWSYGILLWEIFSLGVNPYPGIPVDANFYKLIQNGFKMDQP
FYATEEIYIIMQSCWAFDSRKRPSFPNLTSFLGCQLADAEEAMYQNVDGRVSECPHT
YQNRRPFSREMDLGLLSPQAQVEDS
CAR Signal Peptide DNA
(SEQ ID NO: 86)
ATGGCACTCCCCGTAACTGCTCTGCTGCTGCCGTTGGCATTGCTCCTGCACGCCG
CACGCCCG
CAR Signal Peptide:
(SEQ ID NO: 87)
MALPVTALLLPLALLLHAARP
scFv G4S linker DNA
(SEQ ID NO: 88)
GGCGGTGGAGGCTCCGGAGGGGGGGGCTCTGGCGGAGGGGGCTCC
scFv G4s linker:
(SEQ ID NO: 89)
GGGGSGGGGSGGGGS
scFv Whitlow linker DNA
(SEQ ID NO: 90)
GGGTCTACATCCGGCTCCGGGAAGCCCGGAAGTGGCGAAGGTAGTACAAAGGGG
scFv Whitlow linker:
(SEQ ID NO: 91)
GSTSGSGKPGSGEGSTKG
4-1BB Nucleic Acid Sequence (intracellular domain)
(SEQ ID NO: 92)
AAGCGCGGCAGGAAGAAGCTCCTCTACATTTTTAAGCAGCCTTTTATGAGGCCCG
TACAGACAACACAGGAGGAAGATGGCTGTAGCTGCAGATTTCCCGAGGAGGAGG
AAGGTGGGTGCGAGCTG
4-1BB AA (intracellular domain)
(SEQ ID NO: 93)
KRGRKKLLYIFKQPFMRPVQTTQEEDGCSCRFPEEEEGGCEL
OX40 AA
(SEQ ID NO: 94)
RRDQRLPPDAHKPPGGGSFRTPIQEEQADAHSTLAKI

### INCORPORATION BY REFERENCE

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

### EQUIVALENTS

The foregoing written specification is considered to be sufficient to enable one skilled in the art to practice the invention. The foregoing description and examples detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof

The following examples, including the experiments conducted and results achieved, are provided for illustrative purposes only and are not to be construed as limiting the present invention.

### EXAMPLE 1

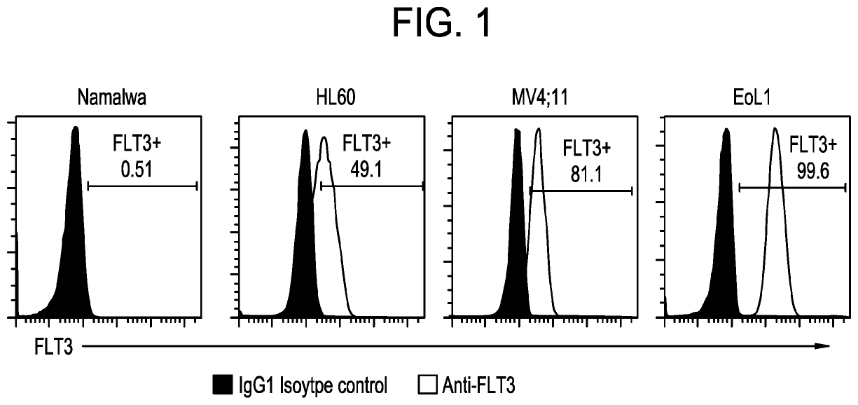

Namalwa, MV4;11, and HL60 cells (ATCC) and EoL1 cells (Sigma-Aldrich) were cultured in RPMI1640 (Lonza)+10% FBS (Coming)+1× Penicillin Streptomycin L-Glutamine (Corning) (R10) medium and maintained at a cell density between 0.5-2.0×106 cells/ml. To examine cell surface FLT3 expression, cells were incubated with an anti-FLT3 antibody (BD Pharmingen) or an IgG1 isotype control antibody (BD Pharmingen) in stain buffer (BD Pharmingen) for 30 minutes at 4° C. Cells were then washed and resuspended in stain buffer with propidium iodide (BD Pharmingen) prior to data acquisition. FLT3 expression on target cells is shown in FIG. 1.

### EXAMPLE 2

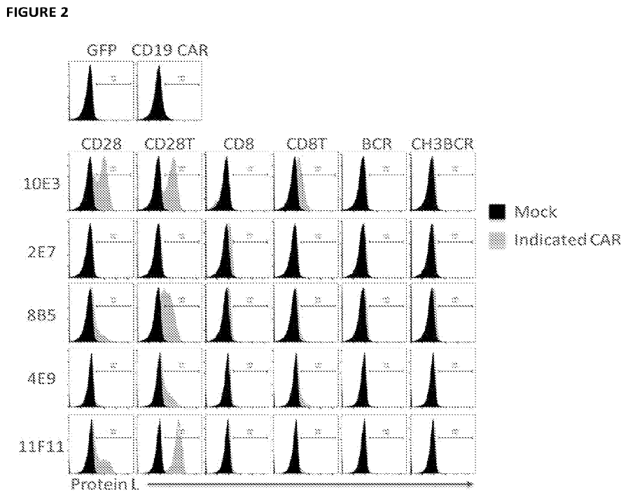

Plasmids encoding a T7 promoter, CAR construct and a beta globin stabilizing sequence were linearized by overnight digestion of 10 μg DNA with EcoRI and BamHI (NEB). DNA was then digested for 2 hours at 50° C. with proteinase K (Thermo Fisher, 600 U/ml) purified with phenol/chloroform and precipitated by adding sodium acetate and two volumes of ethanol. Pellets were then dried, resuspended in RNAse/DNAse-free water and quantified using NanoDrop. One μg of the linear DNA was then used for in vitro transcription using the mMESSAGE mMACHINE T7 Ultra (Thermo Fisher) following the manufacturer's instructions. RNA was further purified using the MEGAClear Kit (Thermo Fisher) following the manufacturer's instructions and quantified using NanoDrop. mRNA integrity was assesed using mobility on an agarose gel. PBMCs were isolated from healthy donor leukopaks (Hemacare) using ficoll-paque density centrifugation per manufacturer's instructions. PBMCs were stimulated using OKT3 (50 ng/ml, Miltenyi Biotec) in R10 medium+TL-2 (300 IU/ml, Proleukin®, Prometheus® Therapeutics and Diagnostics). Seven days post-stimulation, T cells were washed twice in Opti-MEM medium (Thermo Fisher Scientific) and resuspended at a final concentration of 2.5×107 cells/ml in Opti-MEM medium. Ten μg of mRNA was used per electroporation. Electroporation of cells was performed using a Gemini X2 system (Harvard Apparatus BTX) to deliver a single 400 V pulse for 0.5 ms in 2 mm cuvettes (Harvard Apparatus BTX). Cells were immediately transferred to R10+IL-2 medium and allowed to recover for 6 hours. To examine CAR expression, T cells were stained with FLT-=3-HIS (Sino Biological Inc.) or biotinylated Protein L (Thermo Scientific) in stain buffer (BD Pharmingen) for 30 minutes at 4° C. Cells were then washed and stained with anti-HIS-PE (Miltenyi Biotec) or PE Streptavidin (BD Pharmingen) in stain buffer for 30 minutes at 4° C. Cells were then washed and resuspended in stain buffer with propidium iodide (BD Pharmingen) prior to data acquisition. Expression of FLT3 CARs in electroporated T cells is shown in FIG. 2.

### EXAMPLE 3

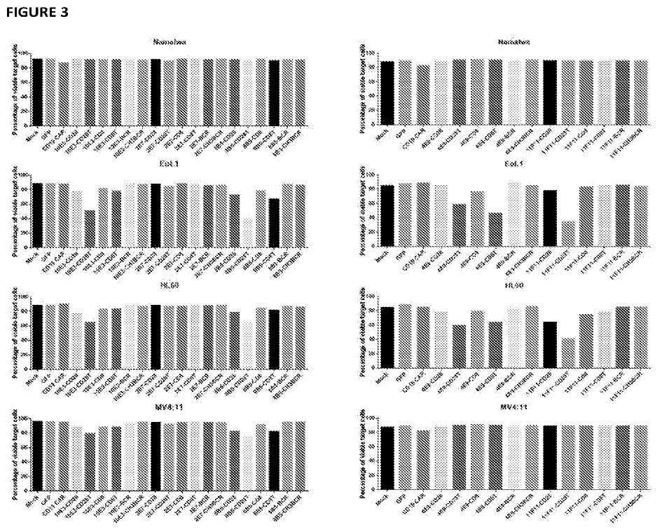

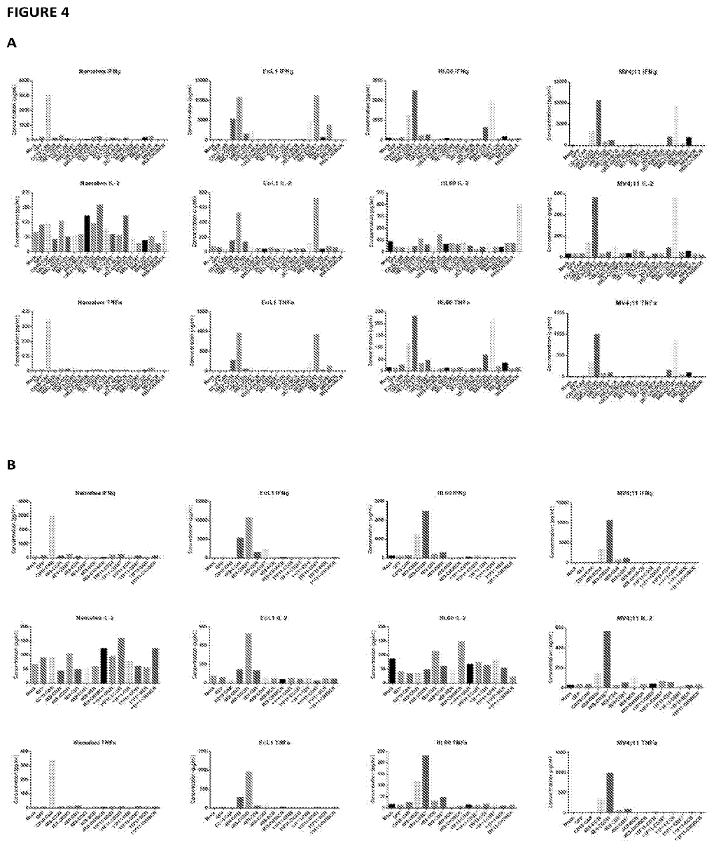

To examine cytolytic activity in electroporated FLT3 CAR T cells, effector cells were cultured with target cells at a 1:1 E:T ratio in R10 medium. Sixteen hours post-coculture, supernatants were analyzed by Luminex (EMD Millipore) and target cell viability was assessed by flow cytometric analysis of propidium iodide (PI) uptake by CD3-negative cells. Cytolytic activity of electroporated CART cells is shown in FIG. 3 and cytokine production is shown in FIG. 4.

### EXAMPLE 4

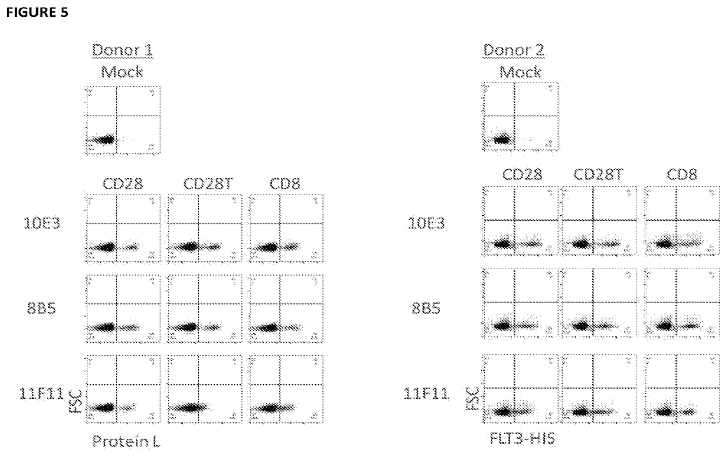

A third generation lentiviral transfer vector containing the different CAR constructs was used along with the ViraPower Lentiviral Packaging Mix (Life Technologies) to generate the lentiviral supernatants. Briefly, a transfection mix was generated by mixing 15 μg of DNA and 22.5 μl of polyethileneimine (Polysciences, 1 mg/ml) in 600 μl of OptiMEM medium. The mix was incubated for 5 minutes at room temperature. Simultaneously, 293T cells (ATCC) were trypsinized, counted and a total of 10×106 total cells were plated in a T75 flask along the transfection mix. Three days after the transfection, supernatants were collected and filtered through a 0.45 μm filter and stored at −80° C. until used. PBMCs were isolated from healthy donor leukopaks (Hemacare) using ficoll-paque density centrifugation per manufacturer's instructions. PBMCs were stimulated using OKT3 (50 ng/ml, Miltenyi Biotec) in R10 medium+IL-2 (300 IU/ml, Proleukin®, Prometheus® Therapeutics and Diagnostics). Forty eight hours post-stimulation, cells were transduced using lentivirus at an MOI=10. Cells were maintained at 0.5-2.0×106 cells/ml prior to use in activity assays. To examine CAR expression, T cells were stained with FLT-3-HIS (Sino Biological Inc.) or biotinylated Protein L (Thermo Scientific) in stain buffer (BD Pharmingen) for 30 minutes at 4° C. Cells were then washed and stained with anti-HIS-PE (Miltenyi Biotec) or PE Streptavidin (BD Pharmingen) in stain buffer for 30 minutes at 4° C. Cells were then washed and resuspended in stain buffer with propidium iodide (BD Pharmingen) prior to data acquisition. Expression of FLT3 CARs in T cells from two healthy donors is shown in FIG. 5.

### EXAMPLE 5

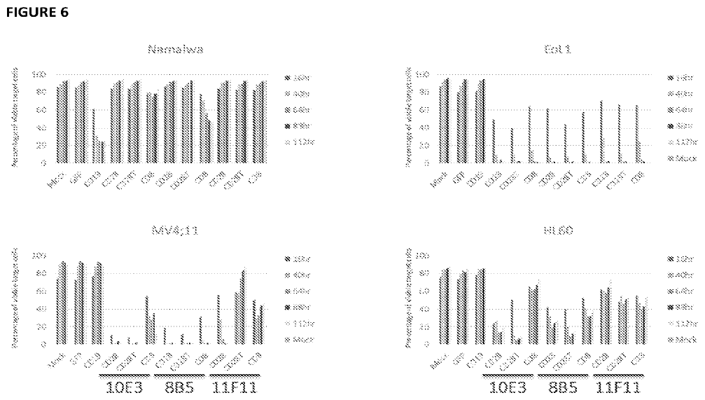

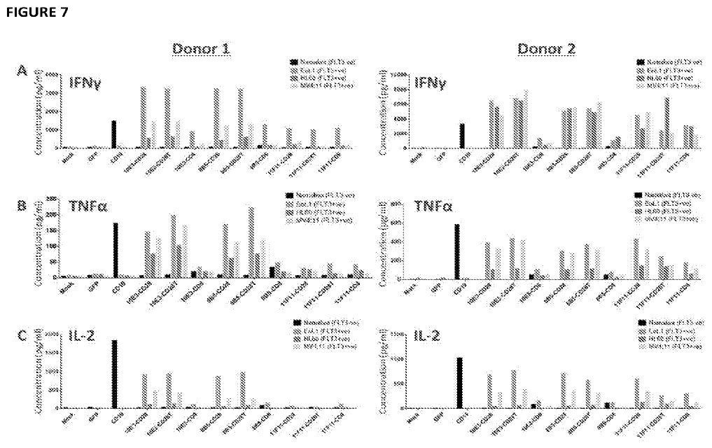

To examine cytolytic activity in lentivirus-transduced FLT3 CAR T cells, effector cells were cultured with target cells at a 1:1 E:T ratio in R10 medium. Sixteen hours post-coculture, supernatants were analyzed by Luminex (EMD Millipore) and target cell viability was assessed by flow cytometric analysis of propidium iodide (PI) uptake by CD3-negative cells. Average cytolytic activity of lentivirus-transduced CAR T cells from two healthy donors is shown in FIG. 6 and cytokine production by CAR T cells from each healthy donor is shown in FIG. 7.

### EXAMPLE 6

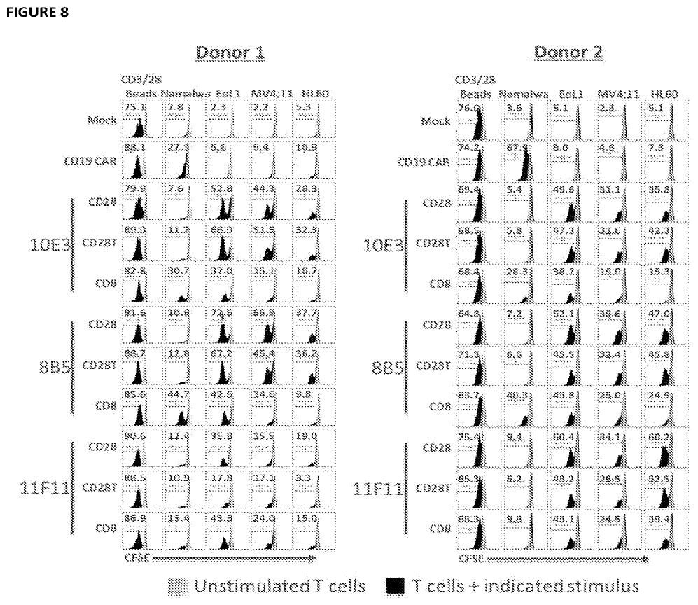

To assess CAR T cell proliferation in response to FLT3 expressing target cells, T cells were labeled with CFSE prior to co-culture with target cells at a 1:1 E:T ratio in R10 medium.Five days later, T cell proliferation was assessed by flow cytometric analysis of CFSE dilution. Proliferation of FLT3 CART cells is shown in FIG. 8.

### EXAMPLE 7

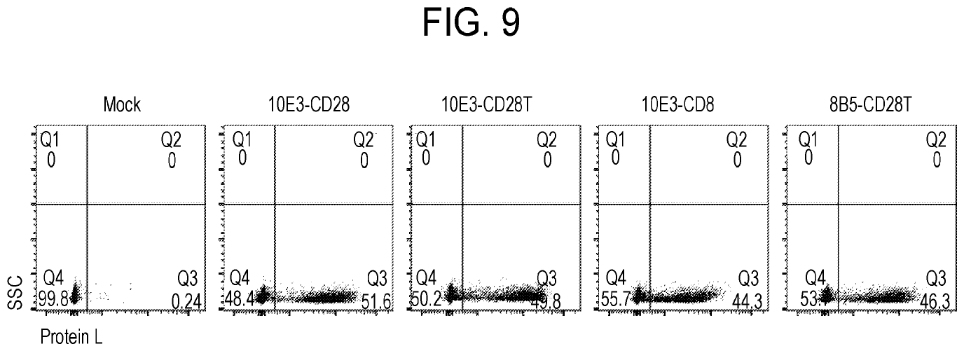

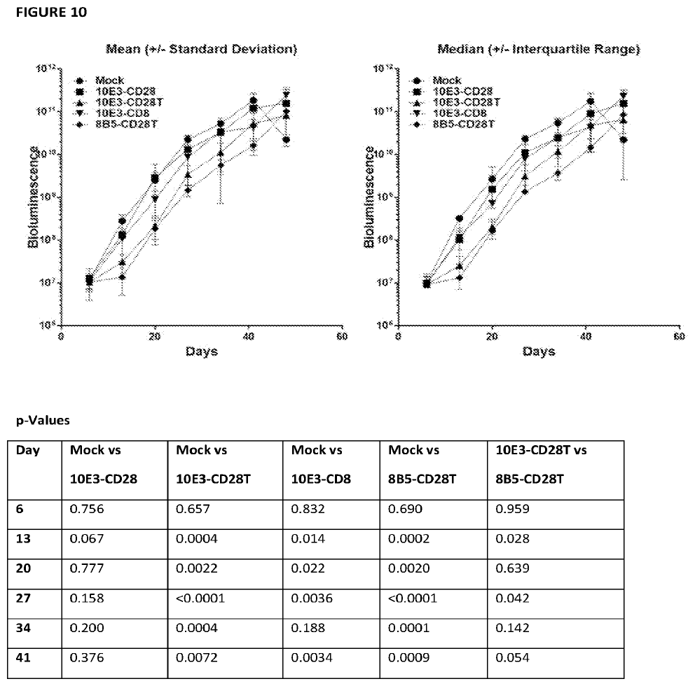

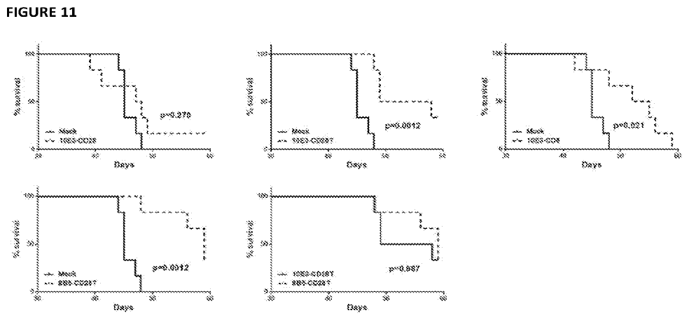

To examine in vivo anti-leukemic activity, FLT3 CAR T cells were generated for use in a xenogeneic model of human AML. CAR expression of the various effector lines used in the xenogeneic model of human AML are shown in FIG. 9. Luciferase-labeled MV4; 11 cells (2×106/animal) were injected intravenously into 5 to 6 week-old female NSG mice. After 6 days, 6×106 T cells (˜50% CAR+) in 200 μl PBS were injected intravenously and the tumor burden of the animals was measured weekly using bioluminescence imaging. As shown in FIG. 10, injection of 10E3-CD28T and 8B5-CD28T expressing CAR T cells significantly reduced the tumor burden at all time points examined. As shown in FIG. 11, this was further confirmed with survival analysis where injection of the 10E3-CD28T or 8B5-CD28T expressing CAR T cells conferred a significant survival advantage over animals that received mock transduced cells or CART cells expressing the 10E3-CD28 or 10E3-CD8 constructs. No significant differences were observed between the 10E3-CD28T and 8B5-CD28T constructs in terms of efficacy.

1. A chimeric antigen receptor comprising an antigen binding molecule that specifically binds to FLT3, wherein the antigen binding molecule comprises:
a) a variable heavy chain CDR1 comprising an amino acid sequence differing by not more than 3, 2, 1, or 0 amino acid residues from that of SEQ ID NO: 17;
b) a variable heavy chain CDR2 comprising an amino acid sequence differing by not more than 3, 2, 1, or 0 amino acid residues from that of SEQ ID NO:18 or;
c) a variable heavy chain CDR3 comprising an amino acid sequence differing by not more than 3, 2, 1, or 0 amino acid residues from that of SEQ ID NOs SEQ ID NO: 19;
d) a variable light chain CDR1 comprising an amino acid sequence differing by not more than 3, 2, 1, or 0 amino acid residues from that of SEQ ID NO:22;
e) a variable light chain CDR2 comprising an amino acid sequence differing by not more than 3, 2, 1, or 0 amino acid residues from that of SEQ ID NO:23;
f) a variable light chain CDR3 comprising an amino acid sequence differing by not more than 3, 2, 1, or 0 amino acid residues from that of SEQ ID:24; or
a variable heavy chain sequence differing by not more than 10, 9, 8, 7, 6, 5, 4, 3, 2, 1, or 0 residues from the variable heavy chain sequence of SEQ ID NO:16; and
a variable light chain sequence differing by not more than 10, 9, 8, 7, 6, 5, 4, 3, 2, 1, or 0 residues from the variable light chain sequence of SEQ ID NO:21.

2. The chimeric antigen receptor according to claim 1 further comprising at least one costimulatory domain.

3. The chimeric antigen receptor according to claim 1 further comprising at least one activating domain.

4. The chimeric antigen receptor according to claim 2 wherein the costimulatory domain is a signaling region of CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1 (CDlla/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CDlld, ITGAE, CD103, ITGAL, CDlla, LFA-1, ITGAM, CDllb, ITGAX, CDllc, ITGBl, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

5. The chimeric antigen receptor according to claim 4 wherein the costimulatory domain comprises CD28.

6. The chimeric antigen receptor according to claim 5 wherein the CD28 costimulatory domain comprises a sequence that differs at no more than 10, 9, 8, 7, 6, 5, 4, 3, 2, 1, or 0 amino acid residues from the sequence of SEQ ID NO: 2, SEQ ID NO: 4, SEQ ID NO: 6, or SEQ ID NO: 8.

7. The chimeric antigen receptor according to claim 3 wherein the CD8 costimulatory domain comprises a sequence that differs at no more than 10, 9, 8, 7, 6, 5, 4, 3, 2, 1, or 0 amino acid residues from the sequence of SEQ ID NO: 14.

8. The chimeric antigen receptor according to claim 3 wherein the activating domain comprises CD3.

9. The chimeric antigen receptor according to claim 7 wherein the CD3 comprises CD3 zeta.

10. The chimeric antigen receptor according to claim 8 wherein the CD3 zeta comprises a sequence that differs at no more than 10, 9, 8, 7, 6, 5, 4, 3, 2, 1, or 0 amino acid residues from the sequence of SEQ ID NO: 10.

11. The chimeric antigen receptor according to claim 1 wherein the costimulatory domain comprises a sequence that differs at no more than 10, 9, 8, 7, 6, 5, 4, 3, 2, 1, or 0 amino acid residues from the sequence of SEQ ID NO: 2 and the activating domain comprises a sequence that differs at no more than 10, 9, 8, 7, 6, 5, 4, 3, 2, 1, or 0 amino acid residues from the sequence of SEQ ID NO: 10.

12-21. (canceled)

22. A chimeric antigen receptor comprising:
(a) a VH region of SEQ ID NO:16 and a VL region of SEQ ID NO:21;
wherein the VH and VL region is linked by at least one linker.

23. The chimeric antigen receptor according to claim 22, wherein the linker comprises the scFv G45 linker or the scFv Whitlow linker.

24-78. (canceled)

79. A chimeric antigen receptor comprising SEQ ID NO:62 or 64.

80. The chimeric antigen receptor of claim 79, wherein the chimeric antigen receptor comprises SEQ ID NO:64.


SEQUENCE LISTING (98 sequences)

SEQ ID NO: 1 - DNA, 294 bp, Homo sapiens
cttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagc

SEQ ID NO: 2 - protein, 98 aa, Homo sapiens
LDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - DNA, 90 bp, Homo sapiens
cttgataatgaaaagtcaaacggaacaatcattcacgtgaagggcaagcacctctgtccgtcacccttgttccctggtccatccaagcca

SEQ ID NO: 4 - protein, 30 aa, Homo sapiens
LDNEKSNGTIIHVKGKHLCPSPLFPGPSKP

SEQ ID NO: 5 - DNA, 81 bp, Homo sapiens
ttctgggtgttggtcgtagtgggtggagtcctcgcttgttactctctgctcgtcaccgtggcttttataatcttctgggtt

SEQ ID NO: 6 - protein, 27 aa, Homo sapiens
FWVLVVVGGVLACYSLLVTVAFIIFWV

SEQ ID NO: 7 - DNA, 123 bp, Homo sapiens
agatccaaaagaagccgcctgctccatagcgattacatgaatatgactccacgccgccctggccccacaaggaaacactaccagccttacgcaccacctagagatttcgctgcctatcggagc

SEQ ID NO: 8 - protein, 41 aa, Homo sapiens
RSKRSRLLHSDYMNMTPRRPGPTRKHYQPYAPPRDFAAYRS

SEQ ID NO: 9 - DNA, 336 bp, Homo sapiens
agggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 10 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 117 bp, Homo sapiens
attgaggtgatgtatccaccgccttacctggataacgaaaagagtaacggtaccatcattcacgtgaaaggtaaacacctgtgtccttctcccctcttccccgggccatcaaagccc

SEQ ID NO: 12 - protein, 39 aa, Homo sapiens
IEVMYPPPYLDNEKSNGTIIHVKGKHLCPSPLFPGPSKP

SEQ ID NO: 13 - DNA, 288 bp, Homo sapiens
gctgcagcattgagcaactcaataatgtattttagtcactttgtaccagtgttcttgccggctaagcctactaccacacccgctccacggccacctaccccagctcctaccatcgcttcacagcctctgtccctgcgcccagaggcttgccgaccggccgcagggggcgctgttcataccagaggactggatttcgcctgcgatatctatatctgggcacccctggccggaacctgcggcgtactcctgctgtccctggtcatcacgctctattgtaatcacaggaac

SEQ ID NO: 14 - protein, 96 aa, Homo sapiens
AAALSNSIMYFSHFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 15 - DNA, 381 bp, Homo sapiens
caggtcaccttgaaggagtctggtcctgtgctggtgaaacccacagagaccctcacgctgacctgcaccgtctctgggttctcactcatcaatgctagaatgggtgtgagctggatccgtcagcccccagggaaggccctggagtggcttgcacacattttttcgaatgccgaaaaatcgtacaggacatctctgaagagcaggctcaccatctccaaggacacctccaaaagccaggtggtccttaccatgaccaacatggaccctgtggacacagccacatattactgtgcacggataccaggctacggtggtaacggggactaccactactacggtatggacgtctggggccaagggaccacggtcaccgtctcctca

SEQ ID NO: 16 - protein, 127 aa, Homo sapiens
QVTLKESGPVLVKPTETLTLTCTVSGFSLINARMGVSWIRQPPGKALEWLAHIFSNAEKSYRTSLKSRLTISKDTSKSQVVLTMTNMDPVDTATYYCARIPGYGGNGDYHYYGMDVWGQGTTVTVSS

SEQ ID NO: 17 - protein, 7 aa, Homo sapiens
NARMGVS

SEQ ID NO: 18 - protein, 16 aa, Homo sapiens
HIFSNAEKSYRTSLKS

SEQ ID NO: 19 - protein, 17 aa, Homo sapiens
IPGYGGNGDYHYYGMDV

SEQ ID NO: 20 - DNA, 324 bp, Homo sapiens
gacatccagatgacccagtctccatcctccctgtctgcatctctaggagacagagtcaccatcacttgccgggcaagtcagggcattagaaatgatttaggctggtatcagcagaaaccagggaaagcccctaagcgcctgatctatgcttcatccactttgcaaagtggggtcccatcaaggttcagcggcagtggatctgggacagagttcactctcacaatcagcagcctgcagcctgaagattttgcaacttattactgtctacagcataataatttcccgtggacgttcggtcagggaacgaaggtggaaatcaaacga

SEQ ID NO: 21 - protein, 108 aa, Homo sapiens
DIQMTQSPSSLSASLGDRVTITCRASQGIRNDLGWYQQKPGKAPKRLIYASSTLQSGVPSRFSGSGSGTEFTLTISSLQPEDFATYYCLQHNNFPWTFGQGTKVEIKR

SEQ ID NO: 22 - protein, 11 aa, Homo sapiens
RASQGIRNDLG

SEQ ID NO: 23 - protein, 7 aa, Homo sapiens
ASSTLQS

SEQ ID NO: 24 - protein, 9 aa, Homo sapiens
LQHNNFPWT

SEQ ID NO: 25 - DNA, 375 bp, Homo sapiens
caggtcaccttgaaggagtctggtcctgtgctggtgaaacccacagagaccctcacgctgacctgcaccgtctctgggttctcactcaggaatgctagaatgggtgtaagctggatccgtcagcctcccgggaaggccctggagtggcttgcacacattttttcgaatgacgaaaaaacctacagcacatctctgaagagcaggctcaccatctccagggacacctccaaaggccaggtggtccttaccatgaccaagatggaccctgtggacacagccacatattactgtgcacggataccctactatggttcggggagtcataactacggtatggacgtctggggccaagggaccacggtcaccgtctcctca

SEQ ID NO: 26 - protein, 125 aa, Homo sapiens
QVTLKESGPVLVKPTETLTLTCTVSGFSLRNARMGVSWIRQPPGKALEWLAHIFSNDEKTYSTSLKSRLTISRDTSKGQVVLTMTKMDPVDTATYYCARIPYYGSGSHNYGMDVWGQGTTVTVSS

SEQ ID NO: 27 - protein, 15 aa, Homo sapiens
IPYYGSGSHNYGMDV

SEQ ID NO: 28 - DNA, 324 bp, Homo sapiens
gacatccagatgacccagtctccatcctccctgtctgcatctgtaggagacagagtcaccatcacttgccgggcaagtcaggacattagaaatgatttcggctggtatcaacagaaaccagggaaagcccctcagcgcctgctctatgctgcatccactttgcaaagtggggtcccatcaaggttcagcggcagtggatctgggacagaattcactctcacaatcagcagcctgcagcctgaagattttgcaacttattactgtctacagtataatacttacccgtggacgttcggtcagggaacgaaggtggaaatcaaacga

SEQ ID NO: 29 - protein, 108 aa, Homo sapiens
DIQMTQSPSSLSASVGDRVTITCRASQDIRNDFGWYQQKPGKAPQRLLYAASTLQSGVPSRFSGSGSGTEFTLTISSLQPEDFATYYCLQYNTYPWTFGQGTKVEIKR

SEQ ID NO: 30 - protein, 11 aa, Homo sapiens
RASQDIRNDFG

SEQ ID NO: 31 - protein, 7 aa, Homo sapiens
AASTLQS

SEQ ID NO: 32 - protein, 9 aa, Homo sapiens
LQYNTYPWT

SEQ ID NO: 33 - DNA, 360 bp, Homo sapiens
cagatacaactggtggagtctgggggaggcgtggtccagcctgggaggtccctgagactctcctgtgtagcgtctggattcaccttcaagaactatggcatgcactgggtccgccaggctccaggcaaggggctggagtgggtggcagttatttggtatgatggaagtaatgaatactatggagaccccgtgaagggccgattcaccatctccagagacaactccaagaacatgttgtatctgcaaatgaacagcctgagagccgatgacacggctgtgtattactgtgcgaggtcgggaatagcagtggctggggcctttgactactggggccagggaaccctggtcaccgtctcctca

SEQ ID NO: 34 - protein, 120 aa, Homo sapiens
QIQLVESGGGVVQPGRSLRLSCVASGFTFKNYGMHWVRQAPGKGLEWVAVIWYDGSNEYYGDPVKGRFTISRDNSKNMLYLQMNSLRADDTAVYYCARSGIAVAGAFDYWGQGTLVTVSS

SEQ ID NO: 35 - protein, 17 aa, Homo sapiens
VIWYDGSNEYYGDPVKG

SEQ ID NO: 36 - protein, 11 aa, Homo sapiens
SGIAVAGAFDY

SEQ ID NO: 37 - DNA, 327 bp, Homo sapiens
gaaattgtgttgacgcagtctccagacaccctgtctttgtctccaggggaaaaagccaccctctcctgcagggccagtcagagtgttagcagcagcttcttggcctggtaccagcagaaacctggacaggctcccagtctcctcatctatgttgcatccagaagggccgctggcatccctgacaggttcagtggcagtgggtctgggacagacttcactctcaccatcagcagactggagcctgaagattttggaatgttttactgtcaacactatggtaggacaccattcactttcggccctgggaccaaagtggatatcaaacga

SEQ ID NO: 38 - protein, 12 aa, Homo sapiens
RASQSVSSSFLA

SEQ ID NO: 39 - protein, 7 aa, Homo sapiens
VASRRAA

SEQ ID NO: 40 - protein, 9 aa, Homo sapiens
QHYGRTPFT

SEQ ID NO: 41 - protein, 109 aa, Homo sapiens
EIVLTQSPDTLSLSPGEKATLSCRASQSVSSSFLAWYQQKPGQAPSLLIYVASRRAAGIPDRFSGSGSGTDFTLTISRLEPEDFGMFYCQHYGRTPFTFGPGTKVDIKR

SEQ ID NO: 42 - protein, 119 aa, Homo sapiens
QVQLVQSGAEVKKPGASVKVSCKASGYTFTGYYIHWVRQAPEQGLEWMGWINPNSGGTNYAQKFQGRVTMARDTSISTVYMDLSRLRSDDTAVYYCARIRGGNSVFDYWGQGTLVTVSS

SEQ ID NO: 43 - protein, 5 aa, Homo sapiens
GYYIH

SEQ ID NO: 44 - protein, 17 aa, Homo sapiens
WINPNSGGTNYAQKFQG

SEQ ID NO: 45 - protein, 10 aa, Homo sapiens
IRGGNSVFDY

SEQ ID NO: 46 - DNA, 342 bp, Homo sapiens
gacatcgtgatgacccagtctccagactccctggctgtgtctctgggcgagagggccaccatcaactgcaagtccacccagagtattttatacacctccaacaataagaacttcttagcttggtaccagcagaaaccagggcagcctcctaaactgctcatttcctgggcatctatccgggaatccggggtccctgaccgattcagtggcagcgggtctgggacagatttcgctctcaccatcagcagcctgcaggctgaagatgtggcagtttattactgtcaacaatattttagtactatgttcagttttggccaggggaccaagctggagatcaaacga

SEQ ID NO: 47 - protein, 114 aa, Homo sapiens
DIVMTQSPDSLAVSLGERATINCKSTQSILYTSNNKNFLAWYQQKPGQPPKLLISWASIRESGVPDRFSGSGSGTDFALTISSLQAEDVAVYYCQQYFSTMFSFGQGTKLEIKR

SEQ ID NO: 48 - protein, 17 aa, Homo sapiens
KSTQSILYTSNNKNFLA

SEQ ID NO: 49 - protein, 7 aa, Homo sapiens
WASIRES

SEQ ID NO: 50 - protein, 9 aa, Homo sapiens
QQYFSTMFS

SEQ ID NO: 51 - DNA, 360 bp, Homo sapiens
caggtgcagctgcaggagtcgggcccaggactggtgaagccttcacagaccctgtccctcacctgcactgtctctggtggctccatcagtagtggtgcatactactggacttggatccgccagcacccagggaagggcctggagtggattgggtacatccattacagtgggagcacctactccaacccgtccctcaagagtcgaattaccatatcgttagacacgtctaagaaccagttctccctgaagctgaactctgtgactgccgcggacacggccgtgtattactgtgcgagacaagaggactacggtggtttgtttgactactggggccagggaaccctggtcaccgtttcctca

SEQ ID NO: 52 - protein, 120 aa, Homo sapiens
QVQLQESGPGLVKPSQTLSLTCTVSGGSISSGAYYWTWIRQHPGKGLEWIGYIHYSGSTYSNPSLKSRITISLDTSKNQFSLKLNSVTAADTAVYYCARQEDYGGLFDYWGQGTLVTVSS

SEQ ID NO: 53 - protein, 7 aa, Homo sapiens
SGAYYWT

SEQ ID NO: 54 - protein, 16 aa, Homo sapiens
YIHYSGSTYSNPSLKS

SEQ ID NO: 55 - protein, 10 aa, Homo sapiens
QEDYGGLFDY

SEQ ID NO: 56 - DNA, 324 bp, Homo sapiens
gaaatagtgatgacgcagtctccagccaccctgtctgtgtctccaggggaaagaatcaccctctcctgcagggccagtcagagtgttaccaccgacttagcctggtaccagcagatgcctggacaggctccccggctcctcatctatgatgcttccaccagggccactggtttcccagccagattcagtggcagtgggtctgggacagacttcacgctcaccatcagcagcctgcaggctgaagattttgcagtttattactgtcaacattataaaacctggcctctcactttcggcggagggactaaggtggagatcaaacga

SEQ ID NO: 57 - protein, 108 aa, Homo sapiens
EIVMTQSPATLSVSPGERITLSCRASQSVTTDLAWYQQMPGQAPRLLIYDASTRATGFPARFSGSGSGTDFTLTISSLQAEDFAVYYCQHYKTWPLTFGGGTKVEIKR

SEQ ID NO: 58 - protein, 11 aa, Homo sapiens
RASQSVTTDLA

SEQ ID NO: 59 - protein, 7 aa, Homo sapiens
DASTRAT

SEQ ID NO: 60 - protein, 9 aa, Homo sapiens
QHYKTWPLT

SEQ ID NO: 61 - DNA, 1482 bp, Homo sapiens
atggcactccccgtaactgctctgctgctgccgttggcattgctcctgcacgccgcacgcccgcaggtgaccctcaaagagtctggacccgtgctcgtaaaacctacggagaccctgacactcacctgcacagtctccggcttcagcctcatcaatgccaggatgggagtttcctggatcaggcaaccgcccggaaaggccctggaatggctcgcacatattttcagtaacgctgaaaaaagctatcggacttctctgaaaagtcggctcacgattagtaaggacacatccaagagccaagtggtgcttacgatgactaacatggaccctgtggatactgcaacctattactgtgctcgaatccctggttatggcggaaatggggactaccactactacggtatggatgtctggggccaagggaccacggttactgtttcaagcggagggggagggagtgggggtggcggatctggcggaggaggcagcgatatccagatgacgcagtcccctagttcactttccgcatccctgggggatcgggttaccattacatgccgcgcgtcacagggtatccggaatgatctgggatggtaccagcagaagccgggaaaggctcctaagcgcctcatctacgccagctccaccctgcagagtggagtgccctcccggttttcaggcagtggctccggtacggagtttactcttacaattagcagcctgcagccagaagattttgcaacttactactgtttgcagcataataatttcccctggacctttggtcagggcaccaaggtggagatcaaaagagcagccgccatcgaagtaatgtatccccccccgtaccttgacaatgagaagtcaaatggaaccattatccatgttaagggcaaacacctctgcccttctccactgttccctggccctagtaagccgttttgggtgctggtggtagtcggtggggtgctggcttgttactctcttctcgtgaccgtcgcctttataatcttttgggtcagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 62 - protein, 493 aa, Homo sapiens
MALPVTALLLPLALLLHAARPQVTLKESGPVLVKPTETLTLTCTVSGFSLINARMGVSWIRQPPGKALEWLAHIFSNAEKSYRTSLKSRLTISKDTSKSQVVLTMTNMDPVDTATYYCARIPGYGGNGDYHYYGMDVWGQGTTVTVSSGGGGSGGGGSGGGGSDIQMTQSPSSLSASLGDRVTITCRASQGIRNDLGWYQQKPGKAPKRLIYASSTLQSGVPSRFSGSGSGTEFTLTISSLQPEDFATYYCLQHNNFPWTFGQGTKVEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - DNA, 1455 bp, Homo sapiens
atggcactccccgtaactgctctgctgctgccgttggcattgctcctgcacgccgcacgcccgcaagttactttgaaggagtctggacctgtactggtgaagccaaccgagacactgacactcacgtgtacagtgagtggtttttccttgatcaacgcaaggatgggcgtcagctggatcaggcaaccccctggcaaggctctggaatggctcgctcacatattcagcaatgccgaaaaaagctaccggacaagcctgaaatcccgcctgactatttccaaggacacttctaagtctcaggtggtgctgaccatgaccaacatggacccggtggacaccgccacctattactgcgcaagaatccctgggtatggtgggaatggtgactaccattattatgggatggatgtgtgggggcaaggcacaaccgtaacggtctcaagcggtgggggaggctcagggggcggaggctccggaggtggcggctccgacattcagatgacccaaagcccgtccagcctgtccgccagcctgggagatagagtgacaatcacgtgtagagcttcccaagggataagaaatgatctcgggtggtatcagcagaagcccggcaaagcccccaaaaggcttatatatgctagtagtacactgcagtctggagttccttcccgattttcaggtagcggctccggtacagagttcaccctcacgataagctcactccagcctgaggatttcgcaacgtactactgcctccagcacaacaattttccctggactttcggccagggcaccaaggtggagatcaagagggccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 64 - protein, 484 aa, Homo sapiens
MALPVTALLLPLALLLHAARPQVTLKESGPVLVKPTETLTLTCTVSGFSLINARMGVSWIRQPPGKALEWLAHIFSNAEKSYRTSLKSRLTISKDTSKSQVVLTMTNMDPVDTATYYCARIPGYGGNGDYHYYGMDVWGQGTTVTVSSGGGGSGGGGSGGGGSDIQMTQSPSSLSASLGDRVTITCRASQGIRNDLGWYQQKPGKAPKRLIYASSTLQSGVPSRFSGSGSGTEFTLTISSLQPEDFATYYCLQHNNFPWTFGQGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - DNA, 1563 bp, Homo sapiens
atggcactccccgtaactgctctgctgctgccgttggcattgctcctgcacgccgcacgcccgcaggtgacactcaaggaatcagggcccgtactggtgaaacctactgagaccctgacactgacttgcaccgtgtctgggttctctctgattaacgctcgaatgggtgtgagttggatacgccagcctccagggaaggctctggagtggttggcccacattttctccaacgccgagaagagctacaggactagtctgaagtccagacttaccatttccaaagacacaagtaaatcacaggtggtgctgacaatgacaaacatggacccggttgatactgctacctattattgtgcccgcattcccggctacggcggcaatggcgactatcactattatggtatggatgtctgggggcaggggaccactgttaccgtgtccagcgggggtggtggcagcggaggtggagggagcggtggtggggggagtgatattcagatgacccagagccctagctctctttccgcttctctgggcgatagagtcaccatcacctgccgggcctctcaaggcatccggaacgatcttggatggtatcagcagaagcccggcaaggcaccaaaaaggctgatctacgcatcaagcaccctgcaatctggggtgccgtcccggttttctggttctggtagtgggaccgagtttactctgactatttcttccctgcagcctgaggactttgctacgtactattgtctgcagcataacaacttcccctggacgttcgggcagggtacgaaagtggaaattaagcgcgccgccgccctgtccaactccattatgtatttctctcattttgtcccagtgttcctgcccgctaaacccacaactactccggcgccccgaccgccaactcccgcacctaccatcgcaagccagccattgagcctccgacctgaggcatgtagaccagcagccggcggtgccgtgcacacaaggggactggatttcgcctgcgacatatatatttgggcccctctggctggaacctgtggggttctgctgctctctctcgttattacactgtattgcaatcatcgcaatagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 66 - protein, 520 aa, Homo sapiens
MALPVTALLLPLALLLHAARPQVTLKESGPVLVKPTETLTLTCTVSGFSLINARMGVSWIRQPPGKALEWLAHIFSNAEKSYRTSLKSRLTISKDTSKSQVVLTMTNMDPVDTATYYCARIPGYGGNGDYHYYGMDVWGQGTTVTVSSGGGGSGGGGSGGGGSDIQMTQSPSSLSASLGDRVTITCRASQGIRNDLGWYQQKPGKAPKRLIYASSTLQSGVPSRFSGSGSGTEFTLTISSLQPEDFATYYCLQHNNFPWTFGQGTKVEIKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - DNA, 1464 bp, Homo sapiens
atggcactccccgtaactgctctgctgctgccgttggcattgctcctgcacgccgcacgcccgcagatccagttggtggaatcagggggcggtgtggtgcagccgggtaggagcctgagactgtcatgcgtggcgtctggcttcacattcaagaactacggcatgcactgggtgcgacaggcccccggaaagggtttggagtgggtcgccgtgatctggtacgacggatctaatgagtattacggagatcctgtgaagggaaggttcaccatctcccgcgacaatagcaaaaatatgctctacctgcaaatgaactcactcagggcggatgatacggcggtctactattgcgctcgctcagggattgctgtggccggcgcattcgattactggggacagggtaccctggtgacagtatcaagcggaggcggcggctctggcggcggcggatctggcggggggggaagtgagattgtgttgacacagtctcccgataccctgtcactgtcacccggcgagaaggcaacgctgagttgcagagcaagccagtcagtctcctcttcttttctggcctggtatcagcaaaaaccaggtcaggcaccatctctcctgatttacgttgccagcagacgggcggctggcattcccgacaggttctctggaagcggatctgggaccgattttaccctgacaattagccgcttggagcccgaagactttggtatgttttactgccagcactacggaaggacacctttcacatttggcccgggcacgaaagtcgatataaaacgcgcagccgccattgaagtaatgtacccaccaccttatttggacaatgaaaagtccaatggtaccattattcacgtcaagggaaagcatctctgtccaagccctctgttccccggcccctccaaaccattctgggtgctggtggtcgtcggcggagttctggcctgctattctctgctcgtgactgttgcattcatcattttctgggtgagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 68 - protein, 487 aa, Homo sapiens
MALPVTALLLPLALLLHAARPQIQLVESGGGVVQPGRSLRLSCVASGFTFKNYGMHWVRQAPGKGLEWVAVIWYDGSNEYYGDPVKGRFTISRDNSKNMLYLQMNSLRADDTAVYYCARSGIAVAGAFDYWGQGTLVTVSSGGGGSGGGGSGGGGSEIVLTQSPDTLSLSPGEKATLSCRASQSVSSSFLAWYQQKPGQAPSLLIYVASRRAAGIPDRFSGSGSGTDFTLTISRLEPEDFGMFYCQHYGRTPFTFGPGTKVDIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - DNA, 1437 bp, Homo sapiens
atggcactccccgtaactgctctgctgctgccgttggcattgctcctgcacgccgcacgcccgcagattcagctcgtggagtcaggtggtggcgtggttcagcccggacggtccctgcgactctcttgtgtggcaagcggatttacctttaagaactatggcatgcactgggtgaggcaggcccctggaaaaggactggagtgggttgctgtgatctggtacgacgggtccaacgaatattatggcgatcctgtgaagggacggtttacaatctcacgcgataactcaaagaacatgctgtacctgcaaatgaactctctgcgcgctgatgacactgccgtgtattattgcgctcggagtggtatcgccgtcgcaggagcatttgattattgggggcaagggaccctcgtgacagtgagttccggagggggaggttctggtggaggcggctctggtgggggaggcagcgagatcgttctgacccagtctcctgacacactgtcactgtcccctggtgaaaaggccacactgtcttgtagagcgtcccagagcgtttccagttccttccttgcatggtatcaacaaaaacccgggcaggctccaagcttgctgatctacgtggccagccgccgggccgcaggcatccctgataggtttagcggttctgggagcgggacggacttcaccttgacaatatcacggctggaacccgaagacttcggaatgttttattgccagcactacggaagaactccattcacctttggcccgggaacgaaggtagacatcaagagagcagcagccctcgacaacgagaaatccaatggaaccattatccatgtgaaggggaaacatctctgcccttcaccattgttccctggacccagcaagcctttttgggttctggtcgtggtggggggcgtcctggcttgttactccctcctcgttacagtcgccttcataatcttttgggttagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 70 - protein, 478 aa, Homo sapiens
MALPVTALLLPLALLLHAARPQIQLVESGGGVVQPGRSLRLSCVASGFTFKNYGMHWVRQAPGKGLEWVAVIWYDGSNEYYGDPVKGRFTISRDNSKNMLYLQMNSLRADDTAVYYCARSGIAVAGAFDYWGQGTLVTVSSGGGGSGGGGSGGGGSEIVLTQSPDTLSLSPGEKATLSCRASQSVSSSFLAWYQQKPGQAPSLLIYVASRRAAGIPDRFSGSGSGTDFTLTISRLEPEDFGMFYCQHYGRTPFTFGPGTKVDIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - DNA, 1545 bp, Homo sapiens
atggcactccccgtaactgctctgctgctgccgttggcattgctcctgcacgccgcacgcccgcagatacagcttgtcgaatccggtggcggggtggtgcagcctggacgcagcctgcggctttcttgcgtggccagcggatttaccttcaagaactacgggatgcattgggtccgccaggcacccggcaaaggccttgagtgggttgcagtgatctggtacgacggcagtaacgagtattatggcgaccccgtgaagggaaggtttactatttcaagagataatagtaagaacatgttgtatctgcaaatgaacagtctgagagcggacgacactgccgtgtactactgtgctcgctccggcatcgctgtggcaggggcctttgactactggggtcaggggacgctggtcacggttagttccgggggcggtggttccggaggaggcggttccggcggcggcggatcagaaatcgttcttactcagagtcccgatacgctgtccttgtctccgggagaaaaagccacactgagctgccgagcctcacagtcagtaagttcttcattcctcgcctggtaccagcaaaaaccggggcaggccccttccctgcttatctacgtggcctctaggagagccgccggtattcctgaccggttcagcggaagtggttccgggactgattttacgctcacgatctcccgattggagcccgaggatttcgggatgttctactgtcagcattatggaagaacgccctttaccttcggtccgggaactaaggttgatattaagcgggctgctgcccttagcaactccatcatgtatttttctcacttcgtgccagtattcctgccagccaaaccgaccacaaccccagcacctagacctcctactcccgctcccaccatagcttcacagccgctgagtttgaggccagaggcctgtcggcctgctgcaggcggagcagttcacaccaggggacttgactttgcatgtgacatctatatttgggctccactggcgggaacctgcggggtgctccttttgtcactcgttatcacactgtattgcaatcataggaatagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 72 - protein, 514 aa, Homo sapiens
MALPVTALLLPLALLLHAARPQIQLVESGGGVVQPGRSLRLSCVASGFTFKNYGMHWVRQAPGKGLEWVAVIWYDGSNEYYGDPVKGRFTISRDNSKNMLYLQMNSLRADDTAVYYCARSGIAVAGAFDYWGQGTLVTVSSGGGGSGGGGSGGGGSEIVLTQSPDTLSLSPGEKATLSCRASQSVSSSFLAWYQQKPGQAPSLLIYVASRRAAGIPDRFSGSGSGTDFTLTISRLEPEDFGMFYCQHYGRTPFTFGPGTKVDIKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - DNA, 1476 bp, Homo sapiens
atggcactccccgtaactgctctgctgctgccgttggcattgctcctgcacgccgcacgcccgcaggtgcagctggtgcagagtggggcagaagtaaagaagcctggtgcctctgtcaaagttagttgcaaagcatctgggtatactttcaccggttactatatccattgggttcggcaggccccggagcagggactggagtggatgggctggatcaacccaaattcaggcggcactaactatgctcaaaagttccagggcagggtcacaatggcccgggatacttcaattagcaccgtctatatggatcttagtcggctgcgcagtgacgataccgctgtctactattgcgcaaggatcaggggcggcaattctgtttttgactattggggccagggaacactggtgaccgtctcctctggtggaggcggtagtggtggaggcgggtccggaggagggggctccgatatagtgatgactcaaagtcccgatagcttggcagtatctcttggggaacgcgccactattaactgtaaatccacccagtccattctctatacctctaacaacaagaatttcctcgcgtggtatcagcaaaaacccgggcagccacctaaactgcttatatcctgggccagcatcagggagtccggcgtccctgatcggttcagcggtagtggcagcgggacagacttcgctctgaccatcagtagcctccaggctgaagatgtcgcagtgtattattgccagcagtacttcagcacgatgtttagcttcgggcagggaaccaagctggaaataaagagagctgcagcaatcgaggtgatgtacccacctccatatctggacaatgaaaagtccaatggcactatcatacacgtgaagggcaaacacctgtgtccatctccacttttcccgggcccgtctaaacctttctgggtgctggtggtggtgggcggagttctggcctgttattcactgctggtcaccgtggctttcatcattttttgggtaagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 74 - protein, 491 aa, Homo sapiens
MALPVTALLLPLALLLHAARPQVQLVQSGAEVKKPGASVKVSCKASGYTFTGYYIHWVRQAPEQGLEWMGWINPNSGGTNYAQKFQGRVTMARDTSISTVYMDLSRLRSDDTAVYYCARIRGGNSVFDYWGQGTLVTVSSGGGGSGGGGSGGGGSDIVMTQSPDSLAVSLGERATINCKSTQSILYTSNNKNFLAWYQQKPGQPPKLLISWASIRESGVPDRFSGSGSGTDFALTISSLQAEDVAVYYCQQYFSTMFSFGQGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - DNA, 1449 bp, Homo sapiens
atggcactccccgtaactgctctgctgctgccgttggcattgctcctgcacgccgcacgcccgcaggtacagctggtgcagagcggggccgaggtcaaaaagcccggggcttcagttaaggttagctgcaaggcttccggctacacctttaccggttactatattcactgggttagacaggcacctgagcaaggactggagtggatggggtggattaaccccaatagcggtgggaccaactacgcccagaagtttcaaggccgagtgacaatggcacgagacacctccatttccactgtgtacatggacttgagccgcctcaggtcagacgacaccgcagtgtactactgtgcgcgaatccgcggcggaaacagcgtgtttgactactggggtcagggcacgttggtgaccgtgtcttccggaggggggggatctggtggcgggggctccggcggaggcggtagtgatattgtgatgactcagtcaccggacagtcttgctgtttcacttggtgagagggccaccataaattgtaaaagcacccagagcattctctacacatctaacaacaaaaatttcctggcctggtaccagcagaagcccggacagccacccaaattgctgattagctgggccagcattcgagaatctggggttccggaccgcttttccgggtctggctctgggaccgacttcgctttgaccataagctctcttcaggccgaagacgtcgcagtatactattgtcaacagtatttttctaccatgttttccttcggccagggaactaagttggagatcaagagagcagctgcattggataatgagaagtccaatggcactattatccacgtgaaaggtaaacacctgtgtccctcacccctgtttccaggacctagtaaaccattctgggtcttggttgtagtcgggggcgttttggcatgttattcccttcttgtgacagtcgcctttatcattttctgggtgagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 76 - protein, 482 aa, Homo sapiens
MALPVTALLLPLALLLHAARPQVQLVQSGAEVKKPGASVKVSCKASGYTFTGYYIHWVRQAPEQGLEWMGWINPNSGGTNYAQKFQGRVTMARDTSISTVYMDLSRLRSDDTAVYYCARIRGGNSVFDYWGQGTLVTVSSGGGGSGGGGSGGGGSDIVMTQSPDSLAVSLGERATINCKSTQSILYTSNNKNFLAWYQQKPGQPPKLLISWASIRESGVPDRFSGSGSGTDFALTISSLQAEDVAVYYCQQYFSTMFSFGQGTKLEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - DNA, 1557 bp, Homo sapiens
atggcactccccgtaactgctctgctgctgccgttggcattgctcctgcacgccgcacgcccgcaagttcagcttgtgcagagcggagctgaggtgaaaaaaccaggcgcctccgttaaggtgtcttgcaaagccagcggatacacatttaccgggtactatattcactgggtgaggcaggcccctgaacagggccttgaatggatggggtggatcaatccaaattccgggggaaccaattatgctcagaaatttcagggcagagtgacaatggccagggacacctcaatcagcacagtctacatggacctgagccgcctgaggtctgatgacacagccgtctactactgtgcccggatcagagggggaaacagtgtcttcgactattgggggcagggaaccctggtgactgtctcctccgggggagggggtagcgggggaggcggcagcggcgggggtggttctgacattgttatgacccaatccccagactctctggccgtgagcctgggtgagagagccaccatcaattgcaagtccacccagagcatactctatacgtcaaacaataagaatttcctggcgtggtatcagcaaaagccgggtcaaccacccaagttgttgattagctgggcatcaattcgagaatctggcgtccctgataggtttagcgggagcggtagtggaaccgactttgcgctgaccatttcatcccttcaggcagaggacgtggctgtgtattactgtcaacagtacttcagcacgatgttttctttcggccaggggacgaagctggagataaagcgggccgcagcactcagcaacagcatcatgtacttttctcatttcgtcccagtttttctccccgccaaacccaccactacccctgctcctaggcctcccactcccgcacccaccattgcttcccaacctctgtcattgaggcccgaagcctgcagacctgccgcaggaggggctgtgcacacccgcggtctggattttgcttgtgatatctacatttgggcccctttggccggaacctgcggagtgttgttgctgagccttgttatcacgttgtactgtaatcacagaaacagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 78 - protein, 518 aa, Homo sapiens
MALPVTALLLPLALLLHAARPQVQLVQSGAEVKKPGASVKVSCKASGYTFTGYYIHWVRQAPEQGLEWMGWINPNSGGTNYAQKFQGRVTMARDTSISTVYMDLSRLRSDDTAVYYCARIRGGNSVFDYWGQGTLVTVSSGGGGSGGGGSGGGGSDIVMTQSPDSLAVSLGERATINCKSTQSILYTSNNKNFLAWYQQKPGQPPKLLISWASIRESGVPDRFSGSGSGTDFALTISSLQAEDVAVYYCQQYFSTMFSFGQGTKLEIKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - DNA, 1461 bp, Homo sapiens
atggcactccccgtaactgctctgctgctgccgttggcattgctcctgcacgccgcacgcccgcaggtgcagctccaagagtcaggaccaggacttgtcaaaccaagccagaccctcagccttacctgcaccgtcagcgggggctccatcagctctggggcttactactggacatggatacgacagcatcccggtaaaggtctggagtggatcgggtacatacactatagtggttccacatattctaatccatctcttaagagtcgaattacaatttcactcgatacttcaaagaatcagttcagcttgaaactgaactccgtgaccgcggctgacaccgccgtgtactactgtgcacgccaagaggattatggcggactgttcgattattgggggcagggaactctcgtgacagtgagctccggcgggggcggcagcggtgggggtggaagtggtggagggggcagcgagatcgtgatgacccagagtcctgccacactgtcagtgagtcctggggagcgaatcacactttcctgtcgagcgtctcagtccgtgaccacggacctggcgtggtaccagcagatgccaggccaggcgccaagactcctgatctacgacgcttctacccgcgctactggtttccccgccagattctccggaagcgggtccgggacggattttacacttaccatctcttcattgcaggctgaggattttgccgtgtactactgtcagcattacaaaacctggcccctcactttcgggggcggaacaaaagtggaaattaaacgggcagcagctattgaggtgatgtacccacccccctacctggacaacgagaaatccaatggcaccatcatccacgttaagggtaagcacttgtgtccctcaccactcttccctgggcctagcaagccattctgggtcctggtggtcgtgggaggcgtgctggcctgctattccctcctggttaccgttgcctttatcatattttgggtcagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 80 - protein, 486 aa, Homo sapiens
MALPVTALLLPLALLLHAARPQVQLQESGPGLVKPSQTLSLTCTVSGGSISSGAYYWTWIRQHPGKGLEWIGYIHYSGSTYSNPSLKSRITISLDTSKNQFSLKLNSVTAADTAVYYCARQEDYGGLFDYWGQGTLVTVSSGGGGSGGGGSGGGGSEIVMTQSPATLSVSPGERITLSCRASQSVTTDLAWYQQMPGQAPRLLIYDASTRATGFPARFSGSGSGTDFTLTISSLQAEDFAVYYCQHYKTWPLTFGGGTKVEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - DNA, 1434 bp, Homo sapiens
atggcactccccgtaactgctctgctgctgccgttggcattgctcctgcacgccgcacgcccgcaggtgcagttgcaggagagcgggccaggcctggtgaagcccagccaaacactgagcctcacctgtactgtgtccggtggtagcatttccagcggggcgtattattggacatggatacgccaacaccctggaaaagggttggagtggattggatacatccattattctgggtccacctatagtaacccttctctcaagtctcgcattactattagtttggatacctctaagaatcagtttagtctgaagctgaacagtgtaaccgccgccgacaccgcggtctactactgtgctaggcaggaggattacgggggactgttcgattactggggccaggggacattggtcaccgtttcaagcgggggcggcggatctggcggagggggatctggaggcggaggctctgagatcgtaatgactcagagcccagccaccctgtccgtctctcccggcgaacgcatcactctgagctgtagggcatcacagtctgttaccacagatctggcttggtatcaacaaatgcctgggcaggccccgcgactgttgatttatgacgcctctacgcgggccacaggatttcctgcccggttctccgggtctggttctggcaccgattttaccttgacaatcagtagcttgcaggcagaagatttcgctgtgtattactgccaacattataagacatggcctttgacattcggcgggggaaccaaagtggagatcaaacgcgccgcagccctggacaatgagaagtctaatgggaccatcattcacgtcaaagggaaacacctgtgcccctctcctctgttcccaggcccttctaagcccttctgggttctcgtggtggtgggcggtgtcctggcctgctattcccttcttgtgacagtggcctttatcattttttgggtgagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 82 - protein, 477 aa, Homo sapiens
MALPVTALLLPLALLLHAARPQVQLQESGPGLVKPSQTLSLTCTVSGGSISSGAYYWTWIRQHPGKGLEWIGYIHYSGSTYSNPSLKSRITISLDTSKNQFSLKLNSVTAADTAVYYCARQEDYGGLFDYWGQGTLVTVSSGGGGSGGGGSGGGGSEIVMTQSPATLSVSPGERITLSCRASQSVTTDLAWYQQMPGQAPRLLIYDASTRATGFPARFSGSGSGTDFTLTISSLQAEDFAVYYCQHYKTWPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - DNA, 1542 bp, Homo sapiens
atggcactccccgtaactgctctgctgctgccgttggcattgctcctgcacgccgcacgcccgcaggtacagttgcaggaaagcggccccggccttgtaaaaccaagccagactctcagtttgacttgcaccgtctcaggaggaagcatttccagtggggcttattattggacttggattcggcagcatcctgggaaagggttggaatggatcggttatattcattatagcggtagcacctattccaatccgtctttgaaaagcagaatcactatttcactcgacacctctaagaaccagttcagtctcaaactgaactccgtgacagcggccgacacagctgtgtactactgtgcacggcaagaagattatggggggctgttcgattattggggccaaggcacactggtgacagtatcaagcggtggaggaggctccgggggcggaggaagtggaggcggggggagcgaaattgtgatgacccagtctccagccacgctgtcagtgtctccgggagaacgcataaccctctcctgccgggccagtcagtccgtcacgaccgatttggcttggtatcaacagatgcctgggcaggccccccgcttgctgatctatgacgcctccaccagagcaactggtttccccgcccggttcagcggatctggaagcggtacagattttacacttaccatctcatcattgcaagctgaggattttgccgtgtactactgccagcactacaagacctggcctttgacgttcggcggcggaacaaaagtggagattaaaagagccgctgccctcagtaactcaatcatgtactttagtcactttgtgcctgtgtttctgccagcaaagccaacaaccacaccagcaccccgccctccaacgcctgccccaaccatcgcctcccagcctctgagcttgaggcctgaggcttgtcgcccagctgctggaggtgctgtgcatacacgaggactggatttcgcctgcgatatctatatctgggcaccacttgccggtacttgtggtgtgttgctgctctcactggtcatcacgctgtactgtaaccataggaatagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgctag

SEQ ID NO: 84 - protein, 513 aa, Homo sapiens
MALPVTALLLPLALLLHAARPQVQLQESGPGLVKPSQTLSLTCTVSGGSISSGAYYWTWIRQHPGKGLEWIGYIHYSGSTYSNPSLKSRITISLDTSKNQFSLKLNSVTAADTAVYYCARQEDYGGLFDYWGQGTLVTVSSGGGGSGGGGSGGGGSEIVMTQSPATLSVSPGERITLSCRASQSVTTDLAWYQQMPGQAPRLLIYDASTRATGFPARFSGSGSGTDFTLTISSLQAEDFAVYYCQHYKTWPLTFGGGTKVEIKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 993 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 86 - DNA, 63 bp, Homo sapiens
atggcactccccgtaactgctctgctgctgccgttggcattgctcctgcacgccgcacgcccg

SEQ ID NO: 87 - protein, 21 aa, Homo sapiens
MALPVTALLLPLALLLHAARP

SEQ ID NO: 88 - DNA, 45 bp, Homo sapiens
ggcggtggaggctccggaggggggggctctggcggagggggctcc

SEQ ID NO: 89 - protein, 15 aa, Homo sapiens
GGGGSGGGGSGGGGS

SEQ ID NO: 90 - DNA, 54 bp, Homo sapiens
gggtctacatccggctccgggaagcccggaagtggcgaaggtagtacaaagggg

SEQ ID NO: 91 - protein, 18 aa, Homo sapiens
GSTSGSGKPGSGEGSTKG

SEQ ID NO: 92 - protein, 126 aa, Homo sapiens
AAGCGCGGCAGGAAGAAGCTCCTCTACATTTTTAAGCAGCCTTTTATGAGGCCCGTACAGACAACACAGGAGGAAGATGGCTGTAGCTGCAGATTTCCCGAGGAGGAGGAAGGTGGGTGCGAGCTG

SEQ ID NO: 93 - protein, 42 aa, Homo sapiens
KRGRKKLLYIFKQPFMRPVQTTQEEDGCSCRFPEEEEGGCEL

SEQ ID NO: 94 - protein, 37 aa, Homo sapiens
RRDQRLPPDAHKPPGGGSFRTPIQEEQADAHSTLAKI

SEQ ID NO: 95 - DNA, 6762 bp, Artificial Sequence
  Synthetic
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccggggatggcgcgccagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagccgccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgcgatcgcggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtccttcgaagtagatctttgtcgatcctaccatccactcgacacacccgccagcggccgctgccaagcttccgagctctcgaattaattcacggtacccaccatggcctagggagactagtcgaatcgatatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtccttttcatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggttaattaaagtacctttaagaccaatgacttacaaggcagctgtagatcttagccactttttaaaagaaaaggggggactggaagggcgaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcaggcatgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttggcgcgccatcgtcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 96 - protein, 15 aa, Homo sapiens
IPYYGSGSHNYGMDV

SEQ ID NO: 97 - protein, 5 aa, Homo sapiens
NYGMH

SEQ ID NO: 98 - DNA, 357 bp, Homo sapiens
caggtgcagctggtgcagtctggggctgaggtgaagaagcctggggcctcagtgaaggtctcctgcaaggcttctggatacaccttcaccggctactatatacactgggtgcgacaggcccctgaacaagggcttgagtggatgggatggatcaaccctaacagtggtggcacaaactatgcacagaagtttcagggcagggtcaccatggccagggacacgtccatcagcacagtttacatggacctgagcaggctgagatctgacgacacggccgtgtattactgtgcgagaatacgcggtggtaactcggtctttgactactggggccagggaaccctggtcaccgtctcctca